(19) United States
(12) Patent Application Publication
(10) Pub. No.: US 2026/0124223 A1
(43) Pub. Date: May 7, 2026

(54) CANCER IMMUNOTHERAPIES

(71) Applicant: Northwestern University, Evanston, IL (US)

(72) Inventors: Victor Andres Arrieta GONZALEZ, Evanston, IL (US); Roger STUPP, Evanston, IL (US); Christina Marie AMIDEI, Evanston, IL (US); Adam Mendel SONABEND WORTHALTER, Evanston, IL (US); Kwang-Soo KIM, Evanston, IL (US)

(73) Assignee: Northwestern University, Evanston, IL (US)

(21) Appl. No.: 19/117,789

(22) PCT Filed: Oct. 2, 2023
(86) PCT No.: PCT/US2023/034299

§ 371 Date: Apr. 2, 2025

Related U.S. Application Data
(60) Provisional application No. 63/412,718, filed on Oct. 3, 2022.
(60) Provisional application No. 63/539,291, filed on Sep. 19, 2023.

(51) Int. Cl. A61K 31/704; A61K 39/00; A61K 39/395; A61K 41/00; A61P 35/00

(52) U.S. Cl. CPC A61K 31/704; A61K 39/3955; A61K 41/0047; A61P 35/00; A61K 2039/505

(57) ABSTRACT

The present disclosure generally relates to technologies for treating cancer, including brain cancers such as a glioblastoma, methods of increasing the concentration of anthracy clines and immune checkpoint modulators in the brain of a subject, and methods of improving use of immune checkpoint modulators in brain cancers. Also disclosed herein are compositions and methods for treating a brain tumor.

### CROSS REFERENCE TO RELATED APPLICATIONS

This application claims the benefit of and priority to U.S. Provisional Patent Application No. 63/412,718, filed on Oct. 3, 2022, and U.S. Provisional Patent Application No. 63/539,291, filed on Sep. 19, 2023, the contents of each of which are incorporated herein by reference in its entirety.

### GOVERNMENT LICENSING RIGHTS STATEMENT

This invention was made with Government support under Grant Nos. CA264338 and CA245969 awarded by the National Institutes of Health (NIH). The government has certain rights in the invention.

### BACKGROUND

Many cancers, including glioblastoma, remain resistant to immunotherapy. Recent clinical trials demonstrate unprecedented efficacy against immunologically inactive (‘cold’) tumors using botensilimab, an Fc-enhanced anti-CTLA-4 (FcE-aCTLA-4) antibody with a novel mechanism of action. Immune checkpoint blockade (ICB) targeting cytotoxic T-lymphocyte antigen 4 (aCTLA-4) is an established form of immunotherapy and has been investigated in clinical trials for GBM (NCT02311920, NCT04396860) (21-23). Further, recent studies have demonstrated that the ability of aCTLA-4 antibody to co-engage FcγRIIIA on antigen-presenting cells (APCs) such as dendritic cells or macrophages is critical for promoting T cell priming and activation (24) and to drive myeloid activation and type I interferon signaling (30), key mechanisms that are thought to be important for treating poorly immunogenic and ‘cold’ tumors. The importance of co-engaging FcγRIIIA with aCTLA-4 have also been shown clinically (29). Moreover, conventional aCTLA-4 antibodies may have limited therapeutic activity due to suboptimal co-engagement of activating FcγRs.

There is a need for improved immunotherapy methods for treating cancer, including brain tumors, and in particular, gliomas and GBM.

### SUMMARY OF THE INVENTION

The present disclosure provides, among other things, methods for treating cancer, including but not restricted to primary brain cancers such as a glioblastoma or glioma, methods of increasing the concentration of anthracyclines and immune checkpoint modulators in the brain of a subject, and methods of improving use of immune checkpoint modulators in cancer. Also disclosed herein are compositions and methods for treating a cancer.

In one embodiment, the present disclosure provides a method for treating cancer in a subject in need thereof comprising, consisting of, or consisting essentially of administering to the subject a therapeutically effective amount of an anthracycline. In some embodiments, the method further comprises, consists of, or consists essentially of administering to the subject a therapeutically effective amount of a checkpoint inhibitor. In some aspects, the checkpoint inhibitor comprises, consists of, or consists essentially of an anti-CTLA-4 antibody. In some embodiments, the method further comprises, consists of, or consists essentially of administering to the subject a therapeutically effective amount of an anti-PD-1 antibody. In some embodiments, the method comprises, consists of, or consists essentially of disrupting the blood-brain barrier of the subject by administering low-intensity pulsed ultrasound and microbubbles.

In some embodiments, the effective amount of the anthracycline is below the established human cytotoxic or cardiotoxic amount. In some embodiments, the therapeutically effective amount of the anthracycline comprises 30 mg or less. In some aspects, the effect amount of the anthracycline comprises 35 mg or less, or 40 mg or less, or 45 mg or less, or 50 mg or less. In some embodiments, the therapeutically effective amount of the anthracycline comprises an amount that results in a tumor concentration of at least 0.1 mM of anthracycline 0.2 mM or greater, 0.3 mM or greater, or 0.4 mM or greater, or 0.5 mM or greater. In some embodiments, the therapeutically effective amount of the anthracycline comprises an amount that results in a blood serum concentration of at least 0.1 mM of anthracycline, 0.2 mM or greater, 0.3 mM or greater, or 0.4 mM or greater, or 0.5 mM or greater. In some aspects the effective amount of the anthracycline does not result in cytotoxicity in the subject. In some aspects, the effective amount of the anthracycline is a non-cytotoxic dose of an anthracycline. In some aspects, the effective amount of the anthracycline is sufficient to increase expression of FcyRIIA in the subject.

In some embodiments, the therapeutically effective amount of the anthracycline and the therapeutically effective amount of the checkpoint inhibitor are administered simultaneously. In other embodiments, the therapeutically effective amount of anthracycline is administered immediately after the therapeutically effective amount of the checkpoint inhibitor is administered. In some embodiments, the therapeutically effective amount of anthracycline is administered before the therapeutically effective amount of the checkpoint inhibitor is administered. In some embodiments, there is a period of time between administering the therapeutically effective amount of anthracycline and therapeutically effective amount of the checkpoint inhibitor, wherein the period of time comprises, consists of, or consists essentially of about 1 to about 8 hours.

In some embodiments, the therapeutically effective amount of the anthracycline, the therapeutically effective amount of the checkpoint inhibitor, the therapeutically effective amount of the anti-PD-1 antibody, and the low-intensity pulsed ultrasound and microbubbles are administered simultaneously.

In some embodiments, the therapeutically effective amount of the anthracycline and the low-intensity pulsed ultrasound and microbubbles are administered simultaneously.

In some embodiments, the therapeutically effective amount of anthracycline is administered immediately after the low-intensity pulsed ultrasound and microbubbles are administered.

In some embodiments, the therapeutically effective amount of the checkpoint inhibitor antibody or the anti-PD-1 antibody is administered first, then the low-intensity pulsed ultrasound and microbubbles are administered, and the therapeutically effective amount of the anthracycline is administered last.

In some embodiments, there is a period of time between administering the therapeutically effective amount of the anti-PD-1 antibody and administering the low-intensity pulsed ultrasound and microbubbles. In some embodiments, the period of time is about 1 hour to about 8 hours.

In some embodiments, the anthracycline comprises, consists of, or consists essentially of doxorubicin. In some embodiments, the doxorubicin comprises, consists of, or consists essentially of liposomal doxorubicin.

In some embodiments, disrupting the blood-brain barrier increases the concentration of the anthracycline in the brain of the subject relative to the concentration of the anthracycline in the brain of the subject in the absence of disrupting the blood-brain barrier.

In some embodiments, disrupting the blood-brain barrier increases the concentration of the anti-PD-1 antibody in the brain of the subject relative to the concentration of the anti-PD-1 antibody in the brain of the subject in the absence of disrupting the blood-brain barrier.

In some embodiments, the microbubbles are administered intravenously.

In some aspects, the anti-CTLA-4 or anti-PD1 antibody comprises, consists of, or consists essentially of a humanized antibody. In some aspects, the antibody comprises, consists of, or consists essentially of a recombinant or engineered antibody. In some aspects, the anti-CTLA-4 antibody comprises, consists of, or consists essentially of ipilimumab.

In some embodiments, the low-intensity pulsed ultrasound is administered by an ultrasound device. In some embodiments, the ultrasound device is implanted in a cranial window in the skull of the subject.

In some embodiments, the subject was previously treated with a radiotherapy, a chemotherapy, an immunotherapy, or any combination thereof.

In some embodiments, the cancer comprises, consists of, or consists essentially of a glioma. In some aspects, the glioma comprises, consists of, or consists essentially of a glioblastoma. In some embodiments, the glioblastoma comprises, consists of, or consists essentially of a recurrent glioblastoma.

In some embodiments, the low-intensity pulsed ultrasound is administered in a plurality of pulsed steps.

In some embodiments, the method further comprises monitoring the subject for one or more toxicities.

In some embodiments, the method further comprises assessing disruption of the blood-brain barrier by contrast magnetic resonance imaging.

In one aspect, the present disclosure provides a method of upregulating the expression of FcγRIIIA in a subject comprising, consisting of, or consisting essentially of administering a therapeutically effective amount of an anthracycline, and administering a therapeutically effective amount of a checkpoint inhibitor to the subject wherein the effective amount of the anthracycline is a sub-therapeutic amount.

### BRIEF DESCRIPTION OF THE DRAWINGS

FIG. 1A-1K demonstrates that DOX induces the upregulation of antigen-presenting molecules in GBM human cells. FIG. 1A shows a clinical course of recurrent GBM patients that underwent surgery for tumor resection (pre-treatment GBM sample) and skull implantation of the SonoCloud-9 US device for adjuvant treatment with a previous chemotherapy. Upon detection of tumoral progression, induction treatment with liposomal DOX delivered with US/MB was initiated to treat the recurrent tumor followed by additional treatment cycles with both liposomal DOX and pembrolizumab delivered by US/MB. The tumor exposed to these therapies was resected (on-treatment GBM sample) and further analyzed. FIG. 1B shows (left) schematic and MRI of the placement site of the SonoCloud-9 US device in a GBM patient. (right) 3D MRI reconstruction representing the method to map the sampling sites of peritumoral brain tissue that were subjected to sonication (green circle) and those that were not sonicated (blue circle) obtained during neurosurgery. FIG. 1C shows a bar plot representing the concentration of DOX in plasma and tumors obtained during surgery before DOX treatment and 48 hours after DOX infusion. DOX concentrations in sonicated and non-sonicated peritumoral brain regions are also shown in the bar plot for reference. FIG. 1D shows a bar plot representing the fold change in DOX concentration in non-sonicated (n=4 brain samples) and sonicated peritumoral brain tissues (n=8 brain samples) from 2 GBM patients. A mixed effects model was constructed considering sonication as a fixed effect and patients as a random effect influencing fold change of DOX concentration. P value was obtained by likelihood ratio tests of the full model with the effect in question against the model without the effect in question. FIG. 1E shows a schematic of the flow cytometry experiment performed to assess the effect of different DOX concentrations on the expression of antigen-presenting molecules in the PDX cell lines, GBM6 and GBM63. FIG. 1F-1G (left) shows bar plots representing the expression of HLA ABC and HLA-DR assessed as MFI values in GBM6 (FIG. 1F) and GBM63 (FIG. 1G). n=3 biological replicates per condition. (right) Representative histograms showing the expression of HLA ABC and HLA-DR in the control and 0.15-0.6 μM DOX-treated cells. Histograms are representative data from three biological replicates. P values obtained by one-way ANOVA with post hoc Dunnett's multiple comparisons test. FIG. 1H shows Dot plot showing the cell density of SOX2⁺ HLA-ABC⁺ cells in pre-treatment GBM samples and on-treatment GBM samples. n=3 paired GBM samples. P values obtained by paired one-tailed t test. FIG. 1I shows dot plot showing the cell density of SOX2⁺ HLA-DR⁺ cells in pre-treatment GBM samples and on-treatment GBM samples. n=3 paired GBM samples. P values obtained by paired one-tailed t test. FIG. 1J shows UMAP plots showing the expression of SOX2, HLA ABC, and HLA DR obtained from multiplex immunofluorescence data in pre-treatment and on-treatment GBM samples. The color key indicates expression levels. Each dot represents an individual cell. n=102,850 cells from 3 pre-treatment GBM samples and n=121,576 cells from 3 on-treatment GBM samples. FIG. 1K shows representative multiplex immunofluorescence images illustrating tumor regions containing SOX2⁺, HLA ABC⁺, and HLA DR⁺ cells in pre-treatment and on-treatment GBM samples. Data are presented as mean±SEM. (FIG. 1C, 1D, 1F, 1G)

FIG. 2A-2N demonstrates that US/MB-mediated delivery of liposomal DOX induces an IFN-γ phenotype in glioma-infiltrating CD11b⁺ myeloid cells. FIG. 2A (left) Experimental outline and therapeutic scheme employed to treat GL261-bearing mice with liposomal DOX and with and without US/MB. (right) Flow cytometry plots showing the immune cell populations that were analyzed in murine gliomas. FIG. 2B-2C show bar plots showing the percentage of microglia (FIG. 2B) and macrophages (FIG. 2C) producing IFNγ⁺ from groups treated with different doses of liposomal DOX (1, 2, and 5 mg/kg) with or without US/MB. FIG. 2D shows representative scatter plots from each treatment group showing the gating strategy to determine IFNγ myeloid cells. FIG. 2E provides tSNE plots showing a representation of total cells along with the expression of cellular markers (CD45, CD4, CD8, and CD11b) and IFNγ. The color key indicates expression levels. FIG. 2F-2G show bar plots showing the percentage of MHC I⁺ (FIG. 2F) and PD-L1⁺ (FIG. 2G) cells from microglia (left) and MDMs (right) from mouse groups treated with different doses of liposomal DOX.

FIG. 211-2J show bar plots representing the expression of IFNγ (FIG. 211), HLA ABC (FIG. 2I), and PD-L1 (FIG. 2J) assessed as MFI values in HMC3 cells. n=3 biological replicates per condition. FIG. 2K shows dot plots showing the cell density of the TMEM119⁺ IFN-γ+ in pre-treatment and on-treatment GBM samples. n=4 paired tumors. One dot represents one tumor. P values by paired one-tailed t test. FIG. 2L shows PCA plots displaying myeloid cells (CD163 and TMEM119 cells) from multiplex immunofluorescence data from both pre-treatment and on-treatment GBM samples. FIG. 2M shows PCA plots displaying the production of IFN-γ by TMEM119 and CD163 cells in pre-treatment and on-treatment GBM samples. n=116,717 myeloid cells in pre-treatment GBM samples, n=95,719 myeloid cells in on-treatment GBM samples. The color key indicates the expression levels. FIG. 2N shows representative multiplex immunofluorescence images illustrating tumor regions containing SOX2⁺, TMEM119⁺, and IFN-γ⁺ cells in pre-treatment and on-treatment GBM samples. Each dot represents one mouse in FIGS. 2B, 2C, 2F, and 2G. P values in FIGS. 2B, 2C, 2F, 2G, and 2I were derived from one-way ANOVA with post hoc Tukey's multiple comparison test. Data were presented as mean±SEM (FIG. 2B, 2C, 2F, 2G, 2I).

FIG. 3A-3G demonstrate US/MB-based blood-brain barrier (BBB) opening enhances the efficacy of liposomal DOX in murine glioma models. FIG. 3A show a therapeutic scheme employed to treat GL261-bearing mice with liposomal DOX with US/MB. FIG. 3B shows Kaplan-Meier curves showing survival of glioma-bearing mice treated with liposomal DOX with and without US/MB as well as the tumor rechallenge experiment performed in long-term survivors. P values by log-rank test. FIG. 3C-3D shows percentage of CD4⁺ (FIG. 3C) and CD8⁺ (FIG. 3D) T cells in long-term survivors and control groups. FIG. 3E-3F show percentage of CD4⁺ (FIG. 3E) and CD8⁺ (FIG. 3F) T cells expressing IFN-γ and TNF-α in the brain of long-term survivor mice after tumor re-challenge in the contralateral brain hemisphere. FIG. 3G shows representative flow cytometry plots of CD4⁺ and CD8⁺ IFN-γ⁺TNF-α⁺ cells in different treatment groups. Data are presented as mean±SD. (3C, 3D, 3E, 3F). P values by one way-ANOVA with post hoc Tukey's multiple comparisons test in FIG. 3C, 3D, 3E, 3F.

FIG. 4A-4I demonstrates US/MB increases the concentration of PD-1 antibody in the brain. FIG. 4A shows a scheme showing the experimental procedure to evaluate the concentration of nivolumab by ELISA in BBB-intact brains of mice injected with fluorescein subjected and not subjected to sonication. FIG. 4B shows macroscopic fluorescent images of mouse brains from 4 groups that receive fluorescein, nivolumab, without and with US/MB obtained after 1 and 4 hours. FIG. 4C-4D show bar plots showing the concentration of nivolumab in plasma (FIG. 4C) and in the brain (FIG. 4D) in the following groups: US/MB alone, nivolumab without US/MB, nivolumab with US/MB after 1 hour, and nivolumab with US/MB after 4 hours. FIG. 4E (top) shows a therapeutic scheme and (bottom) Kaplan-Meier curve of CT2A-bearing mice treated with aPD-1 delivered with and without US/MB. Median survival of ISO Ab: 27 days, median survival of aPD-1: 29 days, medium survival of US/MB+aPD-1: 49 days. n=10 mice in control group (ISO Ab), n=9 mice in the aPD-1 group, n=9 mice in the US/MB+aPD-1 group. P values by Log-rank test. FIG. 4F shows bar plot showing the pembrolizumab levels in plasma before, 48 hours and 30 min after immunotherapy administration. FIG. 4G (left) shows a bar plot showing the concentration of pembrolizumab in the GBM samples acquired in a previous timepoint of tumoral progression and in subsequent recurrent GBM samples resected after 48 hours of immunotherapy administration. n=2 pretreatment tumor samples (black) and 7 tumor samples (green) from 2 GBM patients; (right) Dot plot representing the concentration of pembrolizumab in sonicated and non-sonicated peritumoral brain regions obtained after 48 hours of immunotherapy administration. n=2 non-sonicated and 3 sonicated peritumoral brain samples from 2 GBM patients. FIG. 4H shows a bar plot representing the fold change in pembrolizumab concentration in sonicated and non-sonicated peritumoral regions of GBM patients. MRIs of the peritumoral brain samples obtained during surgery employing neuronavigation covered and not covered by the sonication field represented as yellow squares. A mixed effects model was constructed considering sonication as a fixed effect and patients as a random effect influencing fold change of pembrolizumab concentration. P value was obtained by likelihood ratio tests of the full model with the effect in question against the model without the effect in question. n=3 sonicated and 2 non-sonicated peritumoral brain samples from 2 GBM patients. FIG. 4I shows an illustration of intracranial clinical course of a melanoma patient with two geographically separated brain tumor masses. The sonicated metastasis decreased in size after two sonication session, while the non-sonicated tumor mass continued growing until the patient received GammaKnife therapy. P values in 4C and 4D were derived from one way-ANOVA with post hoc Tukey's multiple comparisons test. Data are presented as mean±SD in FIGS. 4C, 4D, and 4G.

FIG. 5A-5D demonstrate enhanced delivery of liposomal DOX and aPD-1 by US/MB increase survival in glioma-bearing mice. FIG. 5A shows a therapeutic scheme employed to treat glioma-bearing mice with the combination of liposomal DOX and aPD-1 delivered with or without US/MB using GL261 and CT2A cells. FIG. 5B-5C show Kaplan-Meier curves showing survival of GL261-bearing mice treated with aPD-1, liposomal DOX with and without US/MB and the tumor rechallenge experiment performed in long-term survivors.

FIG. 5D shows a Kaplan-Meier curve showing survival of CT2A-bearing mice treated with the combinatorial therapy with aPD-1, the combinatorial therapy with and without US/MB. P values in 5B, 5C, and 5D were derived from log-rank test.

FIG. 6A-6B demonstrates US/MB-mediated delivery of liposomal DOX influences and requires tumor-infiltrating T cells. FIG. 6A Top: Bar plots showing the percentage of CD8⁺ and CD4⁺ T cells expressing IFNγ⁺ from non-treated and DOX-treated GBMs. Bottom: Scatter plots showing the expression of IFNγ⁺ by CD8⁺ and CD4⁺ T cells from non-treated and DOX-treated GBMs. The color key indicates normalized expression values of the indicated markers. (n=4 GBM samples treated with liposomal DOX+pembrolizumab+US/MB, n=7 GBM samples that did not receive treatment). P values derived from unpaired one-sided t test. FIG. 6B shows a therapeutic scheme and Kaplan-Meier curve representing the survival experiment and outcomes of CT2A-bearing mice in the context of Cd8^{+/+} and Cd8^{-/-} backgrounds treated with liposomal DOX and aPD-1 with US/MB. P value derived from Log rank test.

FIG. 7A-7D demonstrates methods to evaluate MHC I expression in human and murine glioma cell lines. FIG. 7A shows a gating strategy to evaluate expression of MHC I by GBM6 and GBM63. FIG. 7B shows bar plot showing the MFI values derived from DOX fluorescence in different concentrations of the anthracycline, IFNγ, temozolomide, and control groups in GBM6 and GBM63. FIG. 7C shows bar plots representing the expression of H2-Kb assessed as MFI values in GL261 (n=3 biological replicates per group) and CT2A (n=2 biological replicates per group). P values by one-way ANOVA with post hoc Dunnett's multiple comparisons test. FIG. 7D shows microscopy images of H&E staining and multiplex immunofluorescence staining representing tumor regions delineated by the neuropathologist that were used to quantify tumor and immune cell populations.

FIG. 8A-8C demonstrates the effect of US/MB in toxicity and survival of murine models. FIG. 8A Top: Macroscopic fluorescent images of non-tumor mouse brains of animals injected with fluorescein and subjected to sonication using two sonication pressures (0.2 and 0.3 mPa) and different MB doses (100, 150, and 200 μL). Bottom: Representative examples of mouse brains from the indicated sonication groups employing different parameters. Red arrows point to hemorrhages areas. FIG. 8B-8C shows Kaplan-Meier curves of glioma-bearing mice undergoing and not undergoing sonication employing GL216 (FIG. 8B) and CT2A (FIG. 8C) cells. P values derived from Log-rank test. n=10 glioma-bearing mice for each group (US/MB and control groups).

FIG. 9A shows a gating strategy used to analyze GBM-infiltrating immune cells in mice treated with different doses of liposomal DOX with and without US/MB. Flow cytometry plots used to analyze the production of cytokines and the expression of surface markers in murine immune cells. Immune cells were gated based on SSC and FSC parameters followed by exclusion of doublets. Next, live cells were gated based on low values of APC-Cy7 fluorescence intensity. Subsequently, live cells were gated based on the expression of CD45 and CD11b. Then, lymphocytes were gated based on CD45⁺ and CD11b⁻ followed by gating employing CD8⁺ and CD4⁺ markers. IFN-γ⁺, GZMb⁺, TNF-α⁺ and IL-10⁻ were evaluated on CD8⁺ and CD4⁺ T cells. Macrophages were gated based on CD45⁺ and CD11b⁺. Microglia were gated based on CD45⁻ and CD11b⁻. Macrophages and microglia were interrogated for the production IFN-γ⁺, TNF-α⁺ and IL-10⁺ as well as the expression of H2-Kb⁺ and PD-L1⁺.

FIG. 10A-10J shows the effect of liposomal DOX in tumor-infiltrating and peripheral immune cells. FIG. 10A-10B show bar plots representing the MFI values of IFN-γ⁺ in microglia (FIG. 10A) and macrophages (FIG. 10B). FIG. 10C-10D shows bar plots showing the percentage of cells TNFα⁺ and IL-1β⁺ from groups treated with different doses of liposomal DOX (1, 2, and 5 mg/kg) with or without US/MB in microglia (FIG. 10C) and macrophages (FIG. 10D). FIG. 10E shows tSNE plots showing abundance of cells from glioma-bearing mice along with expression of cellular markers (CD45, CD4, CD8, and CD11b) and DOX. The color key indicates expression levels. FIG. 10F-10G shows bar plots representing the percentage of CD8⁺ (FIG. 10F) and those that express IFN-γ⁺, GZMb⁺, and IL-1β⁺ (FIG. 10G) from groups treated with different doses of liposomal DOX (1, 2, and 5 mg/kg) with or without US/MB. FIG. 10H-10I show bar plots representing the percentage of CD4⁺ (FIG. 10H) T cells, and those that express IFN-γ⁺, TNF-α⁺, and IL-1β⁺ D (FIG. 10I) from groups treated with different doses of liposomal DOX (1, 2, and 5 mg/kg) with or without US/MB. FIG. 10J shows a dot plot showing the cell density of TMEM119⁺ HLA ABC⁺ and CD163⁺ HLA ABC⁺ cells in pre-treatment GBM samples and on-treatment GBM samples. n=4 paired GBM samples. P values in FIGS. 10A, 10B, 10C, 10D, 10F, 10G, 10H and 10I were derived from one way-ANOVA with post hoc Tukey's multiple comparisons test.

FIG. 11 shows a bar plot showing the number of IFNγ spots per 100,000 PBMCs extracted from GL261-bearing mice treated with the indicated doses of liposomal DOX. PBMCs from these animals were stimulated with GL261 cell lysate. Representative wells from the control and DOX 5 mg/kg are shown to the right of the bar plots. P values derived from one way-ANOVA with post hoc Tukey's multiple comparisons test.

FIG. 12 shows a gating strategy used to analyze T cells from human GBMs treated and not treated with liposomal DOX plus PD-1 antibody delivered with US/MB. Flow cytometry plots used to analyze the production of cytokines and the expression of surface markers on GBM-infiltrating T cells. Lymphocytes were gated based on SSC and FSC parameters followed by exclusion of doublets. Next, live cells were gated based on the Zombie NIR Viability staining and subsequently, T cells were gated base on the expression of CD45 and CD3. Then, CD3 T cells were analyzed for expression of CD8 and CD4 markers. CD8 and CD4 T cells were evaluated for the expression of IFNγ⁺.

FIGS. 13A-13H: Fc-enhanced anti-mouse CTLA-4 antibody exhibits increased binding to Fcγ receptors. Assessment of mouse reactive anti-CTLA4 antibody binding to Chinese hamster ovarian (CHO) cells genetically engineered to express mouse (FIGS. 13A, B, C, D) (FIG. 13E) Fcγ receptor (FcγR) I, (FIG. 13F) FcγRIIB, (FIG. 13G) FcγRIII or (FIG. 13H) FcγRIV. Cells were incubated with increasing concentrations of Fc-enhanced mouse reactive anti-CTLA4 antibody (clone 9D9, mIgG2b.DLE), anti-CTLA4 mIgG2b or an mIgG1 isotype control antibody (negative control). Binding was analyzed by flow cytometry using a fluorochrome conjugated anti-mouse F(ab′)2 secondary antibody.

FIGS. 14A-14J: FcγRs and CTLA-4 expression in murine and human glioma microenvironment. (FIG. 14A) Uniform manifold approximation and projection (UMAP) dimensionality reduction plot indicating cell categories from single-cell RNA sequencing analysis of human recurrent glioblastoma. Violin plot of FCGR3A (FIG. 14B) and CTLA-4 (FIG. 14C) expression across the cell types in human recurrent glioblastoma. (FIG. 14D) Multiplex immunofluorescence image of a human recurrent glioblastoma patient showing FcγRIIIA (CD16) expression on myeloid cell compartment (left; scale bar=1 mm, right; scale bar=50 μm). (FIG. 14E) Quantification graph showing the percentages of FcγRIIIA+ cells out of infiltrating macrophage/microglial markers (CD11c, CD68). Ordinary one-way ANOVA test was used. (****, p<0001) (FIG. 14F) UMAP plot indicating cell categories of CT-2A mouse glioblastoma scRNAseq for further analysis. (FIG. 14G) UMAP plots showing expression of Fcgr4 (left), violin plot addressing comparison of Fcg4 expression in different cell types from mouse CT-2A tumor (right). (FIG. 14H) Differential expression analysis of Fcgrl, Fcgr2b, Fcgr3, Fcgr4, Ctla4, Pdcdl, and Cd274 from different cell populations. (FIG. 141) UMAP plots showing expression of Ctla4 (left), violin plot addressing comparison of Ctla4 expression in different cell types from mouse CT-2A tumor (right). (FIG. 14J) Comparison of Fcgrs expression by total RNA sequencing from mouse CT-2A tumor infiltrating Grl+ myeloid cells and splenic Grl+ myeloid cells. Each column represents an independent experiment where myeloid cells from 10 mice were pulled for analysis (data obtained from Miska et al., Science Advances 2021 DOI: 10.1126/sciadv.abc8929).

FIGS. 15A-15H: Fc-enhanced anti-CTLA-4 promotes phagocytosis of Tregs ex vivo and in vivo. (FIG. 15A) Schematic illustration of cell preparation. TAMs generated from bone marrow progenitor cell, differentiated with M-CSF and conditioned with CT-2A mouse glioblastoma cell cultured media. (FIG. 15B) Graph showing time-dependent cell overlap (phagocytosis) between Tregs (green) and macrophages (red) with isotype control, parental anti-CTLA-4, and FcE anti-CTLA-4 antibody. (FIG. 15C) Graph showing time-dependent cell overlap (phagocytosis) between CD4+ T cells (non-Tregs, green) and macrophages (red) with isotype control, parental anti-CTLA-4, and FcE anti-CTLA-4 antibody. Ordinary two-way ANOVA with multiple comparison was performed for graphs (FIGS. 15B and 15C). (FIG. 15D) Schematic illustration of immunophenotype analysis design. (FIG. 15E) Flow cytometry analysis showing Tregs (Foxp3+) in CD4+ T cells at day 21. (FIG. 15F) Tumor-specific Treg ratio was plotted at two different time points (left) and the Treg ratio in the spleen on day 14 and 21 (right). Each comparison was analyzed by unpaired Student's T-test. (FIG. 15G) Time-dependent PD-1 downregulation in CD4 T cells. At each time point, the mean fluorescent intensity of PD-1 was calculated and plotted (left), and the representative histogram (right). (FIG. 15H) Time-dependent PD-1 downregulation in CD8 T cells. At each time point, the mean fluorescent intensity of PD-1 was calculated and plotted (left), and the representative histogram (right). Unpaired T-test was used for statistical analysis. Data indicate mean±SD (FIGS. 15F-15H). Statistical significance is depicted as ns: not statistically significant, *p<0.05, **p<0.01, ***p<001, ****p<0.0001.

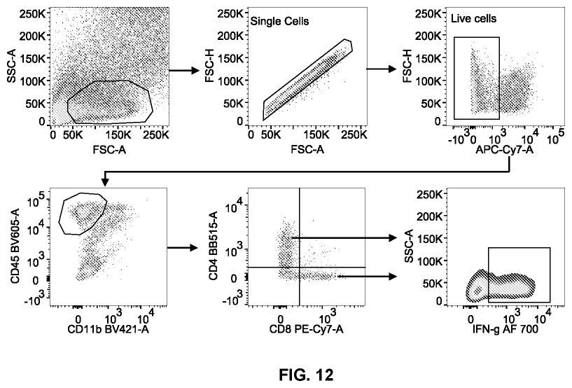

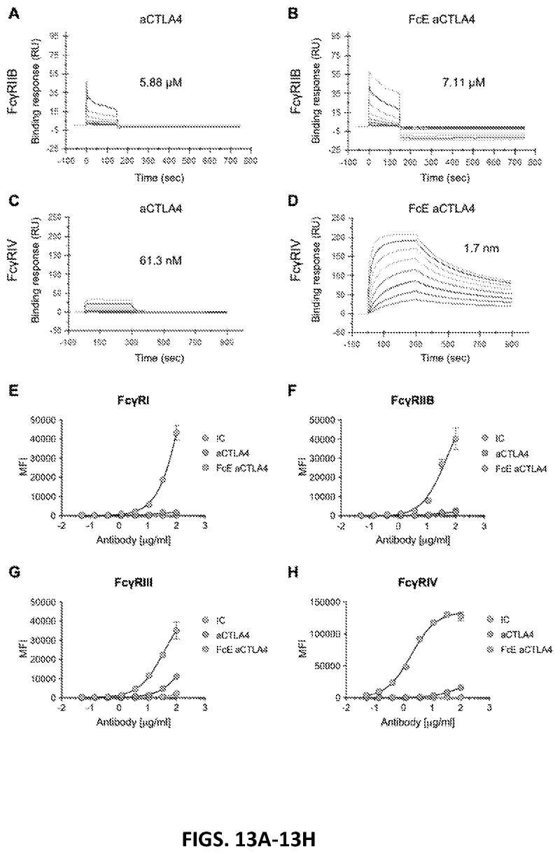

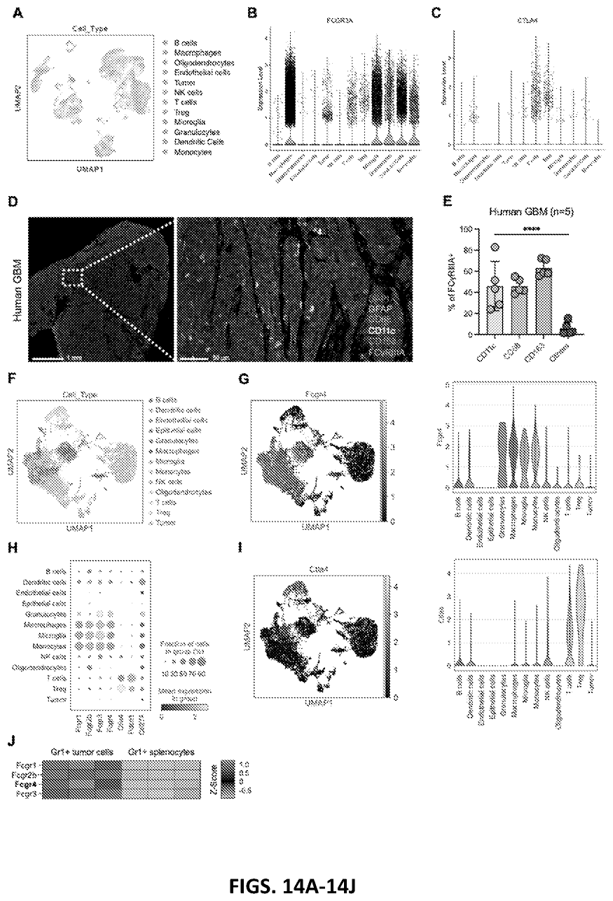

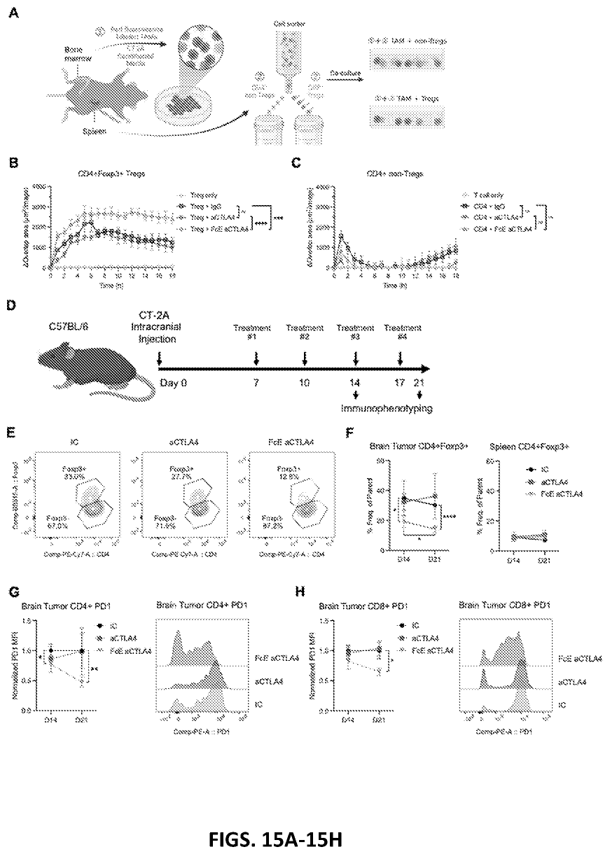

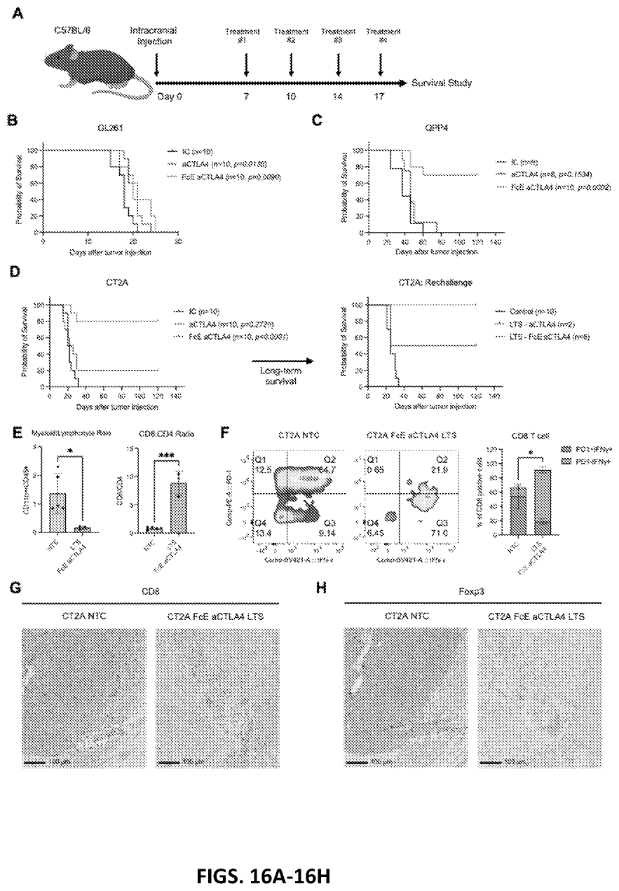

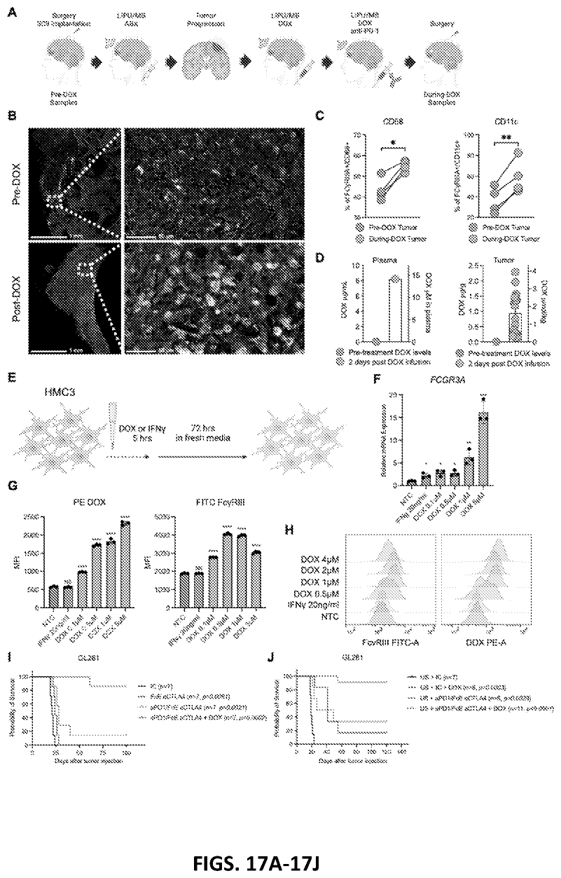

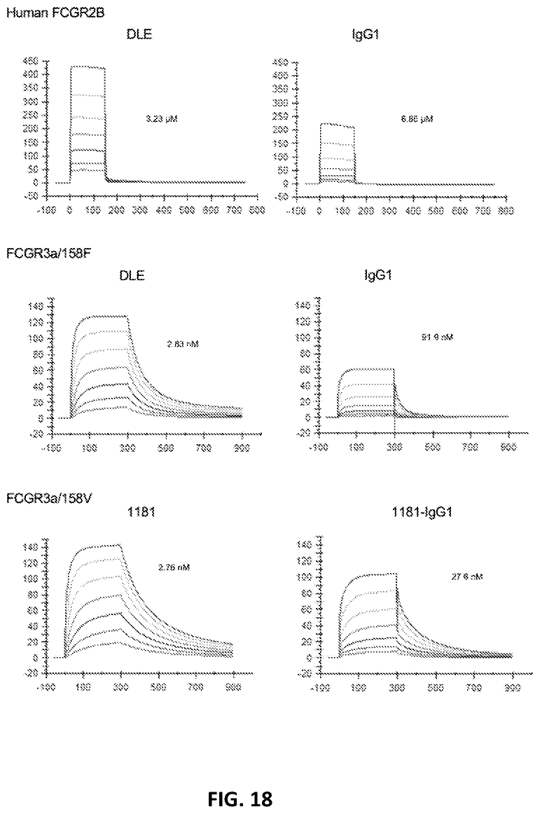

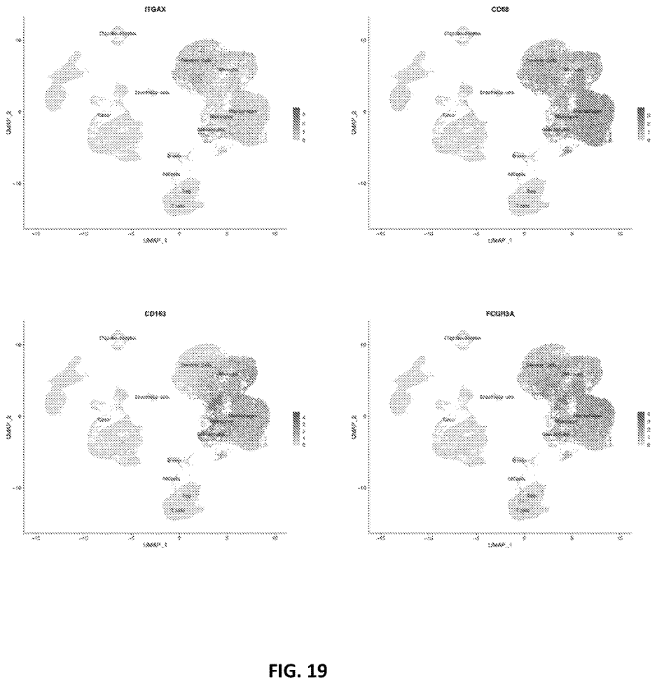

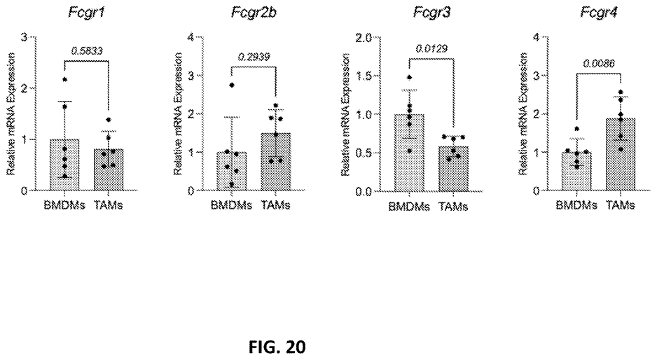

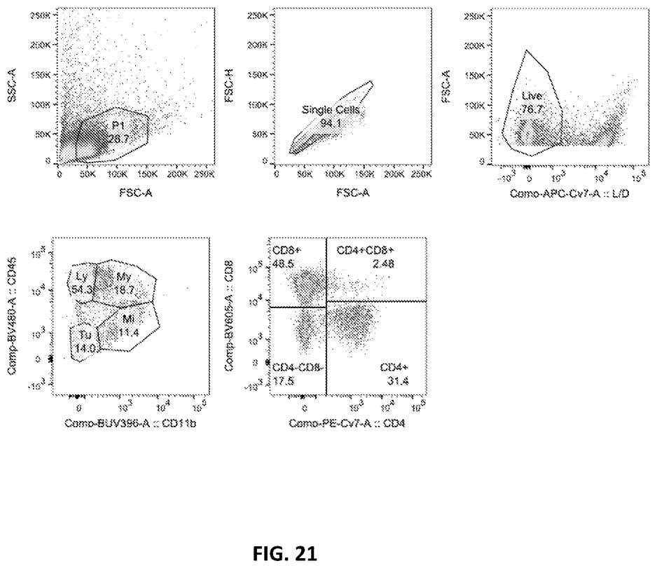

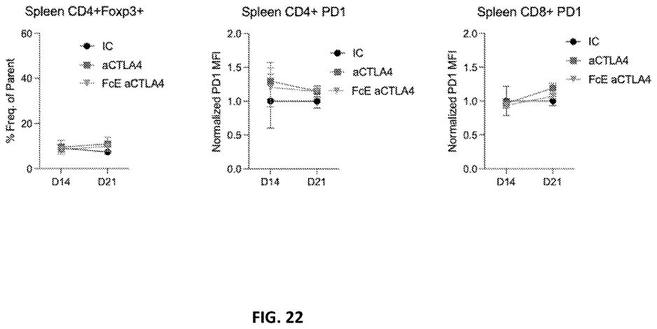

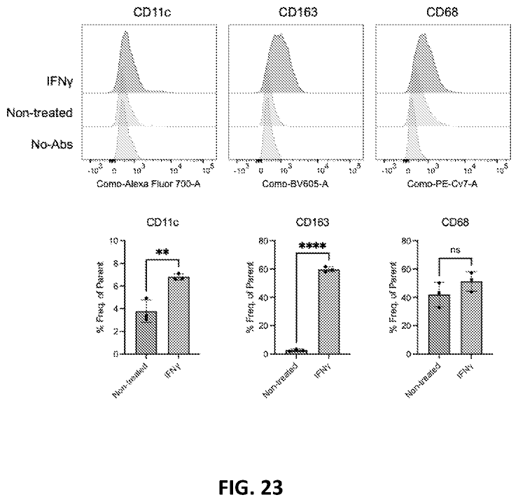

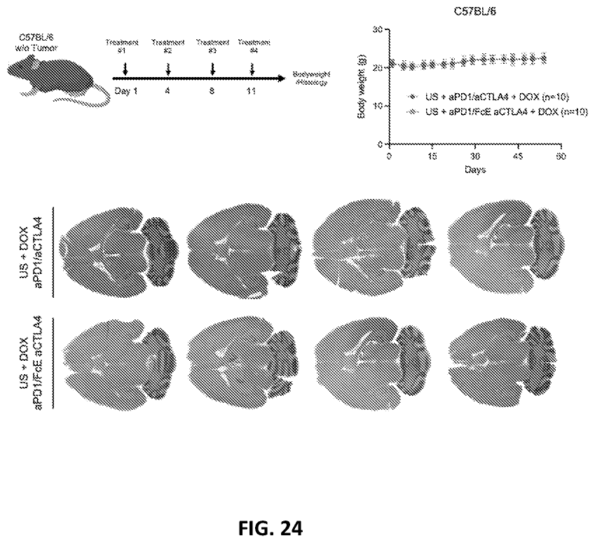

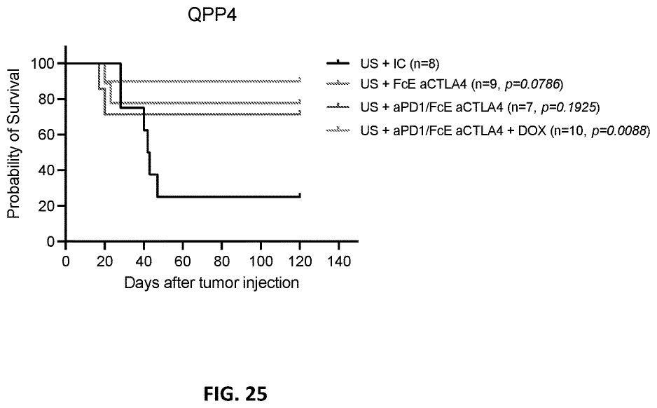

FIGS. 16A-16H: Fc-enhanced anti-CTLA-4 exhibits enhanced efficacy in immune-resistant murine glioma models. (FIG. 16A) Schematic illustration of treatment and survival study design. The efficacy test of the FcE anti-CTLA-4 antibody was conducted in 3 different mouse syngeneic models GL261, QPP4, and CT-2A. (FIG. 16B) Kaplan-Meier survival curve of GL261 bearing C57BL/6 mice treated with FcE anti-CTLA-4 or parental antibody. (FIG. 16C) Kaplan-Meier survival curve of QPP4 bearing C57BL/6 mice treated with FcE anti-CTLA-4 or parental antibody. (FIG. 16D) Kaplan-Meier survival curve of CT-2A model (left) and tumor rechallenge survival curve from long-term survivors (right). (FIG. 16E) Flow cytometry analysis of immune profiles for comparison of non-treat control tumor and long-term survivors. The myeloid cells and lymphocytes ratio was plotted (left), and CD8/CD4 ratio was plotted (right). (FIG. 16F) Representative CD8 T cell properties were evaluated by PD-1 and IFN-γ expression (left) and a summarized bar graph (right). (FIG. 16G) CD8 immunohistochemistry images of newly developed non-treat control CT-2A tumor (left) and long-term survivor mice (right). (FIG. 16H) Foxp3 immunohistochemistry images of newly developed non-treat control CT-2A tumor (left) and long-term survivor mice (right) (Scale bar=100 μm). Unpaired T-test (FIG. 16E) or 2 way-ANOVA (FIG. 16F) was used for statistical analysis. Data indicate mean±SD, and significance is depicted as ns: not statistically significant, *p<0.05, **p<0.01, ***p<001, ****p<0.0001.

FIGS. 17A-17J: DOX enhances the efficacy of Fc-enhanced anti-CTLA-4 through upregulation of FcγRIIIA/FcγRIV. (FIG. 17A) Clinical course of recurrent GBM patients analyzed in this study. Patients underwent surgery for tumor resection (pre-DOX samples) and skull implantation of the SonoCloud-9 ultrasound device for treatment with previous chemotherapy as described by Sonabend et al., Lancet Oncology 2023. Upon tumoral progression, induction treatment with DOX delivered with LIPU/MB was initiated to treat the recurrent tumor followed by additional treatment cycles with both liposomal DOX and anti-PD-1 delivered by LIPU/MB. The tumor exposed to these therapies was resected (during-DOX samples) and further analyzed. (FIG. 17B) Representative multiplex immunofluorescence images of tumor regions before and during DOX treatment (left; scale bar=1 mm, right; scale bar=50 μm). (FIG. 17C) Percentage of FcγRIIIA positive cells in CD68 positive cells (left) and CD11c positive cells (right) comparing the infiltrating myeloid cells in pre-DOX and during-DOX tumors. Paired T-test was used for statistical analysis. (FIG. 17D) Bar graph showing the concentration of DOX in tumors after 2 days of DOX infusion. (FIG. 17E) Schematic illustration of in vitro DOX effect assay on human microglial cell line (HMC3). (FIG. 17F) Quantitative RT-PCR analysis of FCGR3A expression upon IFN-γ or DOX treatment. (FIGS. 17G and 17H) Flow cytometry analysis showing DOX uptake (left) and FcγRIIIA expression (right) measured as MFI values in HMC3 cells. Unpaired T-test was used for statistical analysis and data indicate mean±SD. (FIG. 171) Kaplan-Meier survival plot of GL261 bearing mice treated with FcE anti-CTLA-4, anti-PD-1/FcE anti-CTLA-4 with and without doxorubicin. (FIG. 17J) Kaplan-Meier curve showing survival of GL261 tumor-bearing mice treated with doxorubicin, anti-PD-1/FcE anti-CTLA-4, and the combination of antibodies and doxorubicin with ultrasound. P-values are provided for Log-rank (Mantel-Cox) test. Statistical significance is depicted as ns: not statistically significant, *p<0.05, **p<0.01, ***p<001, ****p<0.0001.

FIG. 18 demonstrates BBinding kinetics of an Fc-enhanced anti-human CTLA-4 (hIgG1.DLE) or an unmodified human IgG1 variant to human FcγRIIB, FcγRIIIA V158 and FcγRIIIA F158 proteins using SPR. For binding to human FcγRIIIA F158 and V158, antibodies were injected at concentrations ranging from 0.93 to 250 nM. For binding to human FcγRIIB, antibodies were injected at concentrations ranging from 62 nM to 8 μM.

FIG. 19 Demonstrates uniform manifold approximation and projection (UMAP) dimensionality reduction plot indicating cell categories expressing myeloid cell marker (ITGAX (CD11c), CD68, CD163 and FCGR3A) from single-cell RNA sequencing analysis of human recurrent glioblastoma.

FIG. 20 demonstrates quantitative RT-PCR analysis of mouse Fcgrs expression comparing bone marrow-derived macrophages (BMDM), and CT-2A conditioned macrophages (TAM). Unpaired T-test, mean±SD.

FIG. 21 demonstrates a gating strategy for flow cytometry-based analysis.

FIG. 22 demonstrates a peripheral immune response analysis from splenic T cells.

FIG. 23 shows human microglial HMC3 cells express CD68 without IFN-γ treatment and CD163 after IFN-γ treatment.

FIG. 24 shows a general brain toxicity test of the combination therapy.

FIG. 25 Shows a Kaplan-Meier survival curve of QPP4 bearing C57BL/6 mice treated with the combination strategies.

### DETAILED DESCRIPTION

It is to be appreciated that certain aspects, modes, embodiments, variations and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions.

Such modifications and variations are intended to fall within the scope of the appended claims. The present disclosure is to be limited only by the terms of the appended claims, along with the full scope of equivalents to which such claims are entitled.

In practicing the present technologies, many conventional techniques in molecular biology, protein biochemistry, cell biology, microbiology and recombinant DNA are used. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (TRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

Glioblastoma (GBM) remains an incurable disease requiring new treatments and drug delivery modalities. The dire clinical outcomes in GBM patients are explained, at least in part, by the blood-brain barrier (BBB) which can prevent achieving effective concentrations of, for example, systemically administered chemotherapies, antibody-based immunotherapies, and targeted therapies into the brain (Banks, W. A., Nat Rev Drug Discov 15, 275-292 (2016)). Penetration and distribution of certain drugs and biologics in the brain can be improved through opening of the BBB with low intensity pulsed ultrasound (US) in combination with intravenous injection of microbubbles (US/MB or sonication) (Zhang, D. Y., et al., Clin Cancer Res 26, 477-486 (2020); Sabbagh, A., et al., Clin Cancer Res (2021)).

Anthracyclines are drugs extracted from Streptomyces spp. and used to treat various types of cancers. One anthracycline, Doxorubicin (DOX), is a cytotoxic agent that intercalates into the DNA and inhibits the topoisomerase type II, has displayed immunogenic effects in several cancers (Tewey, K. M. et al., Science 226, 466-468 (1984); Kepp, O. et al., Oncoimmunology, 8, e1637188 (2019); Casares, N. et al., J Exp Med, 202, 1691-1701 (2005)). This chemotherapy interestingly promotes the expression of type I interferon (IFN) signature and associated immunogenic cell death in tumor cells (Casares, N. et al., J Exp Med, 202, 1691-1701 (2005); Sistigu, A., et al., Nat Med 20, 1301-1309 (2014)). In addition, these anthracyclines increase the proportion of tumor infiltrating IFNγ⁺ CD8⁺ and CD4⁺ T cells to sustain anticancer activities in preclinical sarcoma, lymphoma, breast, and colon cancer models (Ma, Y. et al., J Exp Med 208, 491-503 (2011); Mattarollo, S. R., et al., Cancer Res 71, 4809-4820 (2011)). The immunological qualities described for DOX have raised interest in combining this chemotherapy with immune checkpoint modulators (e.g., antagonistic antibodies directed to immune checkpoint molecules) to potentiate clinical responses (Kepp, O. et al., Oncoimmunology 8, e1637188 (2019); Galluzzi, L. et al., Nat Rev Clin Oncol 17, 725-741 (2020)). To date, use of anthracyclines, such as DOX, and antibody-based immune checkpoint modulators for the treatment of brain tumors, including GBM, has been limited, at least in part, due to their limited ability to cross the blood-brain barrier.

The present disclosure is based on, among other things, methods of increasing the concentration of anthracyclines, such as DOX, and antibody-based immune checkpoint modulators (e.g., anti-PD-1 and anti-CTLA-4 antibodies) in brain and accordingly, methods of treating brain tumors, such as GBMs. Such technologies provide a solution to the limited ability of anthracyclines (e.g., DOX) and antibody-based immune checkpoint inhibitors (e.g., anti-PD-1 and anti-CTLA-4 antibodies) to cross the blood-brain barrier and provides improved methods of treating brain tumors, including GBMs. In some embodiments, technologies of the present disclosure comprise the discovery and use of methods of increasing the concentration of anthracyclines, antibody-based immune checkpoint modulators (e.g., anti-PD-1 and anti-CTLA-4 antibodies), or any combination thereof in the brain of a subject by administering a therapeutically effective amount of the anthracycline, immune checkpoint modulator, or any combination thereof and disrupting the blood-brain barrier of the subject by administering low-intensity pulsed ultrasound and microbubbles. The disclosure provides, among other things, methods of treating brain tumors (e.g., GBMs) in a subject in need thereof and methods of improving use of immune checkpoint modulators in treating brain tumors (e.g., gliomas and GBMs) comprising administering to the subject a therapeutically effective amount of an anthracycline; administering to the subject a therapeutically effective amount of an immune checkpoint modulator (e.g., anti-PD-1 antibodies) and disrupting the blood-brain barrier of the subject by administering low-intensity pulsed ultrasound and microbubbles.

### Definitions

Unless defined otherwise, all technical and scientific terms used herein have the meaning commonly understood by a person skilled in the art to which this disclosure belongs. The following references provide one of skill with a general definition of many of the terms used in the present disclosure. Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

As used herein, the single forms “a”, “an”, and “the” are intended to include the plural forms as well, unless the context clearly indicates otherwise.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

As used here, the term “about,” when used to modify a numerical value, indicates that deviations of up to 10% above and below the numerical value, including the numerical value, remain within the intended meaning of the recited value. For example, “about 10” should be understood as both “10” and “9-11”.

As used herein, the term “administering” of an agent to a subject includes any route of introducing or delivering the agent to the subject to perform its intended function.

Administration can be carried out by any suitable route, including, but not limited to, intravenously, intramuscularly, intraperitoneally, subcutaneously, and other suitable routes as described herein. Administration includes self-administration and the administration by another.

As used herein, the term “antibody” generally refers to an antibody comprising two light chain polypeptides and two heavy chain polypeptides (unless the context in which this term is used suggests otherwise). Antibodies include different antibody isotypes including IgM, IgG, IgA, IgD, and IgE antibodies. The term “antibody” includes, without limitation, a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, llama, dogs, cats, rabbits, guinea pigs, gerbils, hamsters, rats, and mice. The antibody can be a purified or a recombinant antibody.

An “effective amount” or “efficacious amount” refers to the amount of an agent, or combined amounts of two or more agents, that, when administered for the treatment of a mammal or other subject, is sufficient to effect such treatment for the disease. In some aspects, the effective amount an anthracycline is an amount sufficient to increase or modulate the expression of FcγRIIIA in a subject. In some aspects, the effective amount does not produce a cytotoxic or cardiotoxic effect of anthracyclines. In some aspects, the “effective amount” is sufficient to produce an immune-modulatory effect without producing a cytotoxic or anti-tumor effect. The “effective amount” will vary depending on the agent(s), the disease and its severity and the age, weight, etc., of the subject to be treated. The term “effective amount” refers to a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. With respect to immunogenic compositions, in some embodiments the effective amount will depend on the intended use, the degree of immunogenicity of a particular antigenic compound, and the health/responsiveness of the subject's immune system, in addition to the factors described above. The skilled artisan will be able to determine appropriate amounts depending on these and other factors.

As used herein, the term “blockade” refers to an entity or event whose presence or level correlates with a reduction in level and/or activity of an indicated target. Thus, for example, “a PD-1 blockade” is an agent or event whose presence correlates with reduction in level, activity, and/or antagonism of PD-1. A relevant activity of PD-1 may be or comprise interaction with one of more of its ligands (e.g., PD-L1 and/or PD-L2) and/or a downstream effect thereof. A PD-1 blockade may be achieved by administration of an agent, such as an antibody agent, that targets PD-1 and/or a PD-1 ligand (e.g., PD-L1 and/or PD-L2) and/or a complex thereof. A PD-1 blockade may be achieved through administration of an antibody agent that binds to PD-1 or to PD-L1 (here referred to generically as anti-PD-1). A PD-1 blockade may be achieved through administration of one or more of nivolumab, pembrolizumab, atezolizumab, avelumab, and/or durvalumab. Analogously, a “CTLA-4-blockade” is an agent or event whose presence correlates with reduction in level and/or activity of CTLA-4. A relevant activity of CTLA-4 may be or comprise interaction with one of more of its ligands (e.g., CD80 and/or CD86) and/or a downstream effect thereof. A CTLA-4 blockade may be achieved by administration of an agent, such as an antibody agent, that targets CTLA-4 ligand (e.g., CD80 and/or CD86) and/or a complex thereof. A CTLA-4 blockade may be achieved through administration of an antibody agent that binds to CTLA-4. A CTLA-4 blockade may be achieved through administration of one or more of ipilimumab and/or tremelimumab, or similar antibodies (here referred to anti-CTLA-4 antibodies)

As used herein, the term “combination therapy” refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, the term “effective amount” or “therapeutically effective amount” refers to a quantity of an agent sufficient to achieve a beneficial or desired clinical result upon treatment. In the context of therapeutic applications, the amount of a therapeutic agent administered to the subject can depend on the type and severity of the disease or condition and on the characteristics of the individual, such as general health, age, sex, body weight, effective concentration of the therapeutic agent administered, and tolerance to drugs. It can also depend on the degree, severity, and type of disease. The skilled artisan will be able to determine appropriate dosages depending on these and other factors. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art.

As used herein, the term “immune checkpoint modulator” refers to an agent that interacts directly or indirectly with an immune checkpoint. An immune checkpoint modulator can increase an immune effector response (e.g., cytotoxic T cell response), for example by stimulating a positive signal for T cell activation. An immune checkpoint modulator can increase an immune effector response (e.g., cytotoxic T cell response), for example by inhibiting a negative signal for T cell activation (e.g., disinhibition). An immune checkpoint modulator can interfere with a signal for T cell anergy. An immune checkpoint modulator can reduce, remove, and/or or prevent immune tolerance to one or more antigens.

As used herein, the term “reduce” or “decrease” means to alter negatively by at least about 5% including, but not limited to, alter negatively by about 5%, by about 10%, by about 25%, by about 30%, by about 50%, by about 75%, or by about 100%.

As used herein, the terms “specific binding,” “specifically binds,” “selective binding,” and “selectively binds,” are intended to mean that an antibody or antigen-binding fragment thereof that exhibits appreciable affinity for a particular antigen or ligand (e.g., an immune checkpoint target described herein) and, generally, does not bind to, or substantially does not bind to, other antigens or ligands. “Appreciable” or preferred binding includes binding with a K_{D} of 10⁷, 10⁸, 10⁹, or 10¹⁰ M or better. The K_{D} of an antibody or antigen-binding fragment-antigen or ligand interaction (the affinity constant) indicates the concentration of antibody or antigen-binding fragment at which 50% of antibody or antigen-binding fragment and antigen or ligand molecules are bound together. Thus, at a suitable fixed antigen concentration, 50% of a higher (i.e., stronger) affinity antibody or antigen-binding fragment will bind antigen or ligand molecules at a lower antibody or antigen-binding fragment concentration than would be required to achieve the same percent binding with a lower affinity antibody or antigen-binding fragment. Thus, a lower K_{D} value indicates a higher (stronger) affinity. As used herein, “better” affinities are stronger affinities, and are of lower numeric value than their comparators, with a K_{D} of 10⁷ M being of lower numeric value and therefore representing a better affinity than a K_{D} of 10⁶ M. Affinities better (i.e., with a lower K_{D} value and therefore stronger) than 10⁷ M, preferably better than 10⁸ M, are generally preferred. Values intermediate to those set forth herein are also contemplated, and a preferred binding affinity can be indicated as a range of affinities.

In certain embodiments, the terms “disease” “disorder” and “condition” are used interchangeably herein, referring to a cancer, a status of being diagnosed with a cancer, or a status of being suspect of having a cancer.

As used herein, a “cancer” is a disease state characterized by the presence in a subject of cells demonstrating abnormal uncontrolled replication and may be used interchangeably with the term “tumor.” In some embodiments, the cancer is a glioma or glioblastoma. “Cell associated with the cancer” refers to those subject cells that demonstrate abnormal uncontrolled replication.

“Cancer”, which is also referred to herein as “tumor”, is a known medically as an uncontrolled division of abnormal cells in a part of the body, benign or malignant. In one embodiment, cancer refers to a malignant neoplasm, a broad group of diseases involving unregulated cell division and growth, and invasion to nearby parts of the body. Non-limiting examples of cancers include carcinomas, sarcomas, leukemia and lymphoma, e.g., colon cancer, colorectal cancer, rectal cancer, gastric cancer, esophageal cancer, head and neck cancer, breast cancer, brain cancer, lung cancer, stomach cancer, liver cancer, gall bladder cancer, or pancreatic cancer. In one embodiment, the term “cancer” refers to a solid tumor, which is an abnormal mass of tissue that usually does not contain cysts or liquid areas, including but not limited to, sarcomas, carcinomas, and certain lymphomas (such as Non-Hodgkin's lymphoma). In another embodiment, the term “cancer” refers to a liquid cancer, which is a cancer presenting in body fluids (such as, the blood and bone marrow), for example, leukemias (cancers of the blood) and certain lymphomas.

Additionally or alternatively, a cancer may refer to a local cancer (which is an invasive malignant cancer confined entirely to the organ or tissue where the cancer began), a metastatic cancer (referring to a cancer that spreads from its site of origin to another part of the body), a non-metastatic cancer, a primary cancer (a term used describing an initial cancer a subject experiences), a secondary cancer (referring to a metastasis from primary cancer or second cancer unrelated to the original cancer), an advanced cancer, an unresectable cancer, or a recurrent cancer. As used herein, an advanced cancer refers to a cancer that had progressed after receiving one or more of. the first line therapy, the second line therapy, or the third line therapy.

A “solid tumor” is an abnormal mass of tissue that usually does not contain cysts or liquid areas. Solid tumors can be benign or malignant. Different types of solid tumors are named for the type of cells that form them. Examples of solid tumors include sarcomas, carcinomas, and lymphomas. The solid tumor can be localized or metastatic.

As used herein, “monoclonal antibody” refers to an antibody obtained from a substantially homogeneous antibody population. Monoclonal antibodies are highly specific, as each monoclonal antibody is directed against a single determinant on the antigen. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

Monoclonal antibodies may be generated using hybridoma techniques or recombinant DNA methods known in the art. A hybridoma is a cell that is produced in the laboratory from the fusion of an antibody-producing lymphocyte and a non-antibody producing cancer cell, usually a myeloma or lymphoma. A hybridoma proliferates and produces a continuous sample of a specific monoclonal antibody. Alternative techniques for generating or selecting antibodies include in vitro exposure of lymphocytes to antigens of interest, and screening of antibody display libraries in cells, phage, or similar systems.

The term “human antibody” as used herein, is intended to include antibodies having variable and constant regions derived from human germline immunoglobulin sequences. The human antibodies disclosed herein may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

A “human monoclonal antibody” refers to antibodies displaying a single binding specificity which have variable and constant regions derived from human germline immunoglobulin sequences. The term also intends recombinant human antibodies. Methods to making these antibodies are described herein.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. Methods to making these antibodies are described herein.

As used herein, chimeric antibodies are antibodies whose light and heavy chain genes have been constructed, typically by genetic engineering, from antibody variable and constant region genes belonging to different species.

As used herein, the term “humanized antibody” or “humanized immunoglobulin” refers to a human/non-human chimeric antibody that contains a minimal sequence derived from non-human immunoglobulin. For the most part, humanized antibodies are human immunoglobulins (recipient antibody) in which residues from a variable region of the recipient are replaced by residues from a variable region of a non-human species (donor antibody) such as mouse, rat, rabbit, or non-human primate having the desired specificity, affinity and capacity. Humanized antibodies may comprise residues that are not found in the recipient antibody or in the donor antibody. The humanized antibody can optionally also comprise at least a portion of an immunoglobulin constant region (Fc), typically that of a human immunoglobulin, a non-human antibody containing one or more amino acids in a framework region, a constant region or a CDR, that have been substituted with a correspondingly positioned amino acid from a human antibody. In general, humanized antibodies are expected to produce a reduced immune response in a human host, as compared to a non-humanized version of the same antibody. The humanized antibodies may have conservative amino acid substitutions which have substantially no effect on antigen binding or other antibody functions. Conservative substitutions groupings include: glycine-alanine, valine-leucine-isoleucine, phenylalanine-tyrosine, lysine-arginine, alanine-valine, serine-threonine and asparagine-glutamine. The antibodies of the present invention may bind to an antigen or molecule. In some aspects, the antigens or molecules are expressed in a T cell, tumor cell, or tissue of a subject.

The terms “polyclonal antibody” or “polyclonal antibody composition” as used herein refer to a preparation of antibodies that are derived from different B-cell lines. They are a mixture of immunoglobulin molecules secreted against a specific antigen, each recognizing a different epitope.

As used herein, the term “antibody derivative”, comprises a full-length antibody or a fragment of an antibody, wherein one or more of the amino acids are chemically modified by alkylation, pegylation, acylation, ester formation or amide formation or the like, e.g., for linking the antibody to a second molecule. This includes, but is not limited to, pegylated antibodies, cysteine-pegylated antibodies, and variants thereof.

### Anthracyclines

Anthracyclines are a family of antibiotics often utilized in cancer chemotherapy. The anthracycline group of compounds, and its pharmaceutically acceptable salts and derivatives, can be broadly defined as comprising a planar anthraquinone chromophore that can intercalate between adjacent base pairs of DNA. This chromophore is generally linked to a duanosamine sugar moiety. Anthracyclines, as used herein, refers to both naturally-occurring anthracyclines, such as those isolated from bacterial species, but also encompasses synthetic and semisynthetic derivatives. Compounds belonging to the anthracycline group of compounds include, for example and without limitation, daunorubicin, doxorubicin, epirubicin, idarubicin, mitoxantrone, berubicin, annamycin and their pharmaceutically acceptable salts.

Daunorubicin (DNR) is understood to function through multiple mechanisms to mediate DNA damage. Interaction of the duanosamin residue into the minor groove of cellular DNA leads to local DNA unwinding and it is hypothesized that these DNA-DNR complexes serve as a blockade for cellular replication. The activity of topoisomerase II, an enzyme that relives torsional stress during DNA synthesis, is also significantly inhibited by DNR. Additionally, cellular metabolism of DNR leads to the formation of free radical species capable of inducing DNA damage. Without wishing to be bound by any one theory, it is thus understood that, through the non-specific targeting of DNA replication, DNR targets all cells with a high proliferation index. See, e.g., Petre C E et al., Int J Nanomedicine. 2007; 2(3):277-88).

Idarubicin is a DNA-intercalating analog of DNR which has an inhibitor effect on nucleic acid synthesis and interacts with the enzyme, topoisomerase II (Idarubicin hydrochloride for injection, USP: Pfizer; 2006).

Doxorubicin (DOX) is a cytotoxic anthracycline that intercalates into the DNA and inhibits the topoisomerase type II (Tewey, K. M. et al., Science 226, 466-468 (1984)) and has displayed immunogenic effects in several cancers (Kepp, O. et al., Oncoimmunology 8, e1637188 (2019); Casares, N. et al., J Exp Med 202, 1691-1701 (2005)). DOX also promotes the expression of the type I interferon (IFN) signature and associated immunogenic cell death in tumor cells (Casares, N. et al., J Exp Med 202, 1691-1701 (2005); Sistigu, A. et al., Nat Med 20, 1301-1309 (2014)). In addition, these anthracyclines increase the proportion of tumor infiltrating IFN-γ⁺ CD8⁺ and CD4⁺ T cells to sustain anticancer activities in preclinical sarcoma, lymphoma, breast, and colon cancer models (Ma, Y. et al., J Exp Med 208, 491-503 (2011); Mattarollo, S. R., et al., Cancer Res 71, 4809-4820 (2011)).

Epirubicin is an epimer of doxorubicin. Epirubicin is understood to function through intercalation of DNA, inhibition of topoisomerase II activity, and generation of oxygen and drug free radical with consequence interference with DNA, RNA, and protein synthesis (Khasraw, M. et al., The Breast Vol. 21(2), P 142-149 (2012)).

Mitoxantrone is an antineoplastic antibiotic that is a synthetic derivative of DOX and is considered an anthracenedione. It is understood to act by intercalating into helical double-stranded DNA causing cross links and strand breaks, thus blocking both DNA and RNA synthesis (LiverTox: Clinical and Research Information on Drug-Induced Liver Injury. Bethesda (MD): National Institute of Diabetes and Digestive and Kidney Diseases; 2012-. Mitoxantrone).

Berubicin (also known as WP744) is a doxorubicin analog that is understood to cross the blood brain barrier and have significant central nervous system uptake. Induction of apoptosis and DNA damage by berubicin has been compared to DOX and can show increased potency and cytotoxicity relative to DOX. See, e.g., Silberman, S. et al., Journal of Clinical Oncology, 2022 40:16_suppl, TPS2083-TPS2083).

Annamycin (3′-deamino-4′-epi-3′hydroxy-2′-iodo-4-demethoxydoxorubicin) is a highly lipophilic form of the anthracycline doxorubicin with the ability to bypass the ABCB1 mechanism of cellular drug resistance. Annamycin is also understood to have an increased affinity to liposomes which can improve drug targeting and reduce cardiac toxicity. See, e.g., Wetzler M. et al., Clin Lymphoma Myeloma Leuk. 2013 August; 13(4):430-4).

Liposome-encapsulated anthracyclines (e.g., liposomal DOX, liposomal DNR) were designed to reduce the toxicity of such anthracyclines while preserving their antitumor efficacy by altering its tissue distribution and pharmacokinetics. Without wishing to be bound by any one theory, it is understood that intravenously injected liposomes cannot escape the vascular space in sites that have tight capillary junctures, such as the heart muscle and gastrointestinal tract. Liposomes generally exit the circulation in tissues and organs lines with cells that are not tightly joined (e.g., fenestrated) or areas where the capillaries are disrupted by inflammation or tumor group. Thus, it is understood liposomal formulation of anthracyclines directs the anthracycline away from sites of potential toxicity (e.g., the heart), but leaves tumors exposed to the drug.

Anthracyclines, including liposomal anthracylines, can also be pegylated (e.g., polyethylene glycol coated). Pegylated liposomal anthracyclines (e.g., pegylated DOX) is a formulation of anthracycline in which the molecule is packaged in a liposome with an outer coating of polyethylene glycol. The polyethylene glycol coating on the liposomes creates a hydrophilic layer around the liposome that buffers the liposomal wall from the surrounding milieu. Without wishing to be bound by any one theory, it is understood that this coating decreases proteins from binding to the lipid bilayer. Such proteins act as opsonins, attracting foreign particles that in turn activate the mononuclear phagocytic cells which leads to the breakdown of the liposome and release of the drug.

In one aspect, the present disclosure provides methods for treating a glioblastoma in a subject in need thereof comprising administering to the subject a therapeutically effective amount of an anthracycline. In some such embodiments, the anthracycline is or comprises one or more of daunorubicin, doxorubicin, epirubicin, idarubicin, mitoxantrone, berubicin, annamycin and their pharmaceutically acceptable salts. In some such embodiments, the anthracycline is or comprises liposomal anthracycline, such as liposomal DOX or liposomal DNR. In some such embodiments, the anthracycline is or comprises a pegylated anthracycline, including, for example, pegylated liposomal DOX.

As will be readily apparent by one of ordinary skill in the art, the type of cancer and its stage, among other things (e.g., subject body weight, metabolism) can influence the dose and duration of anthracycline administration.

Anthracyclines are generally administered by injection (e.g., intramuscularly) or by intravenous infusion. In some embodiments, anthracyclines for use in accordance with technologies of the present disclosure are administered by intramuscular injection. In some embodiments, anthracyclines for use in accordance with technologies of the present disclosure are administered by intravenous infusion. Anthracyclines can also be administered via convention-enhanced delivery (see, e.g., Saito R et al., Neurol Med Chir (Tokyo). 2017 Jan. 15; 57(1):8-16) or by intravenous injection with subsequent laser-interstitial thermal therapy (see, e.g., Salehi A et al., Neurooncol Adv. 2020 Jun. 30; 2(1)). In some embodiments, anthracyclines for use in accordance with technologies of the present disclosure are administered by convection-enhanced delivery. In some embodiments, anthracyclines for use in accordance with technologies of the present disclosure are administered by intravenous injection with subsequent laser-interstitial thermal therapy.

In some embodiments, a therapeutically effective amount of an anthracycline is a non-cytotoxic dose. In some embodiments, a therapeutically effective amount of an anthracycline is about 10 mg/m², 15 mg/m², 20 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², or 80 mg/m². In some embodiments, a therapeutically effective amount of an anthracycline is about 10 mg/m²-80 mg/m², 10 mg/m²-70 mg/m², 10 mg/m²-60 mg/m², 10 mg/m²-50 mg/m², 10 mg/m²-40 mg/m², 20 mg/m²-80 mg/m², 20 mg/m²-70 mg/m², 20 mg/m²-60 mg/m², 20 mg/m²-50 mg/m², 20 mg/m²-50 mg/m², 30 mg/m²-80 mg/m², 30 mg/m²-70 mg/m², 30 mg/m²-60 mg/m², 30 mg/m²-50 mg/m², or 20 mg/m²-40 mg/m².

In some embodiments, an anthracycline is administered at a frequency of about every week, every 2 weeks, every 3 weeks, every 4 weeks, every 5 weeks, or every 6 weeks. In some such embodiments, DOX (e.g., liposomal DOX, pegylated liposomal DOX) is administered in therapeutically effective amount of 30 mg every three weeks.

### Immune Checkpoint Modulators

An immune checkpoint refers to inhibitory pathways of an immune system that are responsible for maintaining self-tolerance and modulating duration and amplitude of physiological immune responses. Certain cancer cells thrive by taking advantage of immune checkpoint pathways as a major mechanism of immune resistance, particularly with respect to T cells that are specific for tumor antigens. For example, certain cancer cells may overexpress one or more immune checkpoint proteins responsible for inhibiting a cytotoxic T cell response. Thus, among other things, immune checkpoint modulators (e.g., as described herein) may be administered to overcome inhibitory signals and permit and/or augment an immune attack against cancer cells. Immune checkpoint modulators may facilitate immune cell responses against cancer cells by decreasing, inhibiting, and/or abrogating signaling by negative immune response regulators (e.g., CTLA-4) or may stimulate or enhance signaling of positive regulators of immune response (e.g., CD28).

An immune checkpoint modulator refers to an agent that interacts directly or indirectly with an immune checkpoint. In some embodiments, an immune checkpoint modulator increases an immune effector response (e.g., cytotoxic T cell response), for example by stimulating a positive signal for T cell activation. In some embodiments, an immune checkpoint modulator increases an immune effector response (e.g., cytotoxic T cell response), for example by inhibiting a negative signal for T cell activation (e.g., disinhibition). In some embodiments, an immune checkpoint modulator interferes with a signal for T cell anergy. In some embodiments, an immune checkpoint modulator reduces, removes, or prevents immune tolerance to one or more antigens.

In some embodiments, the present disclosure, among other things, relates to administration of an immune checkpoint modulator to a subject in need thereof (e.g., a subject with a brain cancer such as glioblastoma). In some embodiments, administration of an immune checkpoint modulator comprises administration of one or more immune checkpoint modulators. In some embodiments, an immune checkpoint modulator is an agent (e.g., an antibody) that targets (i.e., specifically binds to) an immune checkpoint target. In some embodiments, an immune checkpoint target is or comprises one or more of CTLA-4, PD-1, PD-L1, GITR, OX40, LAG-3, KIR, TIM-3, CD28, CD40, and CD137. In some such embodiments, an immune checkpoint modulator is or comprises one or more of an anti-CTLA-4 antibody, an anti-PD-1 antibody, an anti-PD-L1 antibody, an anti-GITR antibody, an anti-OX40 antibody, an anti-LAG-3 antibody, an anti-KIR antibody, an anti-TIM-3 antibody, an anti-CD28 antibody, an anti-CD40 antibody, an anti-CD-137 antibody, or any combination thereof.

A plurality of immune checkpoint modulators are known in the art. In some embodiments, an immune checkpoint modulator is or comprises an anti-CTLA-4 antibody. In some such embodiments, an immune checkpoint modulator is selected from the group consisting of: tremelimumab, ipilimumab, zalifrelimab, and any combination thereof. In some embodiments, an anti-CTLA-4 antibody is or comprises tremelimumab. In some embodiments, an anti-CTLA-4 antibody is or comprises ipilimumab. In some embodiments, an anti-CTLA-4 antibody is or comprises zalifrelimab.

In some embodiments, an immune checkpoint modulator is or comprises an anti-PD-1 antibody. In some such embodiments, an anti-PD-1 antibody is selected from the group consisting of: nivolumab, pembrolizumab, balstilimab, cemiplimab, and any combination thereof. In some embodiments, an anti-PD-1 antibody is or comprises nivolumab. In some embodiments, an anti-PD-1 antibody is or comprises pembrolizumab. In some embodiments, an anti-PD-1 antibody is or comprise balstilimab. In some embodiments, an anti-PD-1 antibody is or comprises cemiplimab.

In some embodiments, an immune checkpoint modulator is or comprises an anti-PD-L1 antibody. In some such embodiments, an anti-PD-L1 antibody is selected from the group consisting of: atezolizumab, avelumab, durvalumab, and any combination thereof. In some embodiments, an anti-PD-L1 antibody is or comprises atezolizumab. In some embodiments, an ani-PD-L1 antibody is or comprises avelumab. In some embodiments, an ani-PD-L1 antibody is or comprises durvalumab.

As will be readily apparent by one of ordinary skill in the art, the type of cancer and its stage, among other things (e.g., subject body weight, metabolism) can influence the dose and duration of immune checkpoint modulator administration.

Immune checkpoint modulators are generally administered intravenous infusion. In some embodiments, immune checkpoint modulators for use in accordance with technologies of present disclosure are administered by intravenous infusion.

In some embodiments, a therapeutically effective amount of an immune checkpoint modulator is about 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg. In some embodiments, a therapeutically effective amount of an immune checkpoint modulator is about 1-20 mg/kg, 2-20 mg/kg, 3-20 mg/kg, 4-20 mg/kg, 5-20 mg/kg, 6-20 mg/kg, 7-20 mg/kg, 8-20 mg/kg, 9-20 mg/kg, 10-20 mg/kg, 1-15 mg/kg, 2-15 mg/kg, 3-15 mg/kg, 4-15 mg/kg, 5-15 mg/kg, 1-10 mg/kg, 2-10 mg/kg, 3-10 mg/kg, 4-10 mg/kg, or 5-12 mg/kg.

In some embodiments, an immune checkpoint modulator is administered at a frequency of about every 1 day, every 2 days, every 3 days, every 4 days, every 5 days, every 6 days, every 1 week, every 2 weeks, every 3 weeks, every 4 weeks, every 5 weeks, or every 6 weeks.

### Disruption of Blood-Brain Barrier (BBB)

In one aspect, the present disclosure provides methods of disrupting the blood-brain barrier (BBB) to increase the concentration of an anthracycline, an immune checkpoint modulator, or any combination thereof in the brain of a subject and methods of treating a glioblastoma in a subject in need thereof comprising disruption of the BBB. The BBB is a component of the neurovascular unit (NVU) and acts as the blood-brain interface mediating communication between the central nervous system (CNS) and the periphery. The BBB is a physiologic barrier comprising a monolayer of endothelial cells connected to each other by means of a tight junction. The BBB operates by using active and passive transport mechanisms to limit the passage of potentially toxic molecules from the blood to the brain. Only lipophilic molecules with molecular weights of less than about 400 daltons readily cross the BBB. Accordingly, approximately 98% of small-molecule therapeutics and 100% of large-molecule therapeutics do not cross the intact BBB. As such, the penetration of small- and large-molecule therapeutics into the brain, and subsequently brain tumors (e.g., GBMs), and in particular infiltrative tumor cells, is limited.

A plurality of methods to enhance penetration of small- and large-molecule therapeutics have been tested, such as convection-enhanced delivery (CED) and intra-arterial (IA) delivery, but have failed to gain clinical adoption at least because of the difficulty of repeated use, limitations of area and time frame of therapeutic delivery, and/or associated complications. One method to enhance the delivery of therapeutics into the brain is to temporarily increase the permeability of the BBB. Without wishing to be bound by any one theory, when the permeability of the BBB is temporarily increased, the passage of therapeutics from the blood to the cerebral parenchyma can be enhanced. One such method to temporarily increase the permeability of the BBB is the use of ultrasound. It is understood that the intensity of ultrasound required to increase permeability of the BBB can be significantly reduced, and repeatability and safety increased, if low-intensity ultrasound is administered in combination with injection of microbubbles. In some embodiments, methods of the present disclosure disrupt the BBB by administering low-intensity pulsed ultrasound (LIPUS) and microbubbles.

Low-intensity pulsed ultrasound (LIPUS) is a specific type of ultrasound that delivers at a low intensity and outputs in the mode of pulsed waves. It has minimal thermal effects (e.g., does not significantly increase biological temperature, in contrast to high-intensity ultrasound which can be used for tissue heating), while maintaining the transmission of acoustic energy to the target tissue, which can provide minimally- or non-invasive physical stimulation for therapeutic applications. Without wishing to be bound by any one theory, BBB disruption by LIPUS is understood to result from mechanical interaction of the ultrasound wave and circulating microbubbles. When ultrasound stimulates circulating microbubbles, the bubbles expand and contract, resulting in mechanical stretch of the vessel wall when the size of the microbubble is similar to the diameter of the capillary lumen.

LIPUS can be administered using an ultrasound device. In some embodiments, an ultrasound device is surgically implanted into the skull of a subject in need thereof. In some embodiments, an ultrasound device is implanted in a cranial window in the skull of the subject in need thereof. Without wishing to be bound by any one theory, direct administration of LIPUS to the brain tissue prevents distortion and/or attenuation of the energy by the skull bone. Accordingly, it is understood that this approach allows for precise administration of the ultrasound energy (e.g., LIPUS) and pressure delivered to the brain tissue, and sonication of a large volume.

In some embodiments, LIPUS is administered as focused ultrasound. In some embodiments, LIPUS is administered as unfocused ultrasound.

LIPUS can be generated with a constant acoustic pressure. In some embodiments, the constant acoustic pressure is about 0.90 megapascals (MPa), 0.91 MPa, 0.92 MPa, 0.93 MPa, 0.94 MPa, 0.95 MPa, 0.96 MPa, 0.97 MPa, 0.98 MPa, 0.99 MPa, 1.00 MPa, 1.01 MPa, 1.02 MPa, 1.03 MPa, 1.04 MPa, 1.05 MPa, 1.06 MPa, 1.07 MPa, 1.08 MPa, 1.09 MPa, or 1.10 MPa. In some embodiments, the constant acoustic pressure is about 0.90-1.10 MPa, 0.95-1.10 MPa, 0.90-1.05 MPa, 0.95-1.05 MPa, 0.98-1.10 MPa, 0.98-1.05 MPa, 0.98-1.03 MPa, 1.00-1.10 MPa, or 1.03-1.10 MPa.

LIPUS can be administered at a plurality of frequencies. In some embodiments, the LIPUS is administered at a frequency of about 200 kilohertz (kHz), 225 kHz, 250 kHz, 275 kHz, 300 kHz, 325 kHz, 350 kHz, 375 kHz, 400 kHz, 425 kHz, 450 kHz, 475 kHz, 500 kHz, 525 kHz, 550 kHz, 575 kHz, 600 kHz, 625 kHz, 650 kHz, 675 kHz, 700 kHz, 725 kHz, 750 kHz, 775 kHz, 800 kHz, 825 kHz, 850 kHz, 875 kHz, 900 kHz, 925 kHz, 950 kHz, 975 kHz, 1 mHz, 2 mHz, 3 mHz, 4 mHz, 5 mHz, 6 mHz, 7 mHz, 8 mHz, 9 mHz, or 10 mHz. In some embodiments, the LIPUS is administered at a frequency of about 200 kHz-10 mZ, 200 kHz-8 mHz, 200 kHz-1 mHz, 220 kHz-10 mHz, 220 kHz-8 mHz, 220 kHz-1 mHz, 400 kHz-10 mHz, 400 kHz-8 mHz, 400 kHz-1 mHz, 750 kHz-10 mHz, 750 kHz-8 mHz, or 750 kHz-1 mHz.

LIPUS can be administered according to a plurality of pulse conditions. Pulses can be administered for a plurality of durations and at a plurality of frequencies over a period of time. In some embodiments, LIPUS is administered at a pulse duration of at least about 5 milliseconds (ms), 10 ms, 15 ms, 20 ms, 25 ms, 30 ms, 35 ms, 40 ms, 45 ms, 50 ms, 75 ms, 100 ms or more. In some embodiments, LIPUS is administered at a pulse duration of about 5 ms-100 ms, 10 ms-100 ms, 15 ms-100 ms, 20 ms-100 ms, 25 ms-100 ms, 30 ms-100 ms, 50 ms-100 ms, 5 ms-75 ms, 10 ms-75 ms, 20 ms-75 ms, 15 ms-50 ms, or 20 ms-30 ms. In some embodiments, LIPUS is administered at a pulse frequency of about every 1 second, every 2 seconds, every 3 seconds, every 4 seconds, every 5 seconds, every 6 seconds, every 7 seconds, every 8 seconds, every 9 seconds, every 10 seconds, every 12 seconds, every 15 seconds, every 20 seconds, every 25 seconds, or every 30 seconds. In some embodiments, LIPUS is administered over a period of time of about 120 seconds, 150 seconds, 180 seconds, 210 seconds, 240 seconds, 270 seconds, 300 seconds, 330 seconds, 360 seconds, 390 seconds, 420 seconds, or 450 seconds.

In some embodiments, methods of disrupting the blood brain barrier may enhance the effectiveness of anthracyclines. However, while such methods may provide some assist in the permeability of the blood brain barrier, they are not necessary for the effective modulation of FcγRIIIA via the administration of anthracyclines. In some embodiments, LIPUS is administered prior to administration of microbubbles. In some embodiments, LIPUS is administered simultaneously to administration of microbubbles. In some embodiments, LIPUS is administered to following administration of microbubbles. In some embodiments, microbubbles are administered by intravenous injection. In some embodiments, microbubbles are administered by systemic injection.

In some embodiments, microbubbles are about 1 μm in diameter. In some embodiments, microbubbles are about 2 μm in diameter. In some embodiments, microbubbles are about 3 μm in diameter. In some embodiments, microbubbles are about 1-3 μm in diameter.

In some embodiments, microbubbles are administered (e.g., by intravenous injection) as a bolus. In some embodiments, microbubbles are administered over a period of time. In some such embodiments, the period of time is about 10 seconds, 15 seconds, 20 seconds, 25 seconds 30 seconds, 35 seconds, 40 seconds, 45 seconds, 50 seconds, 55 seconds, or 60 seconds. In some such embodiments, the period of time is about 10-60 seconds, 15-60 seconds, 20-60 seconds, 25-60 seconds, 30-60 seconds, 10-50 seconds, 15-50 seconds, 20-50 seconds, 20-40 seconds, 20-30 seconds, 15-45 seconds, or 15-35 seconds.

In some embodiments, microbubbles are administered at a dose of about 1 μL/kg, 2 μL/kg, 5 μL/kg, 7 μL/kg, 10 μL/kg, 12 μL/kg, 15 μL/kg, 17 μL/kg, or 20 μL/kg. In some embodiments, microbubbles are administered at a dose of about 1-20 μL/kg, 2-20 μL/kg, 5-20 μL/kg, 7-17 μL/kg, 7-15 μL/kg, or 7-12 μL/kg.

In some embodiments, microbubble administration is followed by a saline flush. In some such embodiments, a saline flush is administered with a particular volume over a period of time. In some embodiments, a saline flush of at least about 10 mL is administered (including, e.g., 12 mL, 15 mL, 20 mL or more). In some embodiments, a saline flush is administered over a period of time of about 20 seconds, 30 seconds, 40 seconds, 50 seconds, 60 seconds, or more.

### Therapeutic Methods and Uses

In one aspect, the present disclosure provides a method for treating cancer in a subject in need thereof comprising, consisting of, or consisting essentially of administering to the subject a therapeutically effective amount of an anthracycline. In some aspects, the method further comprises, consists of, or consists essentially of administering to the subject a therapeutically effective amount of an checkpoint inhibitor. In some aspects, the checkpoint inhibitor comprises consists of or consists essentially of an anti-CTLA-4 antibody. In some aspects, the method further comprises, consists of, or consists essentially of administering to the subject a therapeutically effective amount of an anti-PD-1 antibody. In some aspects, the method comprises, consists of, or consists essentially of disrupting the blood-brain barrier of the subject by administering low-intensity pulsed ultrasound and microbubbles.

In some embodiments, the effective amount of the anthracycline is below the established human cytotoxic or cardiotoxic amount a sub-therapeutic amount. In some embodiments, the therapeutically effective amount of the anthracycline comprises 30 mg or less. In some aspects, the effect amount of the anthracycline comprises 35 mg or less, or 40 mg or less, or 45 mg or less, or 50 mg or less. In some embodiments, the therapeutically effective amount of the anthracycline comprises an amount that results in a tumor concentration of at least 0.1 mM of anthracycline 0.2 mM or greater, 0.3 mM or greater, or 0.4 mM or greater, or 0.5 mM or greater. In some embodiments, the therapeutically effective amount of the anthracycline comprises an amount that results in a blood serum concentration of at least 0.1 mM of anthracycline, 0.2 mM or greater, 0.3 mM or greater, or 0.4 mM or greater, or 0.5 mM or greater. In some aspects the effective amount of the anthracycline does not result in cytotoxicity in the subject. In some aspects, the effective amount of the anthracycline is a non-cytotoxic dose of an anthracycline. In some aspects, the effective amount of the anthracycline is sufficient to increase expression of FcγRIIA in the subject.

In some aspects, the therapeutically effective amount of the anthracycline and the therapeutically effective amount of the checkpoint inhibitor are administered simultaneously. In other aspects, the therapeutically effective amount of anthracycline is administered immediately after the therapeutically effective amount of the checkpoint inhibitor is administered. In some aspects, the therapeutically effective amount of anthracycline is administered before the therapeutically effective amount of the checkpoint inhibitor antibody is administered. In some aspects, there is a period of time between administering the therapeutically effective amount of anthracycline and therapeutically effective amount of the checkpoint inhibitor, wherein the period of time comprises, consists of, or consists essentially of about 1 to about 8 hours.

In one aspect, the present disclosure provides methods for treating a brain tumor (e.g., a glioblastoma) in a subject in need thereof comprising (a) administering to the subject a therapeutically effective amount of an anthracycline; (b) administering to the subject a therapeutically effective amount of an immune checkpoint modulator; and (c) disrupting the blood-brain barrier of the subject by administering low-intensity pulsed ultrasound and microbubbles. In some embodiments, the therapeutically effective amount of the anthracycline (e.g., DOX) and the low-intensity pulsed ultrasound and microbubbles are administered simultaneously. In some embodiments, the therapeutically effective amount of the anthracycline (e.g., DOX), the therapeutically effective amount of the immune checkpoint modulator (e.g., anti-PD-1 antibody), and the low-intensity pulsed ultrasound and microbubbles are administered simultaneously. In some embodiments, the therapeutically effective amount of anthracycline (e.g., DOX) is administered immediately after the low-intensity pulsed ultrasound and microbubbles are administered. In some embodiments, there is a period of time between administering the therapeutically effective amount of the immune checkpoint modulator (e.g., anti-PD-1 antibody) and administering the low-intensity pulsed ultrasound and microbubbles. In some such embodiments, the period of time is at least about 30 minutes, about 1 hour, about 2 hours, about 3 hours, about 4 hours, about 5 hours, about 6 hours, about 7 hours, about 8 hours, about 9 hours, about 10 hours, about 12 hours, about 1 day, about 2 days, about 3 days, about 4 days, about 5 days, about 6 days, about 7 days, about 14 days, or about 21 days. In some such embodiments, the period of time is about 30 minutes to about 12 hours, about 30 minutes to about 8 hours, about 30 minutes to about 6 hours, about 30 minutes to about 4 hours, about 1 hour to about 12 hours, about 1 hour to about 10 hours, about 1 hour to about 8 hours, about 1 hour to about 6 hours, about 1 day to about 21 days, about 1 day to about 14 days, about 1 day to about 7 days, or about 1 day to about 4 days.

In one aspect, the present disclosure provides technologies for preventing cancer in a subject in remission from brain cancer (e.g., glioblastoma), comprising administering to the subject in need thereof (a) a therapeutically effective amount of anthracycline (e.g., DOX), (b) a therapeutically effective amount of an immune checkpoint modulator (e.g., anti-PD-1 antibody), and (c) low-intensity pulsed ultrasound and microbubbles.

In some embodiments, the brain cancer (e.g., glioblastoma) is a relapsed cancer. In some embodiments, the brain cancer (e.g., glioblastoma) is a refractory cancer. In some embodiments, the brain cancer (e.g., glioblastoma) is a recurrent cancer. In some embodiments, the brain cancer (e.g., glioblastoma) is an advanced stage cancer. In some embodiments, the brain cancer (e.g., glioblastoma) is resistant to one or more other therapies (e.g., chemotherapy, radiotherapy, stem cell transplantation, or another immunotherapy).

The effectiveness of any therapy described herein can be assessed by evaluating a parameter (e.g., tumor burden) before and after administration of the therapy (e.g., to the subject in need thereof). Any assay known in the art can be used to evaluate the therapeutic effectiveness of the therapies described herein.

In one aspect, technologies of the present disclosure provide methods of increasing the concentration of an anthracycline, an immune checkpoint modulator, or any combination thereof in the brain of a subject comprising: (a) administering a therapeutically effective amount of the anthracycline, the immune checkpoint modulator, or any combination thereof to the subject, and (b) disrupting the blood-brain barrier of the subject by administering low-intensity pulsed ultrasound and microbubbles.

While some aspects of the disclosure include disrupting the blood brain barrier to increase the effectiveness an uptake of an anthracycline, such aspects of the disclosure are not essential to result in a therapeutic effect. Specifically, and by way of experimental example, the methods of the instant disclosure demonstrate that low-doses of anthracyclines are effect at modulating and/or up-regulating the expression of FcγRIIA and improving a subjects response to immune checkpoint inhibitors. These effects are applicable cancers susceptible to treatment with immune checkpoint inhibitors, including, but not limited to, primary brain cancers. The effects are particularly useful for immune checkpoint inhibitors that rely upon Fc region binding on immune cells (e.g. myeloid cells or NK cells) for activity.

In one aspect, technologies of the present disclosure provide methods of improving immune checkpoint modulator-based therapy, and in particular, for brain cancers, including glioblastomas.

### Patient Populations

In some embodiments, a subject in need thereof in accordance with the technologies disclosed herein include, but are not limited to, humans and non-human vertebrates. In some embodiments, a subject in need thereof in accordance with the technologies disclosed herein comprise, for example, a mammal. In some such embodiments, a mammal includes, for example and without limitation, a household pet (e.g., a dog, a cat, a rabbit, a ferret, a hamster, etc.), a livestock or farm animal (e.g., a cow, a pig, a sheep, a goat, a chicken or another poultry), a horse, a monkey, a laboratory animal (e.g., a mouse, a rat, a rabbit, etc.) and the like. Subjects can also include fish and other aquatic species. In a preferred embodiment, the subject in need thereof in accordance with technologies described herein is a human.

In some aspects, technologies disclosed herein can be practiced in any subject that has (e.g., has been diagnosed with) a brain tumor (e.g., a glioblastoma). A subject having a tumor (e.g., a brain tumor) is a subject that has detectable tumor cells.

In some aspects, technologies of the present disclose can be utilized in a subject that has brain cancer (e.g., glioblastoma). A subject that has cancer is a subject that has detectable cancer cells. In some embodiments, a cancer involves one or more tumors (e.g., brain tumors).

Tests for diagnosing brain cancers to be treated by technologies described herein are known in the art and can be readily understood and utilized to the ordinary medical practitioner. Such tests include, for example and without limitation, magnetic resonance imaging (MRI), computed tomography (CT) scan, positron emission tomography (PET) scan, diagnostic angiogram, myelogram, etc. Generally, medical practitioners also take a full medical history and conducts a complete physical examination in addition to the tests listed above.

In some aspects, technologies of the present disclosure can be utilized in a subject that has a glioblastoma (e.g., glioblastoma multiforme GBM). Methylation of the promoter region of the DNA repair enzyme gene, O⁶-methylguanine-DNA methyltransferase (MGMT), is a prognostic factor for glioblastoma. MGMT removes alkyl groups from guanine in the DNA.

Without wishing to be bound by any one theory, it is understood that MGMT removal of alkyl groups from guanine in the DNA counteracts the therapeutic efficacy of alkylating chemotherapeutics, such as temozolomide (TMZ). Epigenetic silencing (e.g., methylation) of the promoter region of MGMT might lead to transcriptional repression and a decreased MGMT protein expression and is associated with an improved response to TMZ chemotherapy and longer overall survival of GBM patients. In some embodiments, a subject that has glioblastoma comprises a methylated MGMT promoter. In some such embodiments, a subject that has glioblastoma comprises an unmethylated MG™ promoter. MGMT methylation can be assessed by a plurality of methods known in the art, including, for example, by methylation-specific PCR (MSP), high-resolution melting PCR (HRM), or pyrosequencing. A cut-off value of 8-10% can distinguish unmethylated from methylated MGMT promoters. See, e.g., Feldheim J. et al., Cancers (Basel). 2019 Nov. 21; 11(12):1837.

In some aspects, the subject in need thereof in accordance with technologies of the present disclosure has undergone one or more other cancer therapies (e.g., chemotherapy, radiotherapy, immunotherapy). In some embodiments, the subject in need thereof has previously undergone or more other cancer therapies and the subject's cancer has relapsed. In some embodiments, the subject in need thereof has previously undergone one or more other cancer therapies and the subject has developed resistance to the one or more other cancer therapies. In some embodiments, the subject in need thereof is in remission (e.g., partial remission, complete remission). In some embodiments, the subject in need thereof is refractory to one or more other cancer therapies.

In some embodiments, the subject in need thereof is an adult. In some embodiments, the subject in need thereof is a human subject over 18 years of age. In some embodiments, the subject in need thereof is a human subject over 21 years of age. In some embodiments, the subject in need thereof is a human subject over 30 years of age. In some embodiments, the subject in need thereof is a human subject over 65 years of age. In some embodiments, the subject in need thereof is a human subject under 18 years of age. In some embodiments, the subject in need thereof is a human subject under 65 years of age (or between 18 and 65 years of age, between 21 and 65 years of age, or between 30 and 65 years of age). In some embodiments, the subject in need thereof is a pediatric subject (e.g., a human subject under the age of 12).

### Combination Therapies

In some aspects, technologies of the present disclosure further include methods of treating a brain tumor (e.g., a glioblastoma) with additional agents that enhance therapeutic responses, such as, for example, enhance an anti-tumor response in a subject in need thereof and/or are cytotoxic to the tumor, such as a chemotherapeutic agents.

In some aspects, methods of treatment described herein is administered to a subject in need thereof in combination with one or more anti-cancer therapies, including, for example and without limitation, a chemotherapy, a radiation therapy, a stem cell transplant, an anti-cancer small molecule, an additional immunotherapy, or any other cancer therapy known in the art.

Suitable therapeutic agents for use in combination therapy with methods of the present disclosure include small molecule chemotherapeutic agents. Such chemotherapeutic agents include, for example and without limitation, alkylating agents, a nitrosourea agent, an antimetabolite, a platinum complex derivative, a topoisomerase inhibitor, an aromatase inhibitor, an alkaloid derived from a plant, a hormone antagonist, an antitumor antibiotic, and a P-glycoprotein inhibitor. Specific examples of chemotherapeutic drugs that can be used in the combination therapy described herein include, without limitation, taxol, paclitaxel, nab-paclitaxel, 5-fluoro uracil (5-FU), gemcitabine, doxorubicin, daunorubicin, colchicin, mitoxantrone, tamoxifen, cyclophosphamide, mechlorethamine, melphalan, chlorambucil, busulfan, uramustine, mustargen, ifosamide, bendamustine, carmustine, lomustine, semustine, fotemustine, streptozocin, thiotepa, mitomycin, diaziquone, tetrazine, altretamine, dacarbazine, mitozolomide, temozolomide, procarbazine, hexamethylmelamine, altretamine, hexalen, trofosfamide, estramustine, treosulfan, mannosulfan, triaziquone, carboquone, nimustine, ranimustine, azathioprine, sulfanilamide, fluoropyrimidine, thiopurine, thioguanine, mercaptopurine, cladribine, capecitabine, pemetrexed, fludarabine, methotrexate, hydroxyurea, nelarabine or clofarabine, cytarabine, decitabine, pralatrexate, floxuridine, thioquanine, azacitidine, cladribine, pentostatin, mercaptopurine, imatinib, dactinomycin, cerubidine, bleomycin, actinomycin, luteomycin, epirubicin, idarubicin, plicamycin, vincristin, vinblastine, vinorelbine, vindesine, vinflunine, paclitaxel, docetaxel, etoposide, teniposide, periwinkle, vinca, taxane, irinotecan, topotecan, camptothecin, teniposide, pirarubicin, novobiocin, merbarone, aclarubicin, amsacrine, antiandrogen, anti-estrogen, bicalutamide, medroxyprogesterone, fluoxymesterone, diethylstilbestrol, estrace, octreotide, megestrol, raloxifene, toremifene, fulvestrant, prednisone, flutamide, leuprolide, goserelin, aminoglutethimide, testolactone, anastrozole, letrozole, exemestane, vorozole, formestane, fadrozole, androstene, resveratrol, myosmine, catechin, apigenin eriodictyol isoliquiritigenin, mangostin, amiodarone, azithromycin, captopril, clarithromycin, cyclosporine, piperine, quercetine, quinidine, quinine, reserpine, ritonavir, tariquidar, verapamil, cisplatin, carboplatin, oxaliplatin, transplatin, nedaplatin, satraplatin, triplatin and carboplatin.

In some embodiments, methods of the present disclosure further comprise administration of radiotherapy.

In some embodiments, methods of the present disclosure further comprise administration of antibiotics.

In some embodiments, methods of the present disclosure further comprise concomitant use of Tumor Treating Fields (TT Fields).

In certain embodiments, any therapy described herein can be administered before, during (e.g., concurrently), and/or after administration of an anthracycline, an immune checkpoint blockade, and low-intensity pulsed ultrasound and microbubbles as described herein. In some embodiments, the subject in need thereof in accordance with technologies of the present disclosure has not previously received an anti-cancer therapy. In some embodiments, the subject in need thereof in accordance with technologies of the present disclosure has been previously treated with an anti-cancer therapy (e.g., a chemotherapy, a radiotherapy, a stem cell transplant).

### Kits

In one aspect, the present disclosure provides kits comprising one or more containers comprising (i) an anthracycline as described herein; (ii) an immune checkpoint modulator as described herein; (iii) an ultrasound device and compositions for administering microbubbles; and (vi) optionally, instructions for use.

A container may include, for example and without limitation, a vial, well, test tube, flask, bottle, syringe, infusion bag, or other container means. Where an additional component is included in the kit, the kit can contain additional containers into which this component may be placed. Containers and/or kits can comprise labeling with instructions for use and/or warnings.

### Examples

Glioblastoma (GBM) is the most common and most aggressive primary brain tumor in adults with a poor prognosis and a 5-year survival averaging less than 10% (1-3). Despite preclinical evidence strongly suggesting efficacy of immunotherapy, its clinical application has yielded disappointing results in unselected patient populations with GBM (4-8). Several factors may contribute to this discrepancy, including the blood-brain barrier (BBB) that limits effective drug delivery to regions of infiltrative disease within the peri-tumoral brain (9-11); the immunosuppressive tumor microenvironment is marked by inherently low tumor immunogenicity and a paucity of lymphocyte infiltration (12-15); and the inter- and intra-tumor heterogeneity that results in an inconsistent and unpredictable response to therapies including immunotherapy (16-20).

Immune checkpoint blockade (ICB) targeting cytotoxic T-lymphocyte antigen 4 (CTLA-4) is an established form of immunotherapy and has been investigated in clinical trials for GBM (NCT02311920, NCT04396860) (21-23). This form of ICB relies partly on T cell priming (24) and is also thought to promote the depletion of intra-tumoral immunosuppressive regulatory T cells (Tregs), however this remains controversial in the clinic (25). Anti-CTLA-4 antibodies (aCTLA-4) bound to Tregs, can co-engage, activating Fcγ receptors (FcγRs) expressed on macrophages or nature killer (NK) cells to promote antibody-dependent cellular phagocytosis (ADCP) or cytotoxicity (ADCC) respectively (24, 26-28). Notably, depletion of intra-tumoral Tregs and therapeutic outcomes can be significantly enhanced by Fc-engineering techniques that improve binding to FcγRIIA and/or FcγRIIIA (29). Further recent studies have demonstrated that the ability of aCTLA-4 to co-engage FcγRIIIA on antigen-presenting cells (APCs) such as dendritic cells or macrophages are critical for promoting T cell priming and activation (24) and drive myeloid activation and type I interferon signaling (30), key mechanisms that are thought to be important for treating poorly immunogenic and ‘cold’ tumors. The importance of co-engaging FcγRIIIA with aCTLA-4 have also been shown clinically (29).

In metastatic melanoma, patients with inflamed tumors treated with ipilimumab, an IgG1 aCTLA-4, showed better overall survival if they expressed the high affinity variant of FcγRIIIA (158-V/V or 158-V/F) compared to those who only expressed the low affinity variant (158-F/F) consistent with the predicted binding affinity of IgG1 antibodies (31). Together, these studies suggest that conventional aCTLA-4 antibodies may have limited therapeutic activity due to suboptimal co-engagement of activating FcγRs.

To address this limitation, a second-generation aCTLA-4 ICB antibody, botensilimab, that is engineered in the Fc region with point mutation (S239D/A330L/I332E; DLE) that exhibits a higher affinity for FcγRIIIA and increased effector functions compared to wild-type Fc has been developed (24, 32). In phase 1 clinical trials, botensilimab monotherapy or in combination with balistilimab (aPD-1) demonstrated unprecedented activity in immune-resistant ‘cold’ tumors such as micro-satellite stable colon cancer, and in other heavily pre-treated patients who failed prior immunotherapy.

Applicant described the investigation of a mouse surrogate of botensilimab, referred to as FcE-aCTLA-4, in preclinical immune-resistant GBM models. While the FcE-aCTLA-4 therapy shows superior monotherapy efficacy compared to conventional aCTLA-4, combination therapy will be essential to overcome tumor heterogeneity and enhance responses in ‘cold’ and poorly immunogenic tumors (35). Applicant therefore investigated the use of FcE-aCTLA-4 with doxorubicin (DOX), as this anthracycline has been reported to exert an immunomodulatory effect facilitating type I interferon response by cancer cells (36-38). A recent clinical trial demonstrated that induction therapy with DOX potentiated the response to PD-1 blockade in patients with breast cancer, doubling the objective response rate (38). Therefore, Applicant hypothesized that the combination of DOX and anti-PD-1 (aPD-1) could modulate the tumor immune-microenvironment in GBM and further improve the response to FcE-aCTLA-4.

The BBB is a major impediment to successful drug therapy in the brain. Whereas the core of GBM might exhibit BBB breakdown and permeability of systemic drugs, the enhancing tumor core is commonly resected as part of the standard of care. Yet, GBM recurrence is virtually always driven by infiltrating tumor cells residing in the peri-tumoral brain where the BBB is intact and penetration of systemic agents is poor. To address the limited penetration of systemically delivered ICB antibodies and DOX across the BBB, Applicant investigated the use of low-intensity pulsed ultrasound in combination with microbubbles (LIPU/MB). This innovative technique temporarily opens the BBB leading to a significant increase in the concentration of systemically administered drugs in the brain of GBM patients (37, 39-42).

Here, Applicant characterized the expression of CTLA-4 and FcγRs in murine and human glioma tumor microenvironment and assessed the immune modulating impact and therapeutic potential of an FcE-aCTLA-4 compared to conventional aCTLA-4 therapy in mouse models of glioma. FcE-aCTLA-4 exhibited high affinity binding to human FcγRIIIA and to the mouse ortholog FcγRIV, that Applicant found to be highly expressed by tumor-associated macrophages/microglia (TAM) in mouse and human GBM. In in vitro studies, FcE-aCTLA-4 exhibited superior antibody-dependent Treg-specific phagocytosis by TAM compared to conventional aCTLA-4, and similarly in vivo, this agent exhibited superior Treg depletion in intracranial murine gliomas. Notably, FcE-aCTLA-4 therapy demonstrated superior efficacy than conventional aCTLA-4, both as monotherapy and in combination with aPD-1 and DOX in immunotherapy-resistant murine gliomas. Applicant also reported an analysis of four GBM patients treated with LIPU/MB-based delivery of aPD-1 and DOX. Applicant demonstrated an upregulation of FcγRIIIA on TAMs that Applicant attributed to DOX exposure. The combination of FcE-aCTLA-4, aPD-1 and DOX delivered with LIPU/MB achieved a >90% cure rate in multiple immunotherapy-resistant murine glioma models. Applicant's findings provide a novel immunotherapeutic strategy and combined treatment strategy for GBM, to be investigated in an upcoming clinical trial.

### Example 1: Materials and Methods

The following materials and methods were used for the following Examples.

Study design: This a translation study that sought to determine the ability of the US technology to improve the penetration of DOX and anti-PD-1 for gliomas. Murine glioma models and GBM patient tumor samples treated with liposomal DOX and anti-PD-1 delivered with US/MB were analyzed to evaluate immunological responses by T cells and glioma-associated microglia and macrophages. Efficacy of the combinatorial treatment was evaluated in glioma-bearing mice.

Ultrasound-mediated BBB opening in GBM patients and treatment with liposomal DOX and pembrolizumab. As part of a phase I clinical trial evaluating nab-paclitaxel (NCT04528680), the SonoCould-9 device (CarThera, Paris, France) was implanted during neurosurgery targeting the tumor and peritumoral brain in recurrent GBM patients. Patients received additional treatment cycles with sonications in an outpatient setting in which the US device was activated by percutaneous access with a single-use transdermal needle concomitantly with the injection of 10 μL/kg of IV microbubbles (DEFINITY, Lantheus, Billerica, USA) and 200 mg of pembrolizumab and 30 mg of liposomal DOX. Steroids and mannitol were avoided to perform pharmacokinetic studies. 48 hours after the treatment with liposomal DOX and pembrolizumab GBM patients underwent surgery for tumor resection. The acquisition of non-eloquent peritumoral brain sample either sonicated or not was performed when justified as per standard neurosurgical technique as reported previously (Gill, B. J. et al., Proc Natl Acad Sci USA 111, 12550-12555 (2014); Chang, P. D. et al., AJNR Am J Neuroradiol 38, 890-898 (2017)). Institutional review board (TRB) approval was obtained from our institution. The study was conducted in accordance with the institutional ethical regulations and the Declaration of Helsinki. Informed consent was obtained from all GBM patients.

Determination of the DOX concentration in blood, tumor and peritumoral brain tissue. Two GBM patients undergoing surgery for resection of tumor with implantation of the Sonocloud-9 US device were studied for this analysis. Brain parenchyma that did not display enhancement in contrast MRI was considered peritumoral brain. Sonicated and non-sonicated peritumoral brain samples were identified taking into account their location relative to the sonication field of the US device. Non-sonicated peritumoral brain samples were acquired first with the use of a new set of surgical instruments for each sample. Every peritumoral brain tissue was washed with saline solution to remove blood. These samples were sectioned into 30 mg pieces and were flash-frozen for further quantification of DOX levels. DOX was quantified in tumor, peritumoral, and plasma using LC/MS (6500 QTRAP AB Sciex equipped with a SIL-20AC XR HPLC, Shimadzu Scientific Instruments).

Ultrasound-mediated BBB opening in mouse models. All sonication procedures were performed as previously described in Zhang, D. Y., et al., Clin Cancer Res 26, 477-486 (2020). A preclinical US device (Sonocloud Technology) manufactured by CarThera (France) along with IV injection of 100 μL of MB (Lumason, Bracco) reconstituted following manufacturer's protocol. C57BL/6 mice were employed for sonication experiments in which they were anesthetized with ketamine/xylazine cocktail intraperitoneally (ketamine 100 mg/kg, xylazine 10 mg/kg). Hair from the mice heads was removed using Nair hair removal lotion followed by washes with warm water. Different doses of liposomal DOX, mouse PD-1 blocking antibody, isotype IgG4 antibody, nivolumab and/or fluorescein were injected via retro-orbital route following by MB injection and sonication. For US-mediated BBB opening, mice were placed in a supine position with their heads in a 10 mm diameter flat US transducer holder touching the degassed water contained in the US device. The sonication procedure was performed for 60 seconds using a 25,000-cycle burst at a 1 Hz pulse repetition frequency and an acoustic pressure of 0.3 MPa predefined in the US device. Mice were removed from the US transducer holder and put in a clean cage placed upon a heating pad and monitored until they recovered from anesthesia.

Flow cytometry of PDX cell lines and HMC3 cells. PDX cell lines were kindly gifted. HMC3 cells were acquired from ATCC. GBM6, GBM63 and HMC3 cells were seeded on 6-well plates for treatment with increasing concentrations of DOX hydrochloride (D1515, Sigma Aldrich), IFN-γ (300-02, Peprotech) or 50 μM of TMZ (Accord). PDX and HMC3 cells were treated for 5 hours followed by 3 washes with PBS and cultured afterwards with Dulbecco's modified Eagle's medium (Corning) supplemented with 10% FBS (Hyclone) and 1% penicillin/streptomycin (Corning). 72 hours after treatment, PDX cells were harvested using Accutase cell detachment solution (Corning) and were stained initially with eBioscience Fixable Viability Dye eFluor 780 (Thermo Fisher) at 1:1000 dilution. Staining with HLA ABC AF-647 (311414) and HLA-DR BV421 (307636) was done for PDX cells. IFN-γ AF-700 (502520), HLA ABC FITC (311404), PD-L1 Pe-Cy7 (374506). All antibodies for this experiment were from Biolegend. Data was acquired using BD FACSymphony Flow Cytometer.

Multiplex immunofluorescence of human GBM samples. 5 m sections were cut from FFPE GBM samples. Slides were loaded onto Leica Bond Rx where they were baked at 60° C. for 30 min followed by a dewaxing process consisting in rinsing the slides three times with 150 μL of preheated Bond dewax solution and three rinses of 150 μL ethanol. Bond ER1 solution was used for antigen retrieval for antibodies incubated with pH6. Bond ER2 solution was used for antigen retrieval for antibodies incubated with pH9. Slides were incubated at 99° C. for 20 minutes. For the next steps, the Opal 7-color IHC kit (NEL821001KT, Akoya Biosciences) was employed. Primary antibodies were diluted using the Opal Antibody Diluent/Block solution provided with the kit. The following antibodies were used: SOX2 (Abcam, clone EPR3131, 1:5000, pH9) paired with Opal 620, TMEM119 (Sigma-Aldrich, 1:250, pH6) paired with Opal 520, CD163 (Abcam, clone EPR19518, 1:600, pH9) paired with Opal 650, IFNγ (Abcam, clone IFNG/466, 1 μg/mL, pH9) paired with Opal 570, HLA-DR (Abcam, clone TAL 1B5, 1:1000, pH6) paired with Opal 570, HLA-ABC (Abcam, clone EMR8-5, 0.3 μg/mL, pH6) paired with Opal 570. Multiplex staining was performed with an antigen retrieval step, protein blocking, epitope labeling and signal amplification between each cycle. At the end of all cycles, Spectral DAPI (Akoya Biosciences) was used to counterstain the slides, which were mounted with long-lasting aqueous-based mounting medium.

Imaging and analysis of multiplex immunofluorescence images. Images were acquired using the Vectra 3 Automated Quantitative Pathology Imaging System from Akoya Biosciences. Multispectral images (MSI) were acquired in tumor regions previously delineated by a neuropathologist. Spectral unmixing was performed for all the MSI files using a spectral library for all Opal dyes as a reference in inForm Tissue Finder software 2.6. (Akoya Biosciences). Cell segmentation was performed employing DAPI to delineate nuclei as well as phenotyping of particular cell types including SOX2THLA ABC⁺, SOX2⁺ HLA DR⁺, TMEM119⁺ IFN-7⁺, TMEM119⁺ HLA ABC⁺, and CD163 HLA ABC⁺. Next, processed images from all tumor samples were exported to data tables. These exported files were further processed in R employing the R packages Phenoptr and PhenoptrReports to merge and create consolidated files for each tumor sample. Consolidated files were used to quantify the phenotypes of interest. UMAP plots for SOX2⁺, HLA ABC⁺, HLA DR⁺ were created from multiplex immunofluorescence data. R v. 4.0.3 and RStudio were used to install and run the following packages: flowCore, shiny, reticulate, and cytofkit2 (see, Chen, H., et al., PLoS Comput Biol 12, e1005112 (2016)). Cells that were negative for all phenotypes were selected out in all data tables. Next, filtered .txt files were loaded to inForm2fcs to create fcs files. The data from pre-treatment and on-treatment GBM samples from 2 patients were loaded to cytofkit2 to cluster all cells in one UMAP plot. For the clustering process, Ceil was the merged method employed along with cytofAsinh as a transformation method with a fixed number of 10,000 cells. SOX2 and CD163 were used to perform supervised clustering analysis. UMAP was the dimensionality reduction method employed. The new generated .Rdata file was uploaded to cytofkit2 to visualize the data. In the marker panel, expression level plots were visualized using the spectral2 color palette centered for SOX2, HLA ABC, and HLA DR. To evaluate IFN-γ by glioma-associated myeloid cells, CD163⁺ and TMEM119⁺ cells were isolated in R from pre-treatment and on-treatment GBM samples. Next, fcs files containing only myeloid cells were created using inForm2fcs using .txt files. Newly created fcs files were uploaded to Matlab v. R2021b. Data were transformed (asinh) using cofactor 5 and plotted in a PCA plot considering CD163 and TMEM119 expression, IFN-γ was then assessed in myeloid cells from pre-treatment and on-treatment GBM samples.

Processing and flow cytometry analysis of human GBM-infiltrating T cells. Non-treated and DOX-treated GBM samples were acquired by the Nervous System Tumor Bank at Northwestern University. Tumor samples were immediately processed into single-cell suspension using the Adult Brain Dissociation Kit (130-107-677, Miltenyi Biotec) following manufacturer's protocol. Single-cell suspension of GBM samples was cryopreserved using RPMI media (Corning), DMSO, and FBS (Hyclone) for further staining with conjugated antibodies. Cryopreserved non-treated and DOX-treated single-cell suspensions were thawed at the same time for stimulation and staining. Cells were washed with complete RPMI media and re-stimulated with cell activation cocktail (423303, Biolegend) for 4 hours. After 4 hours re-stimulation, cells were washed with 1×PBS and stained with Zombie-NIR (Biolegend 423105) for cell viability. Following viability staining, cells were stained for surface markers with the following fluorescently conjugated antibodies: CD45 BV605 (368524, Biolegend), CD3 PerCP (300326, Biolegend), CD8 PE-Cy7 (300914, Biolegend), CD4 FITC (317408, Biolegend). Cells were then fixed with Fixation/Permeabilization concentrate (00-5123-43, eBiosciences) and stained intracellularly with IFN-γ Alexa Fluor 700 (505824, Biolegend).

Data was acquired using BD FACSymphony Flow Cytometer and analyzed using FlowJo (BD). To visualize the expression of IFN-7, and PD-1 by T cells in scatter plots, the associated Matlab based tool, cyt3 (Amir, e.-A., et al., Nat Biotechnol 31, 545-552 (2013), was employed. Fcs files including CD3⁺ live single cells were uploaded into cyt3 for analysis. Subsampling was performed to represent 5000 cells for each group: No treatment and liposomal DOX+pembrolizumab+US/MB. Expression of each marker was normalized equally across the board.

Cell lines and implantable syngeneic murine glioma models for survival and immunophenotyping studies. Murine GL261 cell line was acquired from the National Institutes of Health. CT2A cell line was acquired from Millipore. GL261 and CT2A were cultured in Dulbecco's modified Eagle's medium (Corning) supplemented with 10% FBS (Hyclone) and 1% penicillin/streptomycin (Corning) at 37° C. in incubators with humified atmosphere of 5% CO₂ and 95% air. All murine cell lines used for this study were routinely tested for mycoplasma and were confirmed negative before intracranial orthotopic injection.

To perform intracranial injection of syngeneic murine glioma cell lines, mice were anesthetized with ketamine/xylazine cocktail. Artificial tears were used to prevent eye drying and protect the eye. The surgical site was cleaned with a swab of povidone-iodine and 70% ethanol. An incision was done along the sagittal axis of the mouse head to expose the skull underneath. Then, using a sterile handheld drill (Harvard Apparatus) a burr hole was created 3 mm lateral and 2 mm caudal relative to sagittal and bregma sutures. after which the mice were placed in a stereotaxic device (Harvard Apparatus). 200,000 GL261 or 100,000 CT2A glioma cells were injected into the left hemisphere of brain at 3 mm depth through the burr hole. After injection of glioma cells, the incision was closed using 9 mm stainless steel wound clips. On day 7 and 14 after IC implantation of glioma cell lines, mice were treated with liposomal DOX obtained from the pharmacy of Northwestern Memorial Hospital. On day 7, 10, 14, and 17 after IC injection of glioma cells, mice were treated with either 200 g of anti-mouse PD-1 (CD279) BE0146 (BioXcell) or rat IgG2a isotype control BE0089 (BioXcell).

Immunophenotyping and treatment of glioma-bearing mice with increasing doses of liposomal doxorubicin. Tumor and blood samples were processed for immunophenotyping analysis as previously described (Lee-Chang, C., et al., Cancer Immunol Res 7, 1928-1943 (2019)). All mouse antibodies were acquired from Biolegend. Dead cells and debris were removed from the analysis employing the eBioscience Fixable Viability Dye eFluor780 (Thermo Fisher). Data was acquired using BD FACSymphony Flow Cytometer and all analysis employing flow cytometry data used FlowJo v. 10.7.1. To create tSNE plots using flow cytometry data, the gating comprising single live cells for each group of samples was used and exported with all compensated parameters. Similar to the multiplex immunofluorescence data, cytofkit2 (Chen, H., et al., PLoS Comput Biol 12, e1005112 (2016)) was used for visualization. The new generated FCS files containing the single live cell data were uploaded for further data processing in the cytofkit2 app. Ceil was the merged method employed along with autoLgcl as a transformation method with a fixed number of 2500 cells. The CD11b, CD4, and CD8 markers were used to perform supervised clustering analysis. tSNE was the dimensionality reduction method employed with a tSNE perplexity of 30, tSNE Max Iterations of 1000, and seed 42. Rphenograph was used as the clustering method. Files and results were obtained using the previous parameters. Next, the new generated .Rdata file was uploaded to cytofkit2 to explore the data. In the marker panel, expression level plots were visualized using the spectral2 color palette, local scaling range, and centered for CD4, CD8, CD11b, CD45, and IFNγ.

IFNγ ELISpot in PBMCs from mice treated with liposomal DOX. GL261 glioma cells were injected intracranially in C57BL/6 mice. Mice were treated on day 7 and day 14 with liposomal DOX 5 mg/kg through retroorbital route. On day 19, mice were anesthetized with ketamine/xylazine cocktail intraperitoneally (ketamine 100 mg/kg, xylazine 10 mg/kg). Extraction of blood was done by introducing a 1 ml TB syringe with a 25 G needle into the heart followed by slowly pulling of the blood to the syringe. Next, the extracted blood was transferred to a K2 EDTA (K2E) blood collection tube (BD vacutainer) and placed in a shaker for 15 min at room temperature. Blood was centrifuged for 30 minutes to isolate plasma and cells. Cells were counted to get 100,000 PBMCs for further steps. Mouse IFN-γ Single-Color ELISpot (ImmunoSpot) was used to detect the number of IFNγ spots in PBMCs of GL261-bearing mice. Following isolation of PBMCs, these cells were incubated with GL261 tumor lysate. ELISpot assay was performed following manufacturer's protocol. Data and images of the IFNγ spots in 96-well plates were acquired using the classic AID EliSpot reader (Autoimmun diagnostika GBMH).

Determination of the concentration of nivolumab in brain from C57BL/6 mice. Non-tumor bearing C57BL/6 mice were used for this experiment. Nivolumab (Brystol Myers Squibb) was acquired from the pharmacy of Northwestern Memorial Hospital. Nivolumab ELISA kit (ab237651, Abcam) was employed to determine nivolumab concentrations in the brain and blood. Mice were anesthetized with ketamine/xylazine cocktail and treated with artificial tears once fully anesthetized. Next, mice were treated with nivolumab through retro-orbital route. The dose used for mice was calculated based on allometric scaling which considers the animal equivalent dose based on body surface area converted from the human dose of 3 mg/kg of nivolumab (Nair, A. B. et al., J Basic Clin Pharm 7, 27-31 (2016)). After treatment with nivolumab, mice were placed in the US transducer holder for sonication as described above. Intravenous NaFI (Sigma-Aldrich) previously dissolved in PBS was administered immediately after sonication at a dose of 20 mg/kg in 100 μL. 1 and 4 hours after sonication, mice were euthanized using a CO₂ chamber and brains were harvested and placed in PBS. Mouse brains were imaged using the Nikon AZ100 epifluorescent microscope. With a clean scalpel, fluorescent areas of the brains representing regions of BBB disruption were dissected, separated, and weighed. Fluorescent brain regions were homogenized in 1 mL of Assay buffer (included in the Nivolumab ELISA kit) using a tissue grinder with PTFE pestle (Kimble, Capitol Scientific). Brain samples were diluted in 1:10 ratio and blood samples were diluted in a 1:100 ratio using the Assay buffer. The rest of the procedure was done following manufacturer's kit protocol. Absorbance of samples (set to 450 nm) was read in Cytation 5 multi-mode reader (Biotek). A standard curve was generated employing the standards included in the kit to determine the concentration of nivolumab in each sample by interpolating values of the standard curve.

Determination of the concentration of pembrolizumab in human glioma and peritumoral brain tissue Two GBM patients undergoing surgery for resection of tumor with implantation of the Sonocloud-9 US device were studied for this analysis. Pembrolizumab ELISA kit (ab237652, Abcam) was employed to determine pembrolizumab concentrations. After surgical resection, all samples were weighed. Samples were homogenized in 1 mL of Assay buffer (included in the Pembrolizumab ELISA kit) using a tissue grinder with PTFE pestle (Kimble, Capitol Scientific). Brain samples were not further diluted, and blood samples were diluted in a 1:100 ratio. The rest of the experiment was done following manufacturer's kit protocol. Absorbance of samples (set to 450 nm) was read in Cytation 5 multi-mode reader (Biotek). A standard curve was generated employing the standards included in the kit to determine the concentration of pembrolizumab in each sample.

Analysis of SoniMel tumor volume of a brain metastatic patient. This patient was part of an ongoing clinical trial (NCT04021420) evaluating the combination of the immune checkpoint antibodies, ipilimumab and nivolumab, with BBB disruption employing the SC1 US device. The zone targeted by the skull implanted SC1 US emitter was modeled with a cylindrical region of interest (ROI) with a 10-mm diameter and 55-mm length. This ROI covered a part of the left-hemisphere tumoral mass and a part of its margin. The patient received administration every three weeks of 1 mg/kg of nivolumab (Brystol Myers Squibb) during 30 minutes of infusion and 3 mg/kg of ipilimumab (Brystol Myers Squibb) during 90 minutes of infusion prior to sonication. The SC1 US device was activated in combination with IV injection of microbubbles (0.1 mL/kg, SonoVue, Bracco) for a duration of 270 seconds at an acoustic pressure of 0.78 MPa, similar to the parameters used in a recent clinical trial in patients with recurrent GBM (Idbaih, A., et al., Clin Cancer Res 25, 3793-3801 (2019)). The evolution of the tumor volumes was evaluated for both sonicated and non-sonicated tumor masses using a semiautomatic active contour segmentation method (ITK Snap) (Yushkevich, P. A., et al., Neuroimage 31, 1116-1128 (2006)) using contrast-enhanced gadolinium T1w MR images with a threshold set at 1.25 times the median brain intensity.

Animal studies All animal experiments were performed in accordance with and approved by Northwestern University's Institutional Animal Care and Usage Committee under the protocol no. IS000017464. Six- to twelve-week-old male and female C57BL/6 mice were purchased from Charles River Laboratories for these experiments. All animals were housed in a pathogen-free animal facility at Northwestern University. The breeders of the Cd8^{-/-} mice were purchased from the Jackson Laboratory (B6.129S2-Cd8a^{tm1Mak}/J, stock #002665. The genotyping protocol was performed following the recommendation of the Jackson Laboratory and separated by gel electrophoresis on a 1.5% agarose gel.

Statistical analysis: Data are shown as mean±SEM or mean±s.d. as indicated in each figure legend. Data following normal distributions were subjected to unpaired and paired Student's t test. One-way ANOVA was used to compare means between groups and to determine which specific groups different from each other Dunnett's or Tukey's multiple comparison test were employed. The lme4⁷⁶ was used to perform a linear mixed effects analysis of the relationship between DOX concentrations in peritumoral regions and sonication. As fixed effects, sonication was entered into the model. As a random effect, patients were had as an intercept. P values were obtained by likelihood ratio tests of the full model with the effect in question against the model without the effect in question. Kaplan-Meier curves were used to plot survival results analyzed using log-rank test. α=0.05 was used to determine statistical significance. After intracranial injection of murine glioma cells, tumor-bearing mice were randomized to each treatment group. For each experiment, the replicate numbers are reported in each figure legend. Prism v. 9 (GraphPad, San Diego, CA, USA), Matlab v. R2021b (9.11.0), R v. 4.0.3 and RStudio were used for statistical analysis and generation of figures.

Cell culture. The mouse GBM cell line GL261 was obtained from the National Institutes of Health (NIH) and the line CT-2A was purchased from Millipore. Both cell lines were cultured in Dulbecco's modified Eagle's medium (Gibco) supplemented with 10% fetal bovine serum (Gibco), and 1% penicillin/streptomycin (Gibco) in 5% CO₂ incubators at 37° C. QPP4 cells derived from Nestin-CreER^{T2} Qk^{L/L}; Trp53^{L/L}; Pten^{L/L} mice (QPP) were provided from Dr. Amy B. Heimberger (Northwestern University) (49, 69). QPP4 cells were maintained in Dulbecco's modified Eagle's medium/F-12 (Gibco) supplemented with B-27 (Gibco), recombinant epidermal growth factor (EGF, 20 ng/ml, PeproTech), and recombinant basic fibroblast growth factor (bFGF, 20 ng/ml, PeproTech).

Animal Experiments. All mouse protocols performed in this study were approved by Northwestern University Institutional Animal Care and Use Committee (IACUC) under the approval number IS00017189. Six- to eight-week-old wildtype C57BL/6 mice were purchased from Charles River Laboratories. All animals were housed in a pathogen-free animal facility at Center for Comparative Medicine, Northwestern University. All murine cell lines used for this study were routinely tested for mycoplasma and were confirmed negative before intracranial orthotopic injection. To perform intracranial injection of syngeneic murine glioma cell lines, mice were anesthetized with ketamine/xylazine cocktail. Artificial tears were used to prevent eye drying and protect the eye. The surgical site was cleaned with a swab of povidone-iodine and 70% ethanol. An incision was done along the sagittal axis of the mouse head to expose the skull underneath. Then, using a sterile handheld drill (Harvard Apparatus) a burr hole was created 3 mm lateral and 2 mm caudal relative to the bregma with mice placed on a stereotaxic device (Harvard Apparatus). 100,000 GL261, QPP4 or 75,000 CT-2A glioma cells were injected into the left hemisphere of brain at 3 mm depth through the burr hole. Mice were monitored daily and were euthanized when they approached the endpoint (weight loss is >20% of pre-treatment body weight or loss of mobility or severe neurological disabilities such as seizure, circular motion, etc.) as described in the IACUC protocol.

Antibodies and mouse Fc-FcγR cellular binding. Fc-engineered mouse reactive anti-CTLA-4 antibody were provided by Agenus Inc. (Lexington, Massachusetts). The sequences of the variable regions of the heavy and light chains of anti-mouse CTLA-4, clone 9D9 (RRID: AB_10949609) was used to generate surrogate antibodies with the constant regions of mouse IgG2b (mIgG2b) and mutated mIgG2b with amino acid substitutions S239D/A330L/I332E (mIgG2b.DLE; Fc-enhanced). Antibodies were produced using recombinant deoxyribonucleic acid (DNA) technology in a Chinese hamster ovary (CHO) mammalian cell expression system. InVivoPlus grade isotype control (clone MPC-11, mIgG2b) and mouse reactive anti-PD-1 (RMP1-14; Rat IgG2a, x) were obtained from BioXcell (USA).

CHO cells genetically engineered to express mouse FcγRI, FcγRIIB, FcγRIII, and FcγRIV were obtained from Collection de Cultures de Microorganismes, Institut Pasteur (CNCM) (70). The mouse FcγR-expressing CHO cells were incubated with titrated concentrations of anti-CTLA-4 antibodies or an isotype negative control antibody (mIgG2b). Following a one-hour incubation, binding of the antibodies was detected by flow cytometry (LSRFortessa, BD) using a PE-conjugated donkey anti-mouse IgG Fcγ fragment specific secondary antibody (Jackson ImmunoResearch).

SPR analysis. Affinities of anti-mouse CTLA-4 antibodies to mouse FcγRIIB and FcγRIV proteins, and anti-human CTLA-4 antibodies to human FcγRIIB, FcγRIIIA F158 and FcγRIIIA V158 proteins were determined by SPR using a Biacore T200 instrument (Cytiva) and a CM5 Biacore sensor chip immobilized with an anti-His antibody (Invitrogen). The different his-tagged receptors were captured on the chip and the antibodies were injected at various range of concentrations at a flow rate of 30 l/minute and a temperature of 25° C. For binding to mouse FcγRIV, antibodies were injected at concentrations ranging from 0.93 to 120 nM. For binding to mouse FcγRIIB, antibodies were injected at concentrations ranging from 62 nM to 8 μM. For binding to human FcγRIIIA F158 and V158, antibodies were injected at concentrations ranging from 0.93 to 250 nM. For binding to human FcγRIIB, antibodies were injected at concentrations ranging from 62 nM to 8 μM. The chip surface was regenerated after each cycle with 10 mM Glycine pH 2.1. Binding kinetic analyses were carried out using Biacore evaluation software (GE Healthcare version 3.0)

Immunophenotype analysis. Tumor-bearing brains and spleens from the same mice were processed for immunophenotype analysis on day 14 and 21 after intra-cranial tumor injection. Splenocytes or Percoll gradient enriched cells from brain were filtered with 70 μm cell strainer and incubated with mouse Fc block (Biolegend). Cells then underwent surface staining with primary antibodies and live/dead staining with Fixable Viability Dye eFluor 780 (eBioscience). After fixation and permeabilization (Foxp3/Transcription Factor Staining Buffer Set, eBioscience), intracellular staining was performed. Anti-mouse antibodies for analysis were BV510 anti-CD45 (Biolegend), BV605 anti-CD8a (Biolegend), PE-Cy7 anti-CD4 (Biolegend), BUV395 anti-CD11b (Biolegend), Alexa Fluor 488 anti-Foxp3 (Biolegend), PerCp-Cy5.5 anti-CTLA-4 (Biolegend), BV421 anti-IFN-γ (Biolegend), BV711 anti-CD44, Alexa Fluor 647 anti-CD19 (Biolegend), Alexa Fluor 700 anti-CD62L (Biolegend), PE anti-PD-1 (Biolegend). Flow cytometry data was acquired by the BD Symphony and analyzed by FlowJo 10.8.1 (BD).

CD4⁺ T-cell isolation and generation/TAMgeneration. Splenocytes were prepared in single-cell suspensions from C57BL/6-Foxp3-GFP mice. Purified T cells were isolated by immunomagnetic negative selection using EasySep Mouse T cell Isolation Kit (STEMCELL), and the desired cells were labeled with 1:100 PE anti-mouse CD4 antibody (BioLegend). Isolated T cells were sorted by CD4-PE and Foxp3⁺ GFP expression using a BD FACS Aria II cell sorter (BD), and the cells were activated and expanded with 2000 U/ml recombinant TL-2 (Peprotech) and Gibco Dynabeads Mouse T-Activator CD3/CD28 (Invitrogen) for three days.

Bone marrow progenitor cells were obtained from mice's tibias and femurs by removing the epiphyses of the bones and perfusing them with complete RPMI (RPMI-1640 (Corning) with L-glutamine, 10% fetal bovine serum (Gibco), 1% penicillin/streptomycin (Gibco), 1% HEPES (Gibco), 1% nonessential amino acids (Gibco), 1% sodium pyruvate (Gibco), and 0.1% 2-mercaptoethanol (Gibco)) using a 20 ml syringe and 25-gauge needle. After centrifugation, add ACK lysing buffer to incubate for 3 minutes at room temperature, wash the cells with complete RPMI, filter with 70 μm cell strainer, count, and seed with the density of 2.5×10⁵ cells/ml plus 40 μg/ml recombinant murine M-CSF in complete RPMI into 24-well plates. After three days of culture at 37° C. in a 5% CO₂ incubator, the culture medium was replaced with 50% complete RPMI and 50% conditioned medium (collected from CT-2A glioma cell culture medium) plus 40 μg/ml recombinant murine M-CSF. Cells were cultured for additional three days and collected for studies.

Multiplex immunofluorescence staining/cellular identification and phenotyping. The multiplex panel included the following unconjugated antibodies: GFAP (glioma cells), CD11c (antigen presenting cells), CD68 (pan monocyte/macrophage), CD163 (macrophage scavenger receptor), FcγRIIIA (Receptor binding the Fc region of IgG, CD16A). All antibodies were validated using conventional immunofluorescence (IF) staining in conjunction with the corresponding fluorophore and the spectral 4′,6-diamidino-2-pheynlindole (DAPI; ThermoFisher Scientific) counterstain. For optimal concentration and best signal/noise ratio all antibodies were tested at three different dilutions, starting with the manufacturer-recommended dilution (MRD), then MRD/2 and MRD/4. Secondary Alexa fluorophore 555 (ThermoFisher Scientific) and Alexa fluorophore 647 (ThermoFisher Scientific) were used at 1/200 and 1/400 dilutions respectively. The optimizations and full runs of the multiplex panel are executed using the sequential IF methodology integrated in the Lunaphore COMET™ platform (characterization 2 and 3 protocols, and sequential IF protocols, respectively). The staining was performed following automated cycles of 2 antibodies' staining at a time, followed by imaging, and elution. Images stacking and visualization was accessed immediately after concluding the staining procedure.

Slides are automatically scanned using the Lunaphore COMET™ with fluorescent high-power field scan (20× magnification microscope). The microscope captures the fluorescent signals (DAPI, TRITC and Cy5) separately at the corresponding fluorophore wavelength, with preset exposure times, and then these captures are stacked in one image (OME.TIFF) without disrupting the unique fluorescent spectral signature of the markers. The OME.TIFF images ware analyzed in Visiopharm software for immune populations' configuration and quantification.

All digitized images were analyzed using the Visiopharm software platform (Hørsholm, Denmark). A series of custom algorithms were developed for exclusion of red blood cells, nuclei detection and phenotyping individual cells. For identifying and excluding regions of excessive bleeding, Applicant trained a deep learning classifier (Deep Lab v3+ architecture; Input size=512×512 pixels; Mini-batch size=2; Learning Rate=1×10⁻⁵; 2500 iterations) using DAPI, FITC and Cy5 autofluorescence channels to automatically identify these areas and exclude them from further analysis. Training of the convolutional neural network continued until the error rate converged to less than 2%. Magnification used for this task was 0.5×. For detecting nuclei, a pre-trained deep learning algorithm available with the Visiopharm platform (U-Net architecture) was used. The convolutional neural network was trained to identify three components of the fluorescent images: 1) DAPI positive nuclei; 2) boundaries of DAPI positive nuclei; and 3) background. The algorithm magnification was set to 20× to maximize the ability to capture details in the images. Once nuclei in the sample were identified, the nuclear labels were expanded by 3 m in all directions to approximate the boundaries of cells, not just DAPI nuclei. Finally, object labels with an area of less than 11 square microns (corresponding to a radius of approximately 2 m) were removed from further analysis. The cell segmentation was confirmed via visual inspection conducted by trained personnel.

For phenotyping cells, a targeted approach to generate the specific list of biomarker combinations was used. Specifically, Applicant was interested in finding different phenotypes of myeloid cells that were positive for a single biomarker: (e.g., CD11c⁺, CD68⁺, or CD163⁺), double positive for two biomarkers (e.g., CD11c⁺CD68⁺, CD11c⁺CD163′), and triple positive (e.g., CD11c⁺CD68⁺CD163⁺) etc., and whether the myeloid cell was expressing the FcγRIIIA (i.e., FcγRIIIA⁺ or FcγRIIIA⁻). For a given cell, the classification of each biomarker was gated using two independently controlled parameters: signal intensity and percent coverage. During the design of the generalized classification algorithm, classification parameters were iteratively adjusted to maximize accuracy and minimize the occurrence of false positives and false negatives for each biomarker. Biomarker classifications were visually inspected and confirmed by multiple researchers. Once the parameters for accurate classification were optimized, those settings were applied to all images. Once the algorithms were applied to the images, a list of output variables including counts of each identified phenotype, their density, and the spatial location in Cartesian coordinates (e.g., Center X and Center Y coordinates) for each cell on the whole slide image were generated.

Single cell RNA seq in human patient samples and mouse CT-2A glioblastoma. From the publicly available database hosted on the Broad Institute Single Cell Portal, the single-cell RNA sequencing dataset corresponding to 201,986 single cells from 44 glioma tumor fragments (44) was used to perform downstream analysis. Cell Ranger V5 generated filtered feature counts, and single cells defined as low-quality (<500 expressed genes, >20% mitochondrial transcripts or >50% ribosomal transcripts) were removed. Following the removal of low-quality cells, Seurat V4.1.1 was used to normalize and cluster the single cells after which gene expression counts were normalized to the library size and log 2-transformed. Then, principal component analysis was applied to reduce the dimensionality of the dataset. Seurat V4.1.1 identified cluster-specific marker genes and allowed for the visualization of cell types. The R package SingleR V1.10.0 was then used to identify sub-cell types in an unbiased, reference transcriptome-based manner.

Bulk RNA seq from Tumor/Spleen. Fourteen days after CT-2A tumor implantation, 10 mice were pooled for each n reported in this study, followed by Gr1 magnetic bead isolation, and RNA was isolated using TRIzol (Thermo Fisher Scientific)-based RNA purification. Briefly, 0.2 ml of chloroform was added to TRIzol samples; top RNA-containing layer was precipitated with 70% isopropanol. Pellets were dried, then resuspended in sterile water, and sent for analysis. Isolated samples were sent to Novogene, which analyzed RNA for quality and provided all data as total counts and fpkm (fragments per kilobase per million reads) values.

Ex vivo TAMgeneration and quantitative RT-PCR. TAMs were generated as described previously (71). Briefly, bone marrow precursors were isolated from the femurs of C57BL/6 mice and resuspended in RPMI supplemented with recombinant granulocyte-macrophage colony-stimulating factor (GM-CSF) (40 ng/ml) and 50% of 0.2 μm sterile-filtered CT-2A supernatant. After 3 days, cells were washed and replaced with the same medium. At 6 days, cells were validated to be phenotypically similar to TAMs in vivo.

LIPU/MB. The LIPU/MB sonication procedure for mouse experiments was performed using a preclinical LIPU device (SonoCloud Technology) manufactured by CarThera and was previously deNAibed (39). Mice were anesthetized with ketamine/xylazine cocktail intraperitonially (ketamine 100 mg/kg, xylazine 10 mg/kg). Microbubbles (MB, Lumason, Bracco) were reconstituted according to manufacturer instructions and injected at a dose of 7.5 mL/kg through the retro-orbital route. Shortly after MB administration, mice were quickly (<10 seconds) placed supine upon the ultrasound transducer holder and the sonication began. A 1 MHz, 10-mm diameter flat ultrasound transducer was fixed in a holder filled with degassed water and sonications were performed transcranially. Sonications were performed for 120 seconds using a 25,000-cycle burst at a 1 Hz pulse repetition frequency and an acoustic pressure of 0.3 MPa as measured in water. After sonications, mice were moved to a clean cage, placed upon a heating pad, and monitored until they recovered from anesthesia.

Statistical Analyses Statistical analyses were performed using Prism Software 9.4.1 (GraphPad). Unpaired Student's t-test was used to compare statistical differences between two groups. For Kaplan-Meier survival curves, log-rank (Mantel-Cox) test was adapted to determine significances between groups. Statistical significances were presented in P-value or P<0.05 was considered significant, *P<0.05, **P<0.01, ***P<0.001, ****P<0.0001.

### Example 2: DOX Upregulates Antigen-Presenting Molecules in GBM

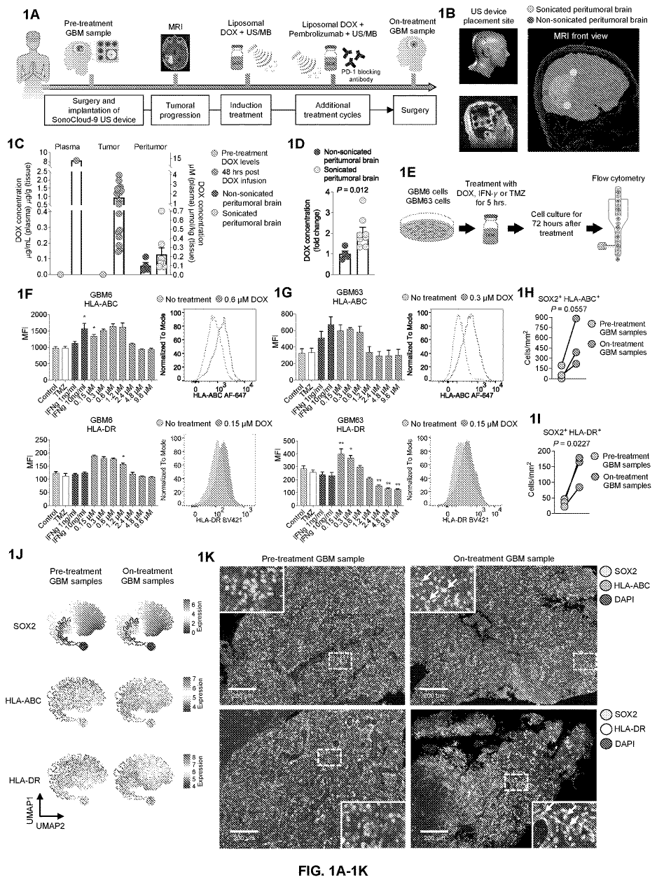

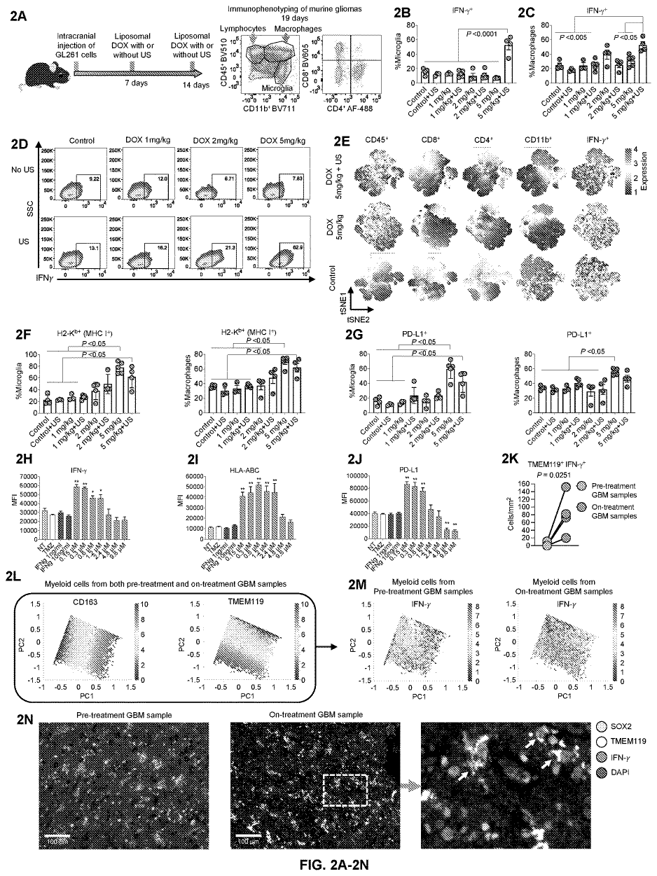

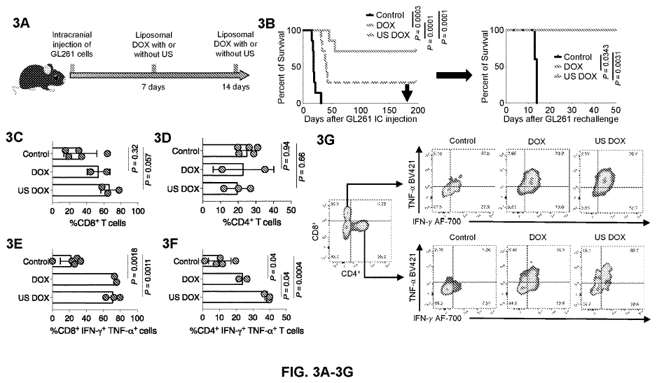

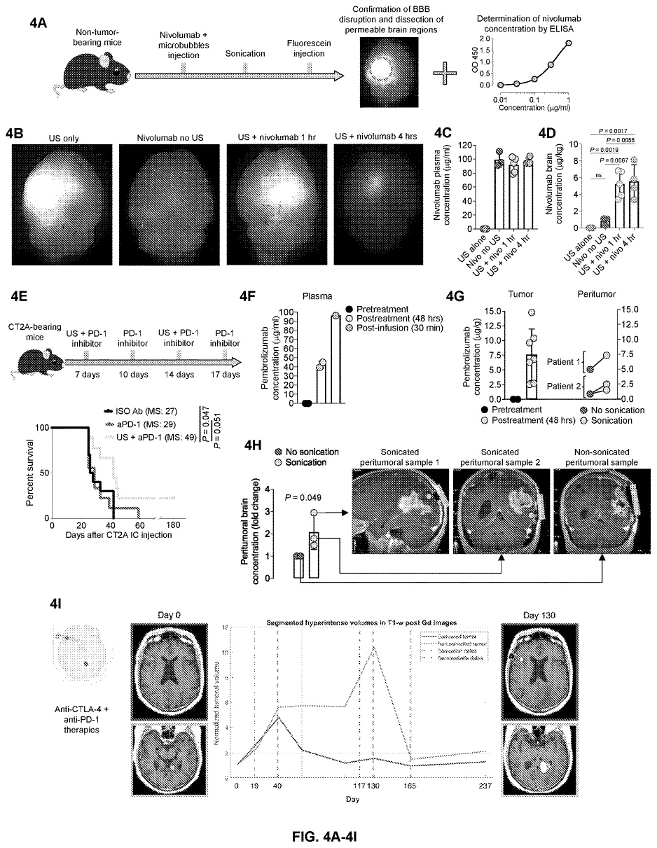

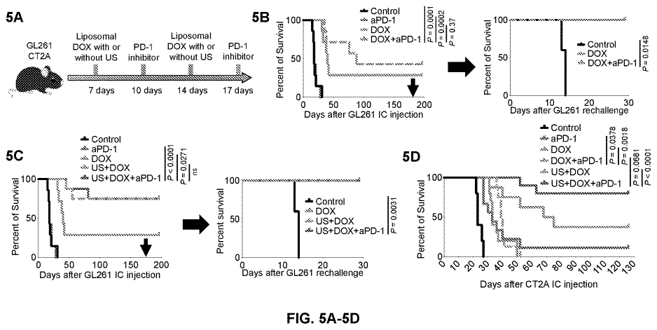

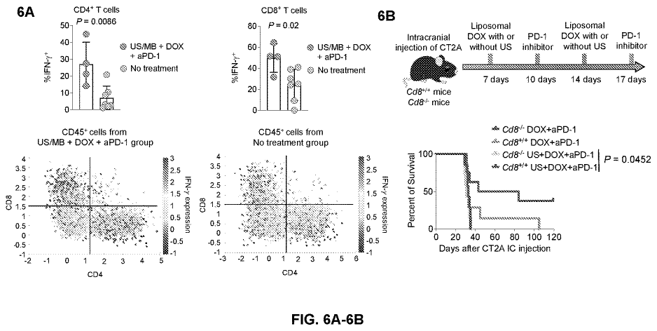

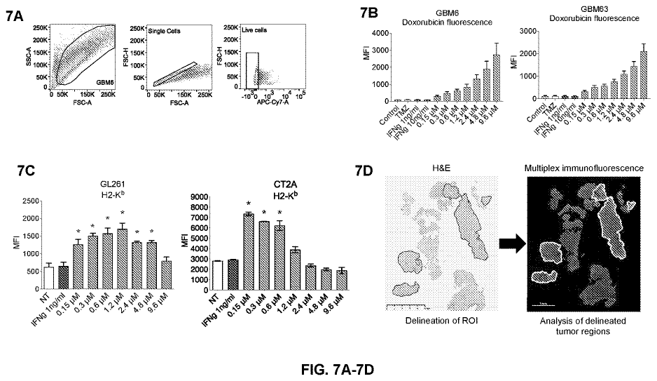

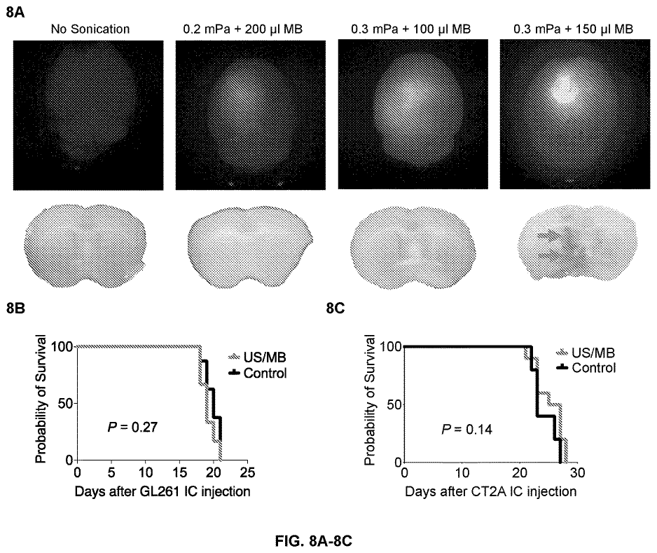

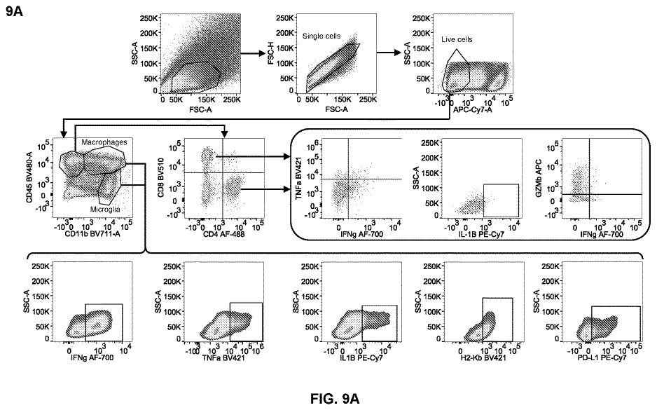

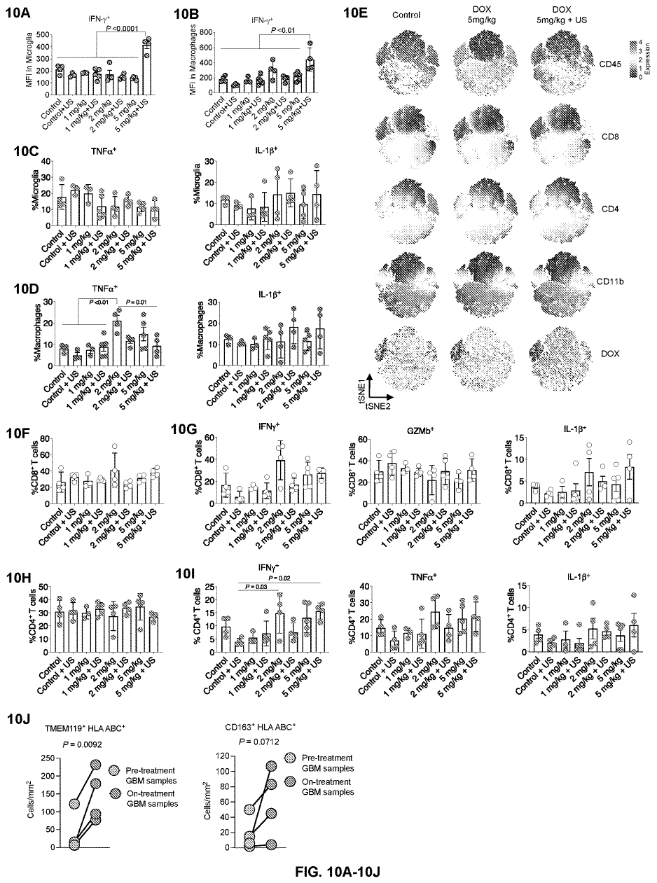

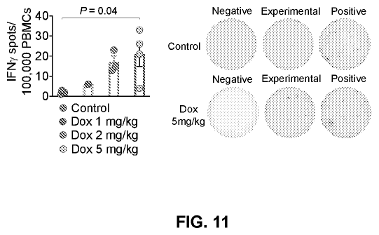

The present example demonstrates the ability of the US/MB technology to increase the concentrations of DOX in the brain of GBM patients and that DOX upregulates antigen-presenting molecules in GBM. Evidence of safety of BBB opening after US has been demonstrated in GBM patients (Idbaih, A., et al., Clin Cancer Res 25, 3793-3801 (2019); Carpentier, A., et al., Sci Transl Med 8, 343re342 (2016)). However, demonstration of increased drug levels in the human brain with the use of US/MB is limited. A novel dose-escalation phase 1 clinical trial has determined the maximum tolerated dose of albumin-bound paclitaxel with the use of an innovative US device called SonoCloud-9 in recurrent Isocitrate Dehydrogenase (IDH) wild-type GBM patients (NCT04528680). In this trial, the US device was implanted in the skull during surgery in a position in which the US waves covered the tumor and surrounding peritumoral brain. During this clinical study, there were four patients that experienced tumoral progression and were candidates for either additional tumoral resection or biopsy. Thus, to leverage the opportunity of the skull implantation of the SonoCloud-9 US device, these patients started salvage therapy with liposomal DOX and PD-1 blockade with pembrolizumab in combination with US/MB to treat the new recurrent tumor derived from clinical evidence suggesting efficacy of anthracyclines in combination with anti-PD-1 therapy (Voorwerk, L., et al., Nat Med 25, 920-928 (2019); Akamatsu, H., et al. JTO Cin Res Rep 2, 100184 (2021)) (FIG. 1A). Overall, these patients underwent induction treatment with 30 mg/m² of liposomal DOX delivered with US/MB followed by subsequent cycles of liposomal DOX plus 200 mg of pembrolizumab delivered with US/MB prior to surgeries. These therapies were well tolerated with no serious adverse events during each cycle. In this context, drug concentration analysis and immunological comparisons between the tumor samples acquired when these patients were enrolled in the phase I clinical trial before treatment with DOX (pre-treatment GBM sample) and the tumor samples resected during treatment with liposomal DOX and pembrolizumab (on-treatment GBM sample) was performed (FIG. 1A). In addition, to determine whether US/MB increases DOX concentrations in the brain of GBM patients. In two of these patients, we were able to obtain peritumoral brain regions covered by the US waves (sonicated) and peritumoral brain regions that were outside the sonication field (non-sonicated) determined by the position of the US device in the skull (FIG. 1B). Of note, sonicated peritumoral brain samples, non-sonicated peritumoral brain samples, and on-treatment GBM samples were acquired 48 hours after treatment with liposomal DOX and pembrolizumab delivered with US/MB. DOX plasma levels detected were of 8.23 μg/mL (14.18 μM) and tumor concentrations ranged from 0.146 μg/g (0.251 μmol/kg) to 2.288 μg/g (3.944 μmol/kg) (FIG. 1C). Whereas there was great variability in the tumor concentration of DOX, it was determined that sonicated peritumoral brain samples had a 2.033-fold increase (95% CI of mean: 1.406-2.659) in DOX concentration relative to non-sonicated peritumoral brain samples (P=0.012, chi-square; FIG. 1D). Overall, these results show the feasibility and ability of the US/MB technology to increase the concentrations of DOX in the brain of GBM patients.

An important factor to maximize the therapeutic effects from enhanced delivery of DOX by US/MB is the association of increased tumor immunogenicity with specific drug concentrations achieved in the brain. One of the immune related effects of DOX relies on increasing the expression of MHC class I on tumor cells (Alagkiozidis, I., et al., J Transl Med 7, 104 (2009); Takayama, T., et al., (Doxil. Pharmaceutics 12(2020)). This particular feature of DOX is relevant as defects in the antigen processing and presentation machinery as well as HLA I expression are frequently found in gliomas (Arrieta, V. A., et al., Oncoimmunology 7, e1445458 (2018)). The effects of a wide range of DOX concentrations in inducing the expression of the antigen-presenting molecules in GBM was evaluated. The PDX cell lines, GBM6 and GBM63, were treated with the following range of DOX concentrations: 9.6, 4.8, 2.4 1.2, 0.6, 0.3, 0.15 and 0 M. Treatments with human IFNγ at concentrations of 1 and 10 ng/ml were included as positive controls. GBM6 and GBM63 cells were exposed to DOX for 5 hours, incubated for additional 72 hours in drug-free media followed by flow cytometry analysis (FIG. 1E and FIG. 7A, FIG. 7B). The DOX concentration range of 0.15-1.2 M upregulated HLA ABC and HLA DR expression relative to the no treatment group in GBM6 cells (adjusted P<0.05, one-way ANOVA; FIG. 1F). In GBM63, it was determined that the DOX concentration range of 0.15-0.6 M had the same effect in upregulating the expression of HLA ABC and HLA-DR (adjusted P<0.05, one-way ANOVA; FIG. 1G). Notably, these DOX immunogenic concentration ranges overlapped with the concentrations attained in the sonicated peritumoral brain regions (FIG. 1C). In contrast, temozolomide (TMZ) did not upregulate these antigen-presenting molecules in PDX cell lines underscoring a specific immunogenic effect of DOX mechanism of action (FIG. 1F and FIG. 1G). In the murine glioma models GL261 and CT2A, a similar DOX concentration range upregulated H-2K^{b}, the murine ortholog of HLA I, compared to the no treatment group (P<0.05, one-way ANOVA; FIG. 7C).

Whether human GBM samples treated with liposomal DOX in combination with US/MB had the same effects in increasing antigen-presenting molecules by glioma cells was investigated. Multiplex immunofluorescence was utilized to evaluate the abundance of SOX2⁺ (tumor cell marker, Yuan, J., et al., Genome Med 10, 57 (2018)) cells expressing HLA ABC and HLA-DR. This analysis was performed in tumor regions delineated by a neuropathologist (FIG. 7D). An increase in cell density of SOX2⁺ HLA ABC⁺ cells (P=0.0557, t test; FIG. 1H) and SOX2⁺ HLA-DR⁺ cells (P=0.0227, t test; FIG. 11) in GBM samples treated with liposomal DOX compared to pre-treatment GBM samples was found (FIGS. 1H and 1I). Notably, although the upregulation of HLA ABC and HLA-DR was not homogenous among all tumor cells, this phenomenon occurred in a considerable proportion of the glioma cell population (FIG. 1J).

Representative images illustrating the expression of HLA ABC and HLA-DR by SOX2⁺ cells are presented in FIG. 1K. Overall, these results show that DOX in combination with US/MB has the ability to increase the expression of antigen presenting molecules in tumor cells from human GBM samples. Furthermore, in pharmacodynamic terms, the upregulation of HLA ABC and HLA DR by cultured GBM cells occurred at a specific DOX concentration range that can be achieved in peritumoral brain regions subjected to sonication with US/MB in GBM patients.

### Example 3: DOX Plus US/MB Modulates Glioma-Associated Microglia and MDMs

The present example demonstrates a robust immunological activity of DOX in brain tumors that is only present when the US/microbubble (MB) technology is implemented. US/MB has proven to be a safe technology for BBB disruption in GBM patients (Idbaih, A., et al., Clin Cancer Res 25, 3793-3801 (2019); Carpentier, A., et al., Sci Transl Med 8, 343re342 (2016)). Nevertheless, to establish safer sonication parameters for improved drug delivery in C57BL/6 mice, a toxicity study evaluating MB dosing and sonication pressure (mPa) was performed. 0.3 mPa with IV administration of 100 L of MB induced a consistent and substantial BBB opening without important harmful effects in the brain (FIG. 8A). Next, whether US/MB without any concomitant treatment had an impact in survival of glioma-bearing mice was evaluated. US/MB alone did not have any effect in survival in the murine glioma models, GL261 (P=0.27, log-rank test; FIG. 8B) and CT2A (P=0.14, log-rank test; FIG. 8C) consistent with previous evidence showing no effect of solely US/MB on survival and modulation of immune cells in mouse glioma models (Sabbagh, A., et al., Clin Cancer Res (2021)).

Similar to the upregulation of antigen presenting molecules, an important factor for successful combination with immunotherapies is the determination of the dose of DOX delivered with US/MB that is able to diffuse into brain to modulate tumor-infiltrating immune cells in gliomas. Therefore, to uncover the DOX concentrations achieved with US/MB needed to induce a proinflammatory tumor microenvironment and considering the concentration-dependent effects in upregulating antigen-presenting molecules in GBM cells, different doses of liposomal DOX (1, 2, and 5 mg/kg) were evaluated with and without US/MB using the GL261 murine glioma model. Following treatment, microglia, macrophages, and T cells residing in the brain were analyzed using flow cytometry (FIG. 2A and FIG. 9A). S5 mg/kg of liposomal DOX led to increased production of IFNγ by microglia (gated on CD45− CD11b+) (P<0.0001, one-way ANOVA, FIG. 2B and FIG. 10A), as well as in monocyte-derived macrophages (P<0.05, one-way ANOVA, FIG. 2C and FIG. 10B). Interestingly, no other doses of DOX nor the 5 mg/kg of dose delivered in the absence of US/MB elicited IFN-γ by glioma-associated myeloid cells. Representative flow cytometry plots of the increase in IFN-γ by myeloid cells in different doses of DOX with and without US/MB are shown in FIG. 2D. Additionally, whole visualization of live cells in tSNE plots revealed that, regardless of whether they were CD45⁺ or CD45−, CD11b+ myeloid cells were the predominant source of IFNγ relative to other cells in the group of 5 mg/kg of liposomal DOX+US/MB (FIG. 2E). The production of TNFα and IL-10 by these glioma-associated myeloid cells was also evaluated. However, no remarkable differences were observed between groups in microglia (FIG. 10C) and monocyte-derived macrophages (FIG. 10D).

Whether DOX fluorescence could be detected in brain cells from these mice was analyzed by flow cytometry. Interestingly, a higher intensity of DOX fluorescence was observed in cells treated with liposomal DOX with US/MB relative to the treatment without US/MB as well as the no treatment group, suggesting greater DOX penetration in the brain when using US/MB (FIG. 10E). The immunomodulatory effect of different doses of liposomal DOX in the lymphocyte compartment was also evaluated (FIG. 9A). However, no significant differences were observed in the percentages of CD8+ (FIG. 10F) and those expressing IFN-γ, GZMb, and IL-10 (FIG. 10G). Similarly, the percentages of CD4+ T cells (FIG. 10H) and those expressing IFN-γ, TNFα, and IL-10 (FIG. 101) were similar between groups. These results highlight the value of US/MB to deliver liposomal DOX to reconfigure the phenotype of the most abundant immune population in gliomas.

Considering the robust IFN-γ phenotype exhibited by microglia and monocyte-derived macrophages induced by liposomal DOX plus US/MB, the expression of H-2K^{b}(MHC I) and PD-L1 was investigated, as these proteins are upregulated in response to IFN-γ. H-2K^{b} (MHC I) was upregulated by microglia and monocyte-derived macrophages in the mouse groups that were treated with 5 mg/kg of liposomal DOX regardless of whether the US/MB was used to deliver the chemotherapy (P<0.05, one-way ANOVA, FIG. 2F). Likewise, PD-L1 was upregulated in the groups treated with 5 mg/mL of liposomal DOX with and without US/MB compared to the control groups and those treated with lower doses of the anthracycline (P<0.05, one-way ANOVA, FIG. 2G). Only the group treated with 5 mg/mL of liposomal DOX without US/MB showed an increase in PD-L1 expression by monocyte-derived macrophages (P<0.05, one-way ANOVA, FIG. 2G). To validate these results, the human microglia cell line, HMC3, was utilized to evaluate the production of IFNγ and expression of HLA ABC, and PD-L1 using a wide range of DOX concentrations. Similar to GBM cells, HMC3 cells were treated with a 5-hour pulse of DOX, TMZ, and IFN-γ followed by drug removal, cell culture for 72 hours, and flow cytometry analysis (FIG. 2E). By gating on single live cells, it was determined that at specific DOX concentrations, this anthracycline promoted the production of IFNγ (P<0.05, one-way ANOVA; FIG. 211) and expression of HLA ABC (P<0.0001, one-way ANOVA; FIG. 21) and PD-L1 (P<0.0001, one-way ANOVA; FIG. 2J) compared to the cells that did not receive any treatment.

Next, whether these preclinical observations were reproducible in GBM patients treated with liposomal DOX delivered with US/MB was investigated. Pre-treatment and on-treatment GBM samples were analyzed in which the expression of IFN-γ and HLA I by TMEM119 (microglial marker) and CD163 (myeloid cell marker) cells using multiplex immunofluorescence was evaluated. It was determined that TMEM119⁺ IFN-γ⁺ cells were more abundant in on-treatment GBM samples compared to pre-treatment GBM samples (P=0.0251, paired t test; FIG. 2K). We bioinformatically isolated myeloid cells (TMEM119⁺ and CD163⁺ cells) from pre-treatment and on-treatment GBM samples and projected them in a Principal Component Analysis (PCA) plot (FIG. 2L). It was determined that the production of IFN-γ was more prevalent and pronounced in myeloid cells in on-treatment GBM samples compared to those myeloid cells from pre-treatment GBM samples (FIG. 2M). FIG. 2N shows representative multiplex immunofluorescence images illustrating tumor regions containing SOX2⁺, TMEM119⁺, and IFN-γ⁺ cells in pre-treatment and on-treatment GBM samples. With regards to HLA I, it was determined that an increased number of TMEM119⁺ (P=0.0092, paired t test) and CD163⁺ cells (P=0.0712, paired t test) expressing HLA I in tumors exposed to DOX relative to tumors resected before DOX treatment (FIG. 10J).

In sum, these results show a novel finding of IFN-γ production by glioma-associated myeloid cells induced by treatment with liposomal DOX only when this drug is delivered with US/MB. Notably, the production of IFN-γ was observed in a microglial cell line as well as in myeloid cells from glioma-bearing mice and GBM patients treated with this anthracycline. In addition, there was an incremental effect in the expression of MHC I in human and mouse glioma associated microglia and macrophages. This evidence uncover the robust immunological activity of DOX in brain tumors that is only present when the US/MB technology is implemented.

### Example 4: Liposomal DOX Plus US/MB Leads to Long-Term Survivors in Glioma-Bearing Mice

The present example demonstrates the immunostimulatory properties of DOX in both the myeloid and lymphocyte compartment, in particular brain tumors, such as gliomas

Whether US/MB can enhance the efficacy of liposomal DOX in preclinical glioma models was evaluated. GL261 glioma cells were intracranially injected in C57BL/6 mice and treated with liposomal DOX delivered with and without US/MB (FIG. 3A). Considering the IFN-γ phenotype by glioma-associated microglia and macrophages achieved with 5 mg/kg of liposomal DOX, the same dose was utilized for survival studies. Two treatments of liposomal DOX delivered with US/MB led to long-term remission of 75% of mice bearing intracranial gliomas compared to about 28.5% of mice treated with liposomal DOX without US/MB (P=0.001, log-rank test, FIG. 3B). To further characterize the immune response and evaluate whether DOX leads to long-lasting immune surveillance, GL261 cells were injected in the contralateral hemisphere of the brain of DOX-treated long-term survivor mice as well as age-matched controls. Only mice previously treated with liposomal DOX regardless of US/MB survived upon intracranial tumor rechallenge (P=0.0343 for DOX vs control, P=0.0031 for DOX+US/MB vs control, FIG. 3B). These results underscore the immune memory exhibited in the brain derived by DOX immune activity that is enhanced with the use of US/MB.

Considering the association between immune memory and adaptive immunity as well as the influence of DOX on lymphocytes (Mattarollo, S. R. et al., Cancer Res 71, 4809-4820 (2011)), immunophenotyping of T cells from long-term survivors was completed. Thus, to provide an additional stimulation for immune response in the brain, glioma cells were injected into long-term survivors and age-matched controls. An increase in the percentage of CD8⁺ T cells in long-term survivors treated with liposomal DOX and US/MB compared to control mice, though the P value was outside of statistical significance (P=0.057, one-way ANOVA; FIG. 3C) and no significant changes in the percentage of CD4⁺ T cells among groups (P=0.94 for DOX vs control, P=0.66 for US/MB+DOX vs control, one-way ANOVA; FIG. 3D). However, CD8⁺ T cells from the long-term survivor mice treated with liposomal DOX with and without US/MB exhibited an IFN-γ⁺ TNF-α⁺ phenotype (P=0.0018 for DOX vs control, P=0.0011 for US/MB+DOX vs control, one-way ANOVA, FIG. 3E). Interestingly, there was a higher percentage of CD4 IFN-γ⁺ TNF-α⁺ T cells from mice treated with DOX alone compared to the control group (P=0.04, one-way ANOVA, FIG. 3F), the greatest percentage of T cells characteristic of a Th1 phenotype was exhibited by long-term survivors treated with liposomal DOX delivered by US/MB (P=0.04 for US/MB+DOX vs DOX, P=0.0004 for US/MB+DOX vs control, one-way ANOVA; FIG. 3F). FIG. 3G shows representative flow cytometry plots of CD4⁺ and CD8 IFN-γ⁺ TNF-α cells in different treatment groups.

Lastly, whether a tumor-specific response by peripheral blood mononuclear cells (PBMCs) could be elicited with liposomal DOX treatment was evaluated. GL261-bearing mice were treated with different doses of liposomal DOX followed by isolation of PBMCs. PBMCs isolated from blood from tumor-bearing mice were then exposed to GL261 tumor lysate. By employing ELISpot assay, it was determined that PBMCs exposed to the highest dose of liposomal DOX (5 mg/kg) were the only ones that showed increased secretion of IFNγ (P<0.04, one-way ANOVA; FIG. 11), showing a stronger activation of PBMCs against tumoral antigen in the context of exposure to the highest dose of DOX.

These results emphasize the immunological power of DOX to promote survival in glioma-bearing mice. In addition to the local effects of DOX in the brain achieved by US/MB in modulating microglia and monocyte-derived macrophages, this drug also exerts its immunological functions peripherally leading to IFN-γ⁺ production by PBMCs. Of note, an increased percentage of CD4IFN-γ⁺ TNF-α⁺ T cells was found in the brains of long-term survivors that were treated with DOX delivered with US/MB showing the potential of this combinatorial therapy. Hence, the immunomodulatory effects and efficacy of DOX in both the myeloid and T cells populating gliomas relies on the effective introduction of this drug into the brain by US/MB.

### Example 5: US/MB Increase the Brain Concentration and Efficacy of PD-1 Antibody

The present example demonstrates preclinical and clinical results showing the ability of US/MB to enhance the penetration of therapeutic antibodies into the brain to generate intracranial responses and potential efficacious effect in extending survival.

Whether US/MB can enhance the penetration of immune checkpoint antibodies into the brain to potentiate their therapeutic efficacy was evaluated. Non-tumor bearing C57BL/6 mice were treated with the human IgG4 antibody that binds the PD-1 receptor, nivolumab, followed by US/MB. Fluorescein was injected soon after sonication for visualization of areas of BBB disruption that were further dissected and analyzed with a nivolumab-specific ELISA (FIG. 4A). As a control, a group that received nivolumab and fluorescein, but did not receive sonication, and another group that received US/MB followed by fluorescein but without nivolumab administration were utilized. In view of evidence showing delayed detection of monoclonal antibodies in bran metastases treated with focused US/MB (Meng, Y. et al., Sci Transl Med 13, eabj4011 (2021)), two time points (1 and 4 hours) were included for determination of nivolumab concentration after sonication. The concentration of nivolumab was measured in BBB disrupted regions in the groups that were subjected to sonication. One brain hemisphere was analyzed for the control group that received nivolumab without US/MB (FIG. 4B). Whereas nivolumab concentration in the plasma were similar (FIG. 4C), the groups that received nivolumab and US/MB had increased concentrations of the human aPD-1 in sonicated areas compared to the group that was treated with nivolumab, but did not receive US/MB (1 hour: 6.3-fold increase, 4 hours: 6.6-fold increase relative to the Nivolumab without US/MB group, P<0.01, one-way ANOVA; FIG. 4D). Of note, no differences in nivolumab concentration was found between murine brains that were extracted 1 and 4 hours after sonication, suggesting that the penetration of this immunotherapy occurs within the first hour of BBB opening.

The molecular weight of nivolumab (150 kDa) is very similar to the murine anti-PD-1 (146 kDa). The antitumoral effects of enhanced delivery of aPD-1 by US/MB in a preclinical glioma model were investigated. Intracranial injection of CT2A glioma cells in C57BL/6 mice was performed. The mice were then treated with IV injection of murine aPD-1 followed by US/MB. A modest increase was observed in survival that included a subset of long-term survivors of mice treated with anti-PD-1 plus US/MB compared to mice treated with the isotype IgG2a antibody (P=0.047, log-rank test) and the anti-PD-1 antibody treatment group (P=0.051, long-rank test) (FIG. 4E).

To determine the added value of US/MB in increasing anti-PD-1 antibody concentrations in the brain of GBM patients, pembrolizumab levels in plasma, tumor, and peritumoral brain were measured in two GBM patients that underwent sonication with the US device, SonoCloud-9, concomitantly with pembrolizumab administration. By employing a pembrolizumab-specific ELISA, it was determined that the anti-PD-1 antibody concentration in the serum of these GBM patients after 48 hours of treatment was 39.450 and 45.099 μg/mL (FIG. 4F), which was similar and in the range determined by previous clinical studies (Portnow, J., et al., JAMA Oncol (2020); Patnaik, A., et al.; Clin Cancer Res 21, 4286-4293 (2015)). The concentrations of pembrolizumab delivered with US/MB in different areas of the resected tumor were evaluated. As a negative and technical controls, pembrolizumab concentration in pre-treatment tumor samples from the same GBM patients. The mean pembrolizumab concentration was 47.588 μg/g (95% CI of mean: 3.53-11.64 μg/g) in these tumors (FIG. 4G). To further assess the ability of US/MB in increasing concentration of anti-PD-1 antibody in the brain, pembrolizumab concentrations were measured in peritumoral regions that were subjected to sonication and in those that were not subjected to sonication. In the two patients that we were able to acquire peritumoral brain samples during surgery, there was an increase of pembrolizumab concentration in the sonicated peritumoral brain regions compared to the non-sonicated ones (FIG. 4G). Specifically, there was a 2.076-fold increase in pembrolizumab concentration in peritumoral brain regions that were covered by the sonication field of the SonoCloud-9 US device relative to those taken outside the sonication field (P=0.049, chi-squared; FIG. 4H). With these assays, an estimate of the pembrolizumab tumoral concentrations is provided that can be achieved after sonication with the SonoCloud-9 US device in GBM patients. These results also show the feasibility and the ability of US/MB to increase the concentration of anti-PD-1 antibody in peritumoral brain regions of GBM patients.

As part of an ongoing clinical trial to treat patients with brain metastases from melanoma with a combination of US-mediated BBB disruption and immune checkpoint blockade (NCT04021420), a 64-year-old patient with two brain tumor masses located in the frontal and occipital regions was included for skull implantation of a SonoCloud-1 US device (CarThera, Paris, France). The zone targeted by the implanted SonoCloud-1 US emitter was modeled with a cylindrical region of interest (ROI) of a 10 mm diameter and 55 mm length covering the frontal lobe tumor mass and part of its margin as shown in FIG. 4I. Conversely, the occipital lobe tumor mass was not contemplated for sonication, The patient received three infusions every three weeks of ipilimumab (3 mg/kg) and nivolumab (1 mg/kg) prior to sonication. The patient received three sonications in combination with intravenous injections of MB (0.1 mL/kg) to disrupt the 13B areas covered by the ROI on days 19, 40, and 117 after implantation of the SonoCloud-1 US device. The tumor mass targeted by the SonoCloud-1 began to shrink after the second sonication, while the non-sonicated tumor mass continued to grow throughout all three sonications and only began to shrink after the patient received GammaKnife treatment on day 130. Thereafter, the patient remained stable without treatment for 10 months after the last ipilimumab and nivolumab infusion.

In sum, the integration of these preclinical and clinical results shows the ability of US/MB to enhance the penetration of therapeutic antibodies into the brain to generate intracranial responses and extend survival. Furthermore, this methodology provides a reliable approach to determine the concentration of nivolumab and pembrolizumab in human brain tumors after enhanced delivery by US/MB in clinical trials.

### Example 6: US/MB Delivery of DOX Potentiates Response to Anti-PD-1 Antibody in Glioma-Bearing Mice

One proposed limitation for resistance to anti-PD-1 antibody in GBM is the immunosuppressive nature of myeloid cells that infiltrate these tumors. The specific IFNγ response of microglia and monocyte-derived macrophages after treatment with liposomal DOX in combination with US/MB shows that these immune cells can be modulated to render a proinflammatory tumor milieu. In addition, the described preclinical efficacy derived from enhanced delivery of anti-PD-1 antibody by US/MB suggests that the combination with liposomal DOX could intensify immune responses in the brain. Therefore, to investigate whether US/MB can enhance the response to DOX and anti-PD-1 antibody, GL261 glioma cells were injected intracranially in C57BL/6 mice. The mice were then treated with liposomal DOX in combination with anti-PD-1 antibody delivered with and without US/MB (FIG. 5A). Initially, the therapeutic effect of combining anti-PD-1 antibody with liposomal DOX without US/M1B was investigated in glioma-bearing mice. Although no effect was observed in survival with aPD-1, the combination of liposomal DOX and anti-PD-1 antibody extended survival compared to the control group (P=0.0001, log-rank test; FIG. 5B). Long-term survivors treated with liposomal DOX with and without anti-PD-1 remained alive after rechallenged with intracranial injection of GL261 cells compared to age-matched controls that succumbed due to tumor growth.

It was then evaluated whether US/MB increase the therapeutic efficacy of liposomal DOX and anti-PD-1 antibody in gliomas. Liposomal DOX delivered with US/MB resulted in cure of 75% of mice compared to the control group (P<0.0001, log-rank test) and the group treated with liposomal DOX without US/MB (P=0.0271, log-rank test; FIG. 5C). The addition of anti-PD-1 antibody to DOX plus US/MB showed similar results in generating the same percentage of long-term survivors suggesting that there was limited room for additional efficacy when including the immune checkpoint antibody (FIG. 5C). Thus, the proposed combination was evaluated in a different glioma model. CT2A-bearing mice were treated with the same therapeutic scheme used previously for GL261 (FIG. 5A). In this model, it was found that the combination of liposomal DOX+anti-PD-1 antibody without US/MB extended survival compared to the liposomal DOX group (P=0.0018, log-rank test) and the anti-PD-1 antibody group (P=0.0378, log-rank test; FIG. 5D). The addition of anti-PD-1 antibody to DOX plus US/MB led to remarkable therapeutic enhancement denoted as long-term survival percentage of 80% relative to mice treated with DOX plus US/MB without immunotherapy (P<0.0001, log-rank test; FIG. 5D). It was also observed that the antitumoral efficacy of liposomal DOX and anti-PD-1 antibody was further potentiated with the use of US/MB compared to the group treated with liposomal DOX+anti-PD-1 antibody without US/MB (P=0.0681, log-rank test; FIG. 5D).

In sum, these results show the immunological synergy that DOX and anti-PD-1 antibody can display in gliomas when they are effectively introduced the brain. Furthermore, these preclinical experiments employing two glioma models highlight the therapeutic advantage of employing US/MB to deliver chemotherapies and immunotherapies into the brain to increase survival rates.

### Example 7: Generation of an IFN-γ⁺ Profile in Human Glioma-Infiltrating CD8+ and CD4+ T Cells

The present example demonstrates generation of an IFN-7⁺ profile in human glioma-infiltrating CD8+ and CD4+ T cells. In addition, considering the abundance of CD4⁺ IFN-γ⁺ TNF-α⁺ T cells in long-term survivors treated with liposomal DOX delivered with US/MB (FIG. 3F), the phenotype of tumor-infiltrating T cells in GBM patients treated and not treated with this combinatorial therapy in combination with US/MB was investigated. Flow cytometry analysis of CD45⁺ T cells that were further gated on CD8⁺ and CD4⁺ T cells was performed (FIG. 12). It was determined that CD8⁺ and CD4⁺ T cells from GBMs treated with liposomal DOX and pembrolizumab delivered with US/MB exhibited a higher expression and percentage of IFN-γ (P=0.0086 for CD4⁺ T cells and P=0.02 for CD8⁺ T cells, unpaired t test; FIG. 6A) compared to T cells derived from GBMs that did not receive any treatment before surgery.

Then, to determine the relevance of CD8⁺ T cells for therapeutic efficacy in the context of DOX plus anti-PD-1 antibody delivered with US/MB, a survival experiment was performed using CT2A glioma cells to test the proposed anthracycline and immune checkpoint blockade in the context of US/MB in Cd8^{-/-} and Cd8^{+/+} C57BL/6 mice. A survival benefit was only observed in mice that had intact immunity treated with the combinatorial therapy delivered with US/MB, whereas none of the mice absent of CD8⁺ T cells survived despite treatment (P=0.0452, log-rank test; FIG. 6B). This shows that efficacy of DOX and anti-PD-1 antibody relies on CD8⁺ T cells, and that this therapeutic effect can be enhanced with the use of US/MB. Furthermore, evidence is shown that liposomal DOX and pembrolizumab delivered with the SonoCloud-9 US device induce an IFN-γ⁺ phenotype on T cells from GBM patients.

### Example 8: FcγR Binding Characteristics of an Fc-Enhanced Anti-Mouse CTLA4 Antibody Surrogate

The discordance observed with aCTLA-4 therapy between mouse models and humans may be attributed to variations in FcγR-dependent mechanisms and IgG subclass affinity for FcγR subtypes, which differ between these species (29). To account for this, Applicant compared the FcγR binding characteristics of a human IgG1 FcE-aCTLA-4 (hIgG1.DLE) and an IgG2b mouse aCTLA-4 antibody engineered with the same mutations in the Fc-region as botensilimab, to an unmodified human IgG1 and mouse IgG2b variant respectively (FIG. 13, Table 1). The binding to activating (A) and inhibitory (I) IgG Fc receptors by surface plasmon resonance (SPR) was used to establish the A/I ratio (Table 2 and Table 3), a measure shown to be predictive of cytotoxicity in vivo (43).

Here, Applicant observed that the mouse FcE-aCTLA-4 bound with higher affinity to FcγRIV (FIG. 13D), the mouse ortholog of human FcγRIIIA, with an average equilibrium dissociation constant (K_{D}) of 1.7 nM compared to 61.3 nM for the unmodified mouse IgG2b aCTLA-4 antibody (FIG. 13C). Binding affinity to the inhibitory mouse FcγRIIB was found to be similar between FcE-aCTLA-4 (K_{D}=7.11 M; FIG. 13B) and the corresponding unmodified aCTLA-4 (K_{D}=5.88 M; FIG. 13A). The improved binding affinity by mouse FcE-aCTLA-4 to mouse FcγR as measured by SPR was further confirmed by flow cytometry for binding to Chinese Hamster Ovary (CHO) cells ectopically expressing mouse FcγRI, FcγRIIB, FcγRIII and FcγRIV. Compared to the unmodified mouse IgG2b aCTLA-4 antibody, the FcE-aCTLA-4 demonstrated superior potency and maximal cell binding to cell-expressed mouse FcγRI (FIG. 13E), FcγRIIB (FIG. 13F), FcγRIII (FIG. 13G) and FcγRIV (FIG. 13H).

Similarly, the human FcE-aCTLA-4 antibody bound with significantly higher affinity than the parental human IgG1 variant to human FcγRIIIA and showed a ˜2-fold increase in binding affinity to human FcγRIIB (Table 2; FIG. 18). Notably, the human FcE-aCTLA-4 antibody showed significantly improved binding to low affinity FcγRIIIA polymorphic variant (F158) with an average K_{D} of 2.83 nM compared to 91.9 nM for the corresponding unmodified human IgG1 variant (Table 2). Binding to the high affinity FcγRIIIA polymorphic variant (V158) was also improved with the human FcE-aCTLA-4 antibody (K_{D}=2.76 nM) compared to the unmodified human IgG1 aCTLA-4 antibody (K_{D}=27.6 nM) (Table 2; FIG. 18). By comparing the A/I ratios (Table 2 and Table 3), Applicant's data suggest that the mouse IgG2b aCTLA-4 antibody would exhibit in vivo cytotoxicity comparable to that of clinically relevant human IgG1 aCTLA-4 antibodies. In contrast, the mouse FcE-aCTLA-4 containing the same modifications in the Fc region as botensilimab, would have enhanced FcγR-mediated effector functions and potentially improved therapeutic efficacy.

### Example 9: FcγRs and CTLA-4 Expression in Murine and Human Glioma Tumor Microenvironment

To characterize the expression of FcγRIIIA and CTLA-4 in GBM, Applicant used a publicly available single-cell RNA-sequencing (scRNA-seq) dataset of human GBM (44) (FIG. 14A). First, unsupervised clustering was performed to identify various cell types including lymphocytes (T cells, Tregs, B cells, and NK cells), myeloid cells (macrophages, monocytes, microglia), dendritic cells, granulocytes, endothelial cells, oligodendrocytes, and tumor cells (FIG. 14A). Applicant then evaluated the expression of FcγRIIIA and CTLA-4 in these populations and observed that FcγRIIIA was mostly expressed by myeloid cells, including granulocytes, macrophages, microglia, and monocytes. In contrast, CTLA-4 was preferentially expressed by T cells and Tregs (FIGS. 14B and C). To verify these findings at the protein level, Applicant used multiplex immunofluorescence on human GBM specimens. Applicant confirmed that most FcγRIIIA-positive cells co-expressed myeloid lineage markers including CD11c, CD68, or CD163 (FIGS. 14D and E). By scRNA-seq analysis, Applicant further confirmed the expression of these markers (FIG. 19).

Applicant further investigated the expression of different FcγRs in the CT-2A murine glioma model using scRNA-seq. Following a similar approach as with human GBM samples, Applicant performed unsupervised clustering to characterize various cell types isolated from mouse brains (FIG. 14F). Consistent with the expression pattern in humans, Fcgr4 (FcγRIV), the mouse ortholog of human FCGR3A, was preferentially expressed in tumor-associated macrophages/microglia (TAM) (FIG. 14G), whereas CTLA-4 was found to be highly expressed by T cells, particularly by Tregs (FIGS. 14H and I). Lastly, Applicant compared the expression of Fcgrl, Fcgr2, Fcgr3, and Fcgr4 in tumor-infiltrating and systemic Gr1+ myeloid cells isolated from CT-2A glioma tumor-bearing (45). Notably, Applicant observed that FcγRs were upregulated in tumor-infiltrating myeloid cells compared to splenic myeloid cells (FIG. 14J). Similarly, Applicant observed significantly higher Fcgr4 gene expression, as measured by quantitative reverse transcription-polymerase chain reaction (qRT-PCR), in TAMs generated ex vivo by conditioning bone marrow-derived macrophages (BMDM) with CT-2A supernatant, compared to unconditioned BMDM (FIG. 20).

### Example 10: Induction of Fc-Enhanced Anti-CTLA-4 Mediated Treg Depletion in Glioma Microenvironment

Having confirmed the enriched expression of CTLA-4 in intra-tumoral Tregs and FcγRIV in TAMs, Applicant assessed whether the enhanced affinity of FcE-aCTLA-4 to FcγRIV could be harnessed to deplete Tregs within the glioma tumor microenvironment.

First, an in vitro phagocytosis assay was performed with TAM generated exposing BMDM to CT-2A-conditioned media. These TAMs were then co-cultured with GFP-expressing splenic CD4⁺Foxp3⁺ Tregs or CD4⁺Foxp3⁻ non-Tregs in the presence of FcE-aCTLA-4, the unmodified parental aCTLA-4, or IC IgG antibodies (FIG. 15A). In this assay, FcE-aCTLA-4 promoted phagocytosis of CD4⁺Foxp3⁺ Tregs compared to the IC IgG antibody, which was not observed with parental aCTLA-4 (FIG. 15B). Depletion was specific to CD4⁺Foxp3⁺Tregs, as Applicant did not observed any phagocytosis of the CD4⁺Foxp3⁻ non-Tregs (FIG. 15C), consistent with the lower cell surface expression of CTLA-4 on non-Treg cells (46).

To confirm the ability of FcE-aCTLA-4 to selectively deplete intra-tumoral Tregs in vivo, CT-2A tumor-bearing mice were treated with FcE-aCTLA-4, parental aCTLA-4, or IC IgG antibodies twice a week for two weeks and immunophenotyped the brain-resident and systemic lymphocytes on days 14 and 21 post-tumor implantation with flow cytometry (FIG. 15D and FIG. 21). Consistent with Applicant's observations in vitro, FcE-aCTLA-4 promoted superior depletion of intra-tumoral Tregs compared to parental aCTLA-4 or IC IgG, an effect that was more pronounced at day 21 compared to day 14 post tumor implantation (FIGS. 15E and 15F). In contrast, splenic Tregs were not affected by treatment, consistent with the lower peripheral expression of CTLA-4 and Fcgr4 compared to that of the TME (FIG. 14J). Notably, the FcE-aCTLA-4-mediated depletion of intra-tumoral Tregs was accompanied by a significant decrease in PD-1 expression by tumor-infiltrating CD4⁺ (FIG. 15G) and CD8⁺ T cells (FIG. 1511), whereas PD-1 expression was unaltered in splenic lymphocytes (FIG. 22).

### Example 11: Superior Anti-Tumor Efficacy of Fc-Enhanced Anti-CTLA-4 Antibody in Murine Glioma Models

To evaluate whether the enhanced ability of FcE-aCTLA-4 to engage activating FcγRIV on TAMs and promote depletion of tumor-infiltrating Tregs correlated with efficacy, survival studies were performed in three distinct immune-competent murine GBM models GL261, CT-2A, and QPP4. Whereas CT2-A and QPP4 have been established as immunotherapy-resistant models, variable immune-resistant as well as immune-susceptible phenotype has been described for GL261 gliomas.(47-49) Mice were treated with FcE-aCTLA-4, parental aCTLA-4, or IC antibodies twice a week for two weeks (FIG. 16A). In GL261 tumor-bearing mice, aCTLA-4 antibodies slightly improved the median survival from 18 days post implantation in the IC-treated mice to 20 and 21 days with aCTLA-4 (p=0.0135) and FcE-aCTLA-4 (p=0.0090), respectively (FIG. 16B). In QPP4 and CT-2A tumor-bearing mice, FcE-aCTLA-4 therapy promoted curative responses in the majority of treated mice with long-term survival in 70% (p=0.0002; FIG. 16C) and 80% (p<0.0001; FIG. 4D), within each model, respectively. In contrast, treatment with the parental aCTLA-4 showed no significant improvement in overall survival compared to control in QPP4 tumor-bearing mice (FIG. 16C) and only 2 of 10 complete responses in CT-2A tumor-bearing mice (FIG. 16D).

CTLA-4 blockade has been shown to enhance CD8⁺ T cell memory formation, function, and long-term maintenance in viral (50) and tumor-challenged mouse models (51). To assess the ability of aCTLA-4 therapy to trigger immune memory response in glioma and prevent recurrence, tumor-bearing mice cured of CT-2A tumors were re-challenged 120 days after initial tumor implantation with a new contralateral intracranial glioma implant and assessed for survival in the absence of any further treatment. All FcE-aCTLA-4 treated mice survived subsequent tumor re-challenge, suggesting that FcE-aCTLA-4 treatment stimulated a long-lasting immune memory response that was capable of rejecting the second tumor implant (FIG. 16D, right). To further characterize this long-lasting anti-tumor response, the brains of re-challenged long-term survivors were isolated and the brain-resident immune cells were profiled by flow cytometry. Compared to non-treated controls, the brains of long-term survivors were characterized by a lower myeloid-to-lymphocyte ratio (p<0.0273; FIG. 4E, left) and a higher ratio of CD8⁺ to CD4⁺ infiltrating T cells (p<0.0001; FIG. 16E, right). In addition, CD8⁺ T cells isolated from treated mice were in an activated state characterized by a high production of interferon gamma (IFN-γ) and a low expression of PD-1, compared to CD8⁺ T cells isolated from non-treated mice (p=0.0434, FIG. 16F, right). This enhanced CD8⁺ T cell response in FcE-aCTLA-4 treated mice was further confirmed by immunohistochemistry. Here, Applicant observed that the increased in tumor infiltrating CD8⁺ T cells mediated by FcE-aCTLA-4 therapy were localized to the gliotic or lesional region of the brain (FIG. 16G). Notably, and consistent with the improved effector function of FcE-aCTLA-4, Applicant observed a decrease in tumor-infiltrating Foxp3⁺ Tregs in mice treated with FcE-aCTLA-4 (FIG. 1611). Together, these experiments demonstrate that the enhanced FcγR-mediated effector functions recruited by an FcE-aCTLA-4 antibody promotes superior therapeutic efficacy compared to conventional aCTLA-4 therapy. This improved efficacy correlated with the ability to remodel the TME towards an active cytotoxic state characterized by reduced myeloid and Treg compartments and increased CD8 T cell activity.

### Example 12: Enhanced FcγRIIIA Expression with Doxorubicin Delivered Using Ultrasound-Mediated BBB Opening

DOX has been shown to enhance the response to PD-1 blockade in breast cancer (38). To investigate whether DOX could further improve the efficacy of FcE-aCTLA-4, Applicant investigated the effect of DOX and LIPU/MB on the expression of FcγRIIIA in human GBM. This was done in four recurrent GBM patients that received DOX+aPD-1 with concomitant LIPU/MB. These patients, who were initially enrolled in Applicant's NCT04528680 clinical trial, had a SonoCloud-9 (SC9) ultrasound device implanted in the skull at the time of resection of recurrent GBM followed by multiple cycles of albumin-bound paclitaxel delivered with LIPU/MB every three weeks until recurrence (42). The SC9 is a skull-implantable ultrasound used for LIPU/MB that has been shown to open the BBB in humans for enhancing drug delivery to the brain by approximately 4-6 times (42). The patients Applicant evaluate here, had progressed on the prior clinical trial with LIPU/MB, and therefore had SC9 already implanted at time of tumor progression. Upon recurrence following this trial-related treatment, as part of a single-patient expanded access protocol, these patients received induction with liposomal DOX (30 mg) and LIPU/MB, and within 10-14 days, underwent a second dose of DOX (30 mg) and aPD-1 (pembrolizumab 200 mg), with concomitant LIPU/MB BBB opening. All 4 patients underwent DOX/aPD-1 plus LIPU/MB for 1-3 cycles every 3 weeks, followed by surgery for tumor resection or biopsy. This allowed for the availability of tissue prior to treatment with DOX and aPD-1 (pre-DOX GBM samples) obtained at time of SC9 implant (during surgery for clinical trial), and tumor tissue resected after 1-3 cycles of DOX and aPD-1 (during-DOX GBM samples). In all cases, surgery was performed 2 days after DOX administration with LIPU/MB.

Through multiplex immunofluorescence, Applicant observed that DOX+aPD-1 treatment with concomitant LIPU/MB led to an increase in the frequency of FcγRIIIA⁺ CD68⁺ (p<0.0138) and CD11c⁺ (p<0.0071) TAMs, compared to the tissue obtained before this treatment in all 4 patients (FIGS. 17A, B and C).

The tumor concentration of DOX was also measured two days after administration of DOX plus LIPU/MB and the concentration reached up to 4 mol/kg (0.25-3.95 mol/kg). It is plausible that substantially higher concentrations were achieved directly after administration (FIG. 17D).

To verify that the upregulation of FcγRIIIA resulted from DOX exposure, Applicant subsequently examined the effect of DOX exposure on the expression of FCGR3A in the human microglia cell line HMC3, which Applicant also found to express myeloid cell/macrophage marker CD68 (FIG. 23). An exposure to DOX for 5 hours followed by incubation in fresh media for 72 hours led to a concentration-dependent increase in FCGR3A expression, with levels up to nearly 16 times higher than in untreated microglia (FIG. 17E, F). These findings were further validated at the protein level by flow cytometry. The presence of DOX in these cells (determined by DOX auto-fluorescence) was observed for at least 72 hours following drug exposure (FIG. 17G, left). The expression of FcγRIIIA was upregulated by DOX exposure at concentrations as low as 0.1 M (FIG. 17G, right).

To investigate the safety of the suggested combination therapy with LIPU/MB, FcE-aCTLA-4, DOX, and aPD-1, a toxicity study with healthy mice was performed in which the treatment was administered twice weekly for two weeks (total of 4 administrations; FIG. 24). Body weight and survival were monitored throughout the treatment period and for a total of 50 days. All mice tolerated treatment and made it to the endpoint on day 50 when mice were euthanized, and the brains were collected for histological evaluation of central nervous system (CNS) toxicity. Multiple courses of combination treatment with LIPU/MB at the concentrations studied were well-tolerated and did not result in body weight changes or signs of CNS pathology (FIG. 24). No difference in toxicity was observed between mice receiving the first-generation parental aCTLA-4 and FcE-aCTLA-4, respectively (FIG. 24).

Applicant then investigated the efficacy of this combination in GL261-bearing mice, as this model was less susceptible to monotherapy with FcE-aCTLA-4 compared to the QPP4 and CT-2A models (FIGS. 16B, C, D). The combination of FcE-aCTLA-4 with aPD-1 improved the survival of GL261-bearing mice compared to monotherapy with FcE-aCTLA-4 alone and resulted in 14.3% long-term survivors (p=0.0021). Survival was further improved when FcE-aCTLA-4 and aPD-1 were combined with DOX, which resulted in 85.7% long-term survivors (p=0.0002; FIG. 17H). Applicant also found that when combined with LIPU/MB, treatment with FcE-aCTLA-4 and aPD-1 doubled the proportion of long-term survivors (33.3% vs 14.3%) compared to treatment without LIPU/MB (FIGS. 17I and J). Similarly, treatment with LIPU/MB+IC+DOX resulted in 16.7% long-term survivors (FIG. 17J). The strongest survival benefit was observed following LIPU/MB-enhanced delivery of FcE-aCTLA-4, aPD-1, and DOX, which resulted in 90.9% of long-term survivors (FIG. 17J). Applicant observed similar findings with the QPP4 mouse model (FIG. 25).

### Example 13

In this study, Applicant identified a novel and highly effective immunotherapy strategy that proved to be efficacious for multiple murine glioma models, most of which are poorly responsive or resistant to conventional immune checkpoint blockade therapy. Central to this approach, is an FcE-aCTLA-4 antibody that is designed to leverage novel FcγR-dependent mechanisms of action to promote optimal T cell priming and activation, T cell memory, and selectively reduce the frequency of intra-tumoral Tregs superior to that seen with conventional aCTLA-4 antibodies (Ref. Chand et al., personal communication) (24, 52, 53). Previous studies have also shown curative responses in ICB-resistant mouse models of microsatellite stable (MSS) colon cancer, melanoma, breast, and pancreatic tumors (24, 53). A human analog, botensilimab, is currently under clinical investigation in several trials for advanced solid tumors (NCT05529316, NCT04121676, NCT05630183, NCT05608044, NCT05377528, NCT03860272, NCT04028063, NCT05672316, NCT05627635) and has shown unprecedented clinical activity across nine different ‘cold’ and ICB refractory cancers, and a favorable safety and tolerability profile (33, 54).

Doxorubicin, an immune-modulatory chemotherapeutic, upregulated FcγRIIIA on tumor-infiltrating TAMs in glioblastoma patients treated with doxorubicin, anti-PD-1 and low-intensity ultrasound and microbubbles (LIPU/MB), a procedure that temporarily opens the blood-brain barrier. Treatment of immunotherapy-resistant murine gliomas with a combination of FcE-aCTLA-4, anti-PD-1, and doxorubicin with concomitant LIPU/MB resulted in over 90% cure rates, which correlated with increased infiltration of activated CD8+ T cells, and complete rejection upon subsequent tumor rechallenge. Applicant's findings indicate that FcE-aCTLA-4 exhibits a novel immunomodulatory function in glioblastoma, and its therapeutic potential is significantly improved when combined with anti-PD-1, doxorubicin, and LIPU/MB. This novel strategy offers a potential breakthrough in glioma treatment, warranting further evaluation in clinical trials.

Botensilimab is designed to enhance binding to human FcγRs, in particular, FcγRIIIA which has been shown to be critical for the activity of aCTLA-4 antibodies (24, 29). Here Applicant used a mouse analog of botensilimab, FcE-aCTLA-4, engineered with the same DLE mutations in the Fc region to enhance binding to FcγRIV, the mouse ortholog of human FcγRIIIA. Antibody co-engagement of FcγRs can either activate or inhibit immune responses; and compared to its unmodified mouse IgG2b aCTLA-4 variant, the FcE-aCTLA-4 had a significantly higher A/I ratio, a measure previously shown to be predicative of cytotoxicity in vivo (43). Applicant demonstrate that the FcE-aCTLA-4 promoted superior anti-tumor activity than a conventional aCTLA-4 antibody, in three distinct immunotherapy-resistant murine orthotopic glioma models: GL261, CT-2A and QPP4. Considering that the FcγRIIIA V158F single-nucleotide polymorphism has been linked to better clinical outcomes with ipilimumab in inflamed tumors (29), Applicant's data suggest that an FcE-aCTLA-4 antibody may offer a wider therapeutic range and effectiveness, regardless of FcγRIIIA allele status. This is due to the enhanced binding to both variants, as evidenced by Phase 1 clinical trial reports for botensilimab (Chand et al., personal communication) (53, 54).

The enhanced anti-tumor activity that Applicant observed by FcE-aCTLA-4 antibody, was accompanied by a reduction of intra-tumoral Tregs, decreased myeloid-to-lymphocyte ratio, and increased CD8-to-CD4 ratio. Consistent with the ability of ICB to reinvigorate dysfunctional TILs and augment their anti-tumor effects (55, 56), Applicant's data also show that FcE-aCTLA-4 therapy enhanced the activation and cytotoxic capacity of the tumor infiltrating CD8⁺ T cells which were characterized by elevated IFN-γ expression and reduced PD-1 expression. As reported in other murine tumor-bearing models and clinical studies, high PD-1 expression on CD8⁺ T cells is a hallmark of dysfunction, whereas PD-1 low T cells exhibit greater effector function and tumor killing capacity (55, 57, 58). Moreover, and consistent with the ability of aCTLA-4 therapy to promote durable responses in cancer (59), FcE-aCTLA-4 treatment in the mouse glioma models led to long-term survival and formation of immune memory that protected against tumor recurrence and rechallenge without the need for further treatment.

In ‘cold’ and poorly immunogenic tumors, including gliomas, TAMs and intra-tumoral Tregs are major barriers to T cell infiltration and anti-tumor function. These immune cell populations play a major role in promoting tumor progression and have been associated with resistance to ICB therapy (8, 60-62). Applicant's study demonstrates that TAMs in both human and mouse gliomas express FcγRIIIA and FcγRIV, respectively whereas CTLA-4 expression is notably higher in intra-tumoral Tregs. Interestingly, the suppressive effects of Tregs and TAMs in glioma may not be mutually exclusive, as studies in GBM suggest that CTLA-4-mediated immune suppression could be related to the infiltration of macrophages within the tumor microenvironment (63). In addition to decreasing the myeloid-to-lymphocyte ratio in mouse models of glioma, Applicant's data suggests that the FcE-aCTLA-4, via its increased binding to mouse FcγRIV, is optimized to mediate potent cross-linking of CTLA-4-expressing intra-tumoral Tregs and FcγRIV-expressing phagocytic TAMs to deplete intra-tumoral Tregs. This activity was not seen with the unmodified aCTLA-4 antibody and may explain why conventional aCTLA-4 antibodies, ipilimumab or tremelimumab, have shown limited clinical activity in patients with GBM (8, 63).

However, monotherapy responses and long-term survival following FcE-aCTLA-4 treatment differed among glioma models. Notably, all GL261 tumor-bearing mice treated with FcE-aCTLA-4 monotherapy succumbed to their disease, suggesting that combination therapy may be needed to overcome tumor-immune heterogeneity. To address this, Applicant evaluated the efficacy of FcE-aCTLA-4 in combination with aPD-1, DOX, a chemotherapeutic agent shown to potentiate the response to aPD-1 in patients with breast cancer (38) and LIPU/MB, a procedure that temporarily opens the blood-brain barrier and improves drug delivery to the brain (39, 40, 42) in GL261 tumor-bearing mice. While the combination of FcE-aCTLA-4 and aPD-1 modestly improved long-term survival, combining FcE-aCTLA-4 with aPD-1, DOX, and LIPU/MB resulted in durable anti-tumor response in approximately 90% of treated-mice. Applicant hypothesized that this significantly improved response could be attributed not only to increase drug concentration mediated by LIPU/MB, but also by the potential that DOX upregulates the expression of activating FcγRs on TAMs and promotes immunogenic cell death, stimulating an immune response that synergizes with ICB therapy (64). To support this, Applicant presents evidence that in patients with recurrent GBM, the systemic administration of 30 mg of DOX with LIPU/MB leads to an upregulation of FcγRIIIA expression in TAMs. Additionally, exposure to DOX at concentrations observed within human tumors leads to increased FcγRIIIA expression in a human microglia cell line. Notably, Applicant's data suggest that DOX could be leveraged to enhance the response to aCTLA-4 by upregulating FcγRs which are critical for its activity (24, 29, 65). This observation suggest that administration of DOX might lead to improvement in efficacy of other antibody-related forms of immunotherapy where the Fc region binding FcγR in immune cells is important for their mechanism of action.

There are some limitations to Applicant's study. i) Glioma cell lines and mouse models, which may not fully recapitulate the biology of the tumor-immune microenvironment of human GBM. For instance, orthotopic mouse tumor models are less infiltrative than human tumors, as such the BBB may not be as completely intact as it is human tumors following gross total resection. Consequently, there may be an underestimation of the role of LIPU/MB in murine models compared to its role in humans, where Applicant previously demonstrated a 4 to 6-fold increase in parenchymal chemotherapy concentrations (42). Furthermore, mouse models tend to be more immunogenic than human GBM, which may account for the discrepancy between the success of immunotherapies in preclinical studies and their subsequent failures in clinical trials. ii) While Applicant demonstrated that the therapeutic activity of FcE-aCTLA-4 in glioma correlated with enhanced depletion of intra-tumoral Tregs (66, 67), several other mechanisms independent of Tregs, such as T cell (24) and myeloid activation (30) may be important contributors to efficacy. In fact, enhancing the interaction between FcγRs and the Fc region of aCTLA-4 antibodies was shown to be crucial for inducing a robust and durable response by antigen-specific T cells. This was achieved by improving the quality of the immune synapse between a T cell and an FcγRIIIA-expressing APC, as evidenced by enhanced intensity of duration of T cell receptor signaling (24, 68).

In conclusion, Applicant's study highlights a novel therapeutic approach for the treatment of glioma using an FcE-aCTLA-4 that promotes superior immune-activation and durable anti-tumor responses than conventional ICB. This effect was associated with the depletion of intra-tumoral Tregs by TAMs and enhanced CD8⁺ T cell anti-tumor responses. Moreover, Applicant demonstrated that the expression of FcγRIIIA in TAMs, a receptor that was previously established to be critical for the activity of aCTLA-4 therapy (24), can be increased by DOX. Notably, the therapeutic efficacy of FcE-aCTLA-4 is significantly enhanced when combined with DOX, aPD-1, and LIPU/MB, while maintaining tolerability. Collectively, these results and the promising efficacy of botensilimab (FcE-aCTLA-4) observed in on-going clinical trials in other solid tumors, provide a compelling rationale for investigating this novel immunotherapy strategy with LIPU/MB in upcoming clinical trials for GBM patients.

TABLE 1
Summary of Fc isotype, Fc mutations and Fcγ receptor
(FcγR) binding characteristics to human FcγRIIIA
or mouse FcγRIV of anti-human and anti-mouse CTLA4 antibodies.

| | Anti-CTLA-4 | | | FcγR Binding |
|---|---|---|---|---|
| | Antibody | Fc Isotype | Fc Mutations | Characteristics |
| Human | Parental | IgG1 | — | Low |
| | Botensilimab | IgG1.DLE | S239D.A330L.I332E | >FcγRIIIA binding |
| | (Fc-enhanced) | | | |
| Murine | 9D9 Parental | mIgG2b | — | Low |
| Surrogates | 9D9 FcE | mIgG2b.DLE | S241D.A332L.I334E | >FcγRIV binding |
| | (Fc-enhanced) | | | |

TABLE 2
Binding affinities of anti-human CTLA-4 antibodies to human FcγRIIB,
FcγRIIIA F158 and FcγRIIIA V158 proteins determined by SPR.
The ratio of binding of an IgG subtype to activating FcγRs and
inhibitory FcγRIIB known as the activating to inhibitory (A/I) ratio are shown.

| | Human | | | | |
|---|---|---|---|---|---|
| | FcγRIIB | FcγRIIIA F158 | FcγRIIIA V158 | IIIA F158/IIB | IIIA V158/IIB |
| | Antibody | | | | |
| | KD (M) | KD (M) | KD (M) | A/I | A/I |
| hIgG1 | 6.86E−06 | 9.19E−08 | 2.76E−08 | 75 | 249 |
| hIgG1-DLE | 3.23E−06 | 2.83E−09 | 2.76E−09 | 1141 | 1170 |

TABLE 3
Binding affinities of anti-mouse CTLA-4 antibodies to mouse FcγRII
and FcγRIV proteins determined by SPR. The ratio of binding
of an IgG subtype to activating FcγRs and inhibitory FcγRIIB
known as the activating to inhibitory (A/I) ratio are shown.

| | Mouse | | |
|---|---|---|---|
| | FcγRIIB | FcγRIV | IV/IIB |
| | Antibody | | |
| | KD (M) | KD (M) | A/I |
| mIgG2b | 5.80E−06 | 6.13E−08 | 95 |
| mIgG2b-DLE | 7.11E−06 | 1.70E−09 | 4182 |

### REFERENCES

1. Stupp R, Taillibert S, Kanner A, Read W, Steinberg D, Lhermitte B, et al. Effect of Tumor-Treating Fields Plus Maintenance Temozolomide vs Maintenance Temozolomide Alone on Survival in Patients With Glioblastoma: A Randomized Clinical Trial. JAMA. 2017; 318(23):2306-16.
2. Miller K D, Ostrom Q T, Kruchko C, Patil N, Tihan T, Cioffi G, et al. Brain and other central nervous system tumor statistics, 2021. CA Cancer J Clin. 2021; 71(5):381-406.
3. Janjua T I, Rewatkar P, Ahmed-Cox A, Saeed I, Mansfeld F M, Kulshreshtha R, et al. Frontiers in the treatment of glioblastoma: Past, present and emerging. Adv Drug Deliver Rev. 2021; 171:108-38.
4. Reardon D A, Brandes A A, Omuro A, Mulholland P, Lim M, Wick A, et al. Effect of Nivolumab vs Bevacizumab in Patients With Recurrent Glioblastoma: The CheckMate 143 Phase 3 Randomized Clinical Trial. JAMA Oncol. 2020; 6(7):1003-10.
5. Lim M, Xia Y, Bettegowda C, and Weller M. Current state of immunotherapy for glioblastoma. Nat Rev Clin Oncol. 2018; 15(7):422-42.
6. Bagley S J, Kothari S, Rahman R, Lee E Q, Dunn G P, Galanis E, et al. Glioblastoma Clinical Trials: Current Landscape and Opportunities for Improvement. Clin Cancer Res. 2022; 28(4):594-602.
7. Khasraw M, Reardon D A, Weller M, and Sampson J H. PD-1 Inhibitors: Do they have a Future in the Treatment of Glioblastoma?Clin Cancer Res. 2020; 26(20):5287-96.
8. Arrieta V A, Dmello C, McGrail D J, Brat D J, Lee-Chang C, Heimberger A B, et al. Immune checkpoint blockade in glioblastoma: from tumor heterogeneity to personalized treatment. J Clin Invest. 2023; 133(2).
9. Carpentier A, Canney M, Vignot A, Reina V, Beccaria K, Horodyckid C, et al. Clinical trial of blood-brain barrier disruption by pulsed ultrasound. Sci Transl Med. 2016; 8(343):343re2.
10. Pitz M W, Desai A, Grossman S A, and Blakeley J O. Tissue concentration of systemically administered antineoplastic agents in human brain tumors. J Neurooncol. 2011; 104(3):629-38.
11. Aldape K, Brindle K M, Chesler L, Chopra R, Gajjar A, Gilbert M R, et al. Challenges to curing primary brain tumours. Nat Rev Clin Oncol. 2019; 16(8):509-20.
12. DiDomenico J, Lamano J B, Oyon D, Li Y, Veliceasa D, Kaur G, et al. The immune checkpoint protein PD-L1 induces and maintains regulatory T cells in glioblastoma. Oncoimmunology. 2018; 7(7):e1448329.
13. Larkin C J, Arrieta V A, Najem H, Li G, Zhang P, Miska J, et al. Myeloid Cell Classification and Therapeutic Opportunities Within the Glioblastoma Tumor Microenvironment in the Single Cell-Omics Era. Front Immunol. 2022; 13:907605.
14. Mirzaei R, Sarkar S, and Yong V W. T Cell Exhaustion in Glioblastoma: Intricacies of Immune Checkpoints. Trends Immunol. 2017; 38(2):104-15.
15. Habashy K J, Mansour R, Moussalem C, Sawaya R, and Massaad M J. Challenges in glioblastoma immunotherapy: mechanisms of resistance and therapeutic approaches to overcome them. Br J Cancer. 2022; 127(6):976-87.
16. Wang Q, Hu B, Hu X, Kim H, Squatrito M, Scarpace L, et al. Tumor Evolution of Glioma-Intrinsic Gene Expression Subtypes Associates with Immunological Changes in the Microenvironment. Cancer Cell. 2017; 32(1):42-56 e6.
17. Ravi V M, Will P, Kueckelhaus J, Sun N, Joseph K, Salie H, et al. Spatially resolved multi-omics deciphers bidirectional tumor-host interdependence in glioblastoma. Cancer Cell. 2022; 40(6):639-55 e13.
18. Zhao J, Chen A X, Gartrell R D, Silverman A M, Aparicio L, Chu T, et al. Immune and genomic correlates of response to anti-PD-1 immunotherapy in glioblastoma. Nat Med. 2019; 25(3):462-9.
19. Arrieta V A, Chen A X, Kane J R, Kang S J, Kassab C, Dmello C, et al. ERK1/2 phosphorylation predicts survival following anti-PD-1 immunotherapy in recurrent glioblastoma. Nat Cancer. 2021; 2(12):1372-86.
20. Johanns T M, Miller C A, Dorward I G, Tsien C, Chang E, Perry A, et al. Immunogenomics of Hypermutated Glioblastoma: A Patient with Germline POLE Deficiency Treated with Checkpoint Blockade Immunotherapy. Cancer Discov. 2016; 6(11):1230-6.
21. Ranjan S, Quezado M, Garren N, Boris L, Siegel C, Neto O L A, et al. Clinical decision making in the era of immunotherapy for high grade-glioma: report of four cases. Bmc Cancer. 2018; 18.
22. Omuro A, Vlahovic G, Lim M, Sahebjam S, Baehring J, Cloughesy T, et al. Nivolumab with or without ipilimumab in patients with recurrent glioblastoma: results from exploratory phase I cohorts of CheckMate 143. Neuro-Oncology. 2018; 20(5):674-86.
23. Korman A J, Garrett-Thomson S C, and Lonberg N. The foundations of immune checkpoint blockade and the ipilimumab approval decennial. Nat Rev Drug Discov. 2022; 21(7):509-28.
24. Waight J D, Chand D, Dietrich S, Gombos R, Horn T, Gonzalez A M, et al. Selective FcgammaR Co-engagement on APCs Modulates the Activity of Therapeutic Antibodies Targeting T Cell Antigens. Cancer Cell. 2018; 33(6):1033-47 e5.
25. Sharma A, Subudhi S K, Blando J, Scutti J, Vence L, Wargo J, et al. Anti-CTLA-4 Immunotherapy Does Not Deplete FOXP3(+) Regulatory T Cells (Tregs) in Human Cancers. Clin Cancer Res. 2019; 25(4):1233-8.
26. Chen X, Song X M, Li K, and Zhang T. Fc gamma R-Binding Is an Important Functional Attribute for Immune Checkpoint Antibodies in Cancer Immunotherapy. Front Immunol. 2019; 10.
27. Bruhns P, and Jonsson F. Mouse and human FcR effector functions. Immunol Rev. 2015; 268(1):25-51.
28. Ben Mkaddem S, Benhamou M, and Monteiro R C. Understanding Fc Receptor Involvement in Inflammatory Diseases: From Mechanisms to New Therapeutic Tools. Front Immunol. 2019; 10.
29. Arce Vargas F, Furness A J S, Litchfield K, Joshi K, Rosenthal R, Ghorani E, et al. Fc Effector Function Contributes to the Activity of Human Anti-CTLA-4 Antibodies. Cancer Cell. 2018; 33(4):649-63 e4.
30. Yofe I, Landsberger T, Yalin A, Solomon I, Costoya C, Demane D F, et al. Anti-CTLA-4 antibodies drive myeloid activation and reprogram the tumor microenvironment through FcgammaR engagement and type I interferon signaling. Nat Cancer. 2022; 3(11):1336-50.
31. Bruhns P, Iannascoli B, England P, Mancardi D A, Fernandez N, Jorieux S, et al. Specificity and affinity of human Fcgamma receptors and their polymorphic variants for human IgG subclasses. Blood. 2009; 113(16):3716-25.
32. 33rd Annual Meeting & Pre-Conference Programs of the Society for Immunotherapy of Cancer (SITC 2018) Abstracts. J Immunother Cancer. 2018; 6.
33. El-Khoueiry A, Bullock A, Tsimberidou A, Mahadevan D, Wilky B, Twardowski P, et al. Agen1181, an Fc-Enhanced Anti-Ctla-4 Antibody, Alone and in Combination with Balstilimab (Anti-Pd-1) in Patients with Advanced Solid Tumors: Initial Phase I Results. J Immunother Cancer. 2021; 9:A509-A.
34. Bockorny B, Grossman J E, and Hidalgo M. Facts and Hopes in Immunotherapy of Pancreatic Cancer. Clin Cancer Res. 2022; 28(21):4606-17.
35. Omuro A, Reardon D A, Sampson J H, Baehring J, Sahebjam S, Cloughesy T F, et al. Nivolumab plus radiotherapy with or without temozolomide in newly diagnosed glioblastoma: Results from exploratory phase I cohorts of CheckMate 143. Neurooncol Adv. 2022; 4(1):vdac025.
36. Hau P, Fabel K, Baumgart U, Rummele P, Grauer O, Bock A, et al. Pegylated liposomal doxorubicin-efficacy in patients with recurrent high-grade glioma. Cancer. 2004; 100(6):1199-207.
37. Cohen-Inbar O, Xu Z Y, and Sheehan J P. Focused ultrasound-aided immunomodulation in glioblastoma multiforme: a therapeutic concept. J Ther Ultrasound. 2016; 4.
38. Voorwerk L, Slagter M, Horlings H M, Sikorska K, van de Vijver K K, de Maaker M, et al. Immune induction strategies in metastatic triple-negative breast cancer to enhance the sensitivity to PD-1 blockade: the TONIC trial. Nat Med. 2019; 25(6):920-8.
39. Zhang D Y, Dmello C, Chen L, Arrieta V A, Gonzalez-Buendia E, Kane J R, et al. Ultrasound-mediated Delivery of Paclitaxel for Glioma: A Comparative Study of Distribution, Toxicity, and Efficacy of Albumin-bound Versus Cremophor Formulations. Clin Cancer Res. 2020; 26(2):477-86.
40. Idbaih A, Canney M, Belin L, Desseaux C, Vignot A, Bouchoux G, et al. Safety and Feasibility of Repeated and Transient Blood-Brain Barrier Disruption by Pulsed Ultrasound in Patients with Recurrent Glioblastoma. Clin Cancer Res. 2019; 25(13):3793-801.
41. Sabbagh A, Beccaria K, Ling X Y, Marisetty A, Ott M, Caruso H, et al. Opening of the Blood-Brain Barrier Using Low-Intensity Pulsed Ultrasound Enhances Responses to Immunotherapy in Preclinical Glioma Models. Clin Cancer Res. 2021; 27(15):4325-37.
42. Sonabend A M, Gould A, Amidei C, Ward R, Schmidt K A, Zhang D Y, et al. Repeated blood-bmin barrier opening with an implantable ultrasound device for delivery of albumin-bound paclitaxel in patients with recurrent glioblastoma: a phase 1 trial. Lancet Oncol. 2023; 24(5):509-22.
43. Nimmerjahn F, and Ravetch J V. Divergent immunoglobulin g subclass activity through selective Fc receptor binding. Science. 2005; 310(5753):1510-2.
44. Abdelfattah N, Kumar P, Wang C, Len J S, Flynn W F, Gao R, et al. Single-cell analysis of human glioma and immune cells identifies S100A4 as an immunotherapy target. Nat Commun. 2022; 13(1):767.
45. Miska J, Rashidi A, Lee-Chang C, Gao P, Lopez-Rosas A, Zhang P, et al. Polyamines drive myeloid cell survival by buffering intracellular pH to promote immunosuppression in glioblastoma. Sci Adv. 2021; 7(8).
46. Ha D, Tanaka A, Kibayashi T, Tanemura A, Sugiyama D, Wing J B, et al. Differential control of human Treg and effector T cells in tumor immunity by Fc-engineered anti-CTLA-4 antibody. Proc Natl Acad Sci USA. 2019; 116(2):609-18.
47. Dmello C, Zhao J, Chen L, Gould A, Castro B, Arrieta V A, et al. Checkpoint kinase 1/2 inhibition potentiates anti-tumoral immune response and sensitizes gliomas to immune checkpoint blockade. Nat Commun. 2023; 14(1):1566.
48. Haddad A F, Young J S, Amara D, Berger M S, Raleigh D R, Aghi M K, et al. Mouse models of glioblastoma for the evaluation of novel therapeutic strategies. Neurooncol Adv. 2021; 3(1):vdab100.
49. Chen C H, Chin R L, Hartley G P, Lea S T, Engel B J, Hsieh C E, et al. Novel Murine Glioblastoma Models That Reflect the Immunotherapy Resistance Profile of Human Disease. Neuro Oncol. 2023.
50. Pedicord V A, Montalvo W, Leiner I M, and Allison J P. Single dose of anti-CTLA-4 enhances CD8⁺ T-cell memory formation, function, and maintenance. Proc Natl Acad Sci USA. 2011; 108(1):266-71.
51. Mok S, Duffy C R, and Allison J P. Effects of anti-CTLA-4 and anti-PD-1 on memory T-cell differentiation and resistance to tumor relapse. J Immunol. 2018; 200(1).
52. Tanne A J, Galand C, Abou-Slaybi A, Wilkens M, Marques M, Ng S, et al. Fc-enhanced anti-CTLA-4 antibody, AGEN1181: new mechanistic insights for potent antitumor immunity and combination potential in treatment-resistant solid tumors. Cancer Res. 2021; 81(13).
53. Delepine C, Levey D, Krishnan S, Kim K S, Sonabend A, Wilkens M, et al. Botensilimab, an Fc-Enhanced Ctla-4 Antibody, Enhances Innate and Adaptive Immune Activation to Promote Superior Anti-Tumor Immunity in Cold and I-O Refractory Tumors. J Immunother Cancer. 2022; 10:A490-A.
54. Wilky B, El-Khoueiry A, Bullock A, Tsimberidou A, Mahadevan D, Margolin K, et al. Botensilimab, a Novel Innate/Adaptive Immune Activator, Plus or Minus Balstilimab (Anti-Pd-1) in “Cold” and I-O Refractory Metastatic Solid Tumors. J Immunother Cancer. 2022; 10:A810-A.
55. Wherry E J, and Kurachi M. Molecular and cellular insights into T cell exhaustion. Nat Rev Immunol. 2015; 15(8):486-99.
56. Zarour H M. Reversing T-cell Dysfunction and Exhaustion in Cancer. Clin Cancer Res. 2016; 22(8):1856-64.
57. Kansy B A, Concha-Benavente F, Srivastava R M, Jie H B, Shayan G, Lei Y, et al. PD-1 Status in CD8(+) T Cells Associates with Survival and Anti-PD-1 Therapeutic Outcomes in Head and Neck Cancer. Cancer Res. 2017; 77(22):6353-64.
58. Ma J, Zheng B, Goswami S, Meng L, Zhang D, Cao C, et al. PD1(Hi) CD8(+) T cells correlate with exhausted signature and poor clinical outcome in hepatocellular carcinoma. J Immunother Cancer. 2019; 7(1):331.
59. Harris S J, Brown J, Lopez J, and Yap T A. Immuno-oncology combinations: raising the tail of the survival curve. Cancer Biol Med. 2016; 13(2):171-93.
60. Humphries W, Wei J, Sampson J H, and Heimberger A B. The role of tregs in glioma-mediated immunosuppression: potential target for intervention. Neurosurg Clin NAm. 2010; 21(1):125-37.
61. Kennedy B C, Showers C R, Anderson D E, Anderson L, Canoll P, Bruce J N, et al. Tumor-associated macrophages in glioma: friend or foe?J Oncol. 2013; 2013:486912.
62. Xiang X, Wang J, Lu D, and Xu X. Targeting tumor-associated macrophages to synergize tumor immunotherapy. Signal Transduct Target Ther. 2021; 6(1):75.
63. Guan X, Wang Y, Sun Y, Zhang C, Ma S, Zhang D, et al. CTLA4-Mediated Immunosuppression in Glioblastoma is Associated with the Infiltration of Macrophages in the Tumor Microenvironment. J Inflamm Res. 2021; 14:7315-29.
64. Huang F Y, Lei J, Sun Y, Yan F, Chen B, Zhang L M, et al. Induction of enhanced immunogenic cell death through ultrasound-controlled release of doxorubicin by liposome-microbubble complexes. Oncoimmunology. 2018; 7(7).
65. Waight J D, Chand D, and Savitsky D A. New tricks for old targets: Anti-CTLA-4 antibodies re-envisioned for cancer immunotherapy. Oncotarget. 2018; 9(58):31171-2.
66. Chang A L, Miska J, Wainwright D A, Dey M, Rivetta C V, Yu D, et al. CCL2 Produced by the Glioma Microenvironment Is Essential for the Recruitment of Regulatory T Cells and Myeloid-Derived Suppressor Cells. Cancer Res. 2016; 76(19):5671-82.
67. Miska J, Lee-Chang C, Rashidi A, Muroski M E, Chang A L, Lopez-Rosas A, et al. HIF-1 alpha Is a Metabolic Switch between Glycolytic-Driven Migration and Oxidative Phosphorylation-Driven Immunosuppression of Tregs in Glioblastoma. Cell Rep. 2019; 27(1):226-+.
68. Schneider H, Smith X, Liu H, Bismuth G, and Rudd C E. CTLA-4 disrupts ZAP70 microcluster formation with reduced T cell/APC dwell times and calcium mobilization. Eur J Immunol. 2008; 38(1):40-7.
69. Shingu T, Ho A L, Yuan L, Zhou X, Dai C, Zheng S, et al. Qki deficiency maintains stemness of glioma stem cells in suboptimal environment by downregulating endolysosomal degradation. Nat Genet. 2017; 49(1):75-86.
70. Mancardi D A, Iannascoli B, Hoos S, England P, Daeron M, and Bruhns P. FcgammaRIV is a mouse IgE receptor that resembles macrophage FcepsilonRI in humans and promotes IgE-induced lung inflammation. J Clin Invest. 2008; 118(11):3738-50.
71. Zhang P, Miska J, Lee-Chang C, Rashidi A, Panek W K, An S, et al. Therapeutic targeting of tumor-associated myeloid cells synergizes with radiation therapy for glioblastoma. Proc Natl Acad Sci USA. 2019; 116(47):23714-23.

1.-53. (canceled)

54. A method for treating a glioblastoma in a subject in need thereof comprising:
(a) administering to the subject a therapeutically effective amount of an anthracycline; (b) administering to the subject a therapeutically effective amount of an anti-PD-1 antibody; and
(c) disrupting the blood-brain barrier of the subject by administering low-intensity pulsed ultrasound and microbubbles.

55. The method of claim 54, wherein the therapeutically effective amount of the anthracycline and the low-intensity pulsed ultrasound and microbubbles are administered simultaneously.

56. The method of claim 54, wherein the therapeutically effective amount of the anthracycline, the therapeutically effective amount of the anti-PD-1 antibody, and the low-intensity pulsed ultrasound and microbubbles are administered simultaneously.

57. The method of claim 54, wherein the therapeutically effective amount of anthracycline is administered immediately after the low-intensity pulsed ultrasound and microbubbles are administered.

58. The method of claim 54, wherein the therapeutically effective amount of the anti-PD-1 antibody is administered first, then the low-intensity pulsed ultrasound and microbubbles are administered, and the therapeutically effective amount of the anthracycline is administered last.

59. The method of claim 58, wherein there is a period of time between administering the therapeutically effective amount of the anti-PD-1 antibody and administering the low-intensity pulsed ultrasound and microbubbles.

60. The method of claim 59, wherein the period of time is about 1 hour to about 8 hours.

61. The method of claim 54, wherein the anthracycline is doxorubicin.

62. The method of claim 61, wherein the doxorubicin is liposomal doxorubicin.

63. The method of claim 54, wherein disrupting the blood-brain barrier increases the concentration of the anthracycline in the brain of the subject relative to the concentration of the anthracycline in the brain of the subject in the absence of disrupting the blood-brain barrier.

64. The method of claim 54, wherein disrupting the blood-brain barrier increases the concentration of the anti-PD-1 antibody in the brain of the subject relative to the concentration of the anti-PD-1 antibody in the brain of the subject in the absence of disrupting the blood-brain barrier.

65. The method of claim 54, wherein the microbubbles are administered intravenously.

66. The method of claim 54, wherein the low-intensity pulsed ultrasound is administered by an ultrasound device.

67. The method of claim 66, wherein the ultrasound device is implanted in a cranial window in the skull of the subject.

68. The method of claim 54, wherein the subject was previously treated with a radiotherapy, a chemotherapy, an immunotherapy, or any combination thereof.

69. The method of claim 54, wherein the low-intensity pulsed ultrasound is administered in a plurality of pulsed steps.

70. The method of claim 54, further comprising monitoring the subject for one or more toxicities.

71. The method of claim 54, further comprising assessing disruption of the blood-brain barrier by contrast magnetic resonance imaging.

72. A method for treating a tumor or cancer in a subject in need thereof comprising:
(a) administering to the subject a therapeutically effective amount of an anthracycline; (b) administering to the subject a therapeutically effective amount of checkpoint inhibitor.

73. A method of upregulating the expression of FcγRIIIA in a subject comprising:
(a) administering a therapeutically effective amount of an anthracycline, and (b) administering a therapeutically effective amount of a checkpoint inhibitor to the subject;
wherein the effective amount of the anthracycline is a sub-therapeutic amount.
